SEQUENCE LISTING (36 sequences)

SEQ ID NO: 1 - DNA, 17 bp, Artificial Sequence
  Synthetic polynucleotide
gccgtgaactgggtcga

SEQ ID NO: 2 - DNA, 23 bp, Artificial Sequence
  Synthetic polynucleotide
gcatatatagcaatgtctcctgc

SEQ ID NO: 3 - DNA, 24 bp, Artificial Sequence
  Synthetic polynucleotide
agggttctatggcacatttgatgt

SEQ ID NO: 4 - DNA, 23 bp, Artificial Sequence
  Synthetic polynucleotide
tggcttcttcggtcttcataaac

SEQ ID NO: 5 - DNA, 21 bp, Artificial Sequence
  Synthetic polynucleotide
gctgcggaaacttcaggaaat

SEQ ID NO: 6 - DNA, 23 bp, Artificial Sequence
  Synthetic polynucleotide
agagacgtgtcactcctggactt

SEQ ID NO: 7 - DNA, 20 bp, Artificial Sequence
  Synthetic polynucleotide
acaggaatggccttcatcac

SEQ ID NO: 8 - DNA, 20 bp, Artificial Sequence
  Synthetic polynucleotide
aacatggtgccctgagaaac

SEQ ID NO: 9 - DNA, 19 bp, Artificial Sequence
  Synthetic polynucleotide
ccatcatggatctgcgtgg

SEQ ID NO: 10 - DNA, 21 bp, Artificial Sequence
  Synthetic polynucleotide
cacgagaggaagtctctgcgt

SEQ ID NO: 11 - DNA, 20 bp, Artificial Sequence
  Synthetic polynucleotide
tgacagccaagtccattctg

SEQ ID NO: 12 - DNA, 20 bp, Artificial Sequence
  Synthetic polynucleotide
tatcctccctgaccacttgc

SEQ ID NO: 13 - DNA, 20 bp, Artificial Sequence
  Synthetic polynucleotide
ctgcctgagcaagaatgtga

SEQ ID NO: 14 - DNA, 20 bp, Artificial Sequence
  Synthetic polynucleotide
tgctctgggttttctgcttt

SEQ ID NO: 15 - DNA, 20 bp, Artificial Sequence
  Synthetic polynucleotide
aagggctttcccaaacatga

SEQ ID NO: 16 - DNA, 21 bp, Artificial Sequence
  Synthetic polynucleotide
tgctggtgctcacacttttga

SEQ ID NO: 17 - DNA, 20 bp, Artificial Sequence
  Synthetic polynucleotide
gtggaaagacctctggtgga

SEQ ID NO: 18 - DNA, 20 bp, Artificial Sequence
  Synthetic polynucleotide
tggaaccacctccataggaa

SEQ ID NO: 19 - DNA, 22 bp, Artificial Sequence
  Synthetic polynucleotide
cctagcagctatgaggatccat

SEQ ID NO: 20 - DNA, 17 bp, Artificial Sequence
  Synthetic polynucleotide
cttcggcagcattttcg

SEQ ID NO: 21 - DNA, 21 bp, Artificial Sequence
  Synthetic polynucleotide
tctgtttgcatttctcctggt

SEQ ID NO: 22 - DNA, 20 bp, Artificial Sequence
  Synthetic polynucleotide
ggaactccacaactgccaat

SEQ ID NO: 23 - DNA, 20 bp, Artificial Sequence
  Synthetic polynucleotide
gctaacctctaccgcctcct

SEQ ID NO: 24 - DNA, 20 bp, Artificial Sequence
  Synthetic polynucleotide
ggtcactgtccccatacacc

SEQ ID NO: 25 - DNA, 20 bp, Artificial Sequence
  Synthetic polynucleotide
tgagccccaaggggacgagg

SEQ ID NO: 26 - DNA, 20 bp, Artificial Sequence
  Synthetic polynucleotide
gccgggttcagggtgactgc

SEQ ID NO: 27 - DNA, 21 bp, Artificial Sequence
  Synthetic polynucleotide
tctcctttgagctgtttgcag

SEQ ID NO: 28 - DNA, 18 bp, Artificial Sequence
  Synthetic polynucleotide
caccacatgcttgccatc

SEQ ID NO: 29 - DNA, 21 bp, Artificial Sequence
  Synthetic polynucleotide
gcgacctggaagtccaactac

SEQ ID NO: 30 - DNA, 19 bp, Artificial Sequence
  Synthetic polynucleotide
atctgctgcatctgcttgg

SEQ ID NO: 31 - DNA, 20 bp, Artificial Sequence
  Synthetic polynucleotide
tttccccgatttccttctct

SEQ ID NO: 32 - DNA, 20 bp, Artificial Sequence
  Synthetic polynucleotide
gcgtgtaagacctgggacat

SEQ ID NO: 33 - DNA, 20 bp, Artificial Sequence
  Synthetic polynucleotide
ccccatccagtccttagtca

SEQ ID NO: 34 - DNA, 20 bp, Artificial Sequence
  Synthetic polynucleotide
acttctgggcaggagtcaga

SEQ ID NO: 35 - DNA, 22 bp, Artificial Sequence
  Synthetic polynucleotide
cgagtcaacggatttggtcgta

SEQ ID NO: 36 - DNA, 28 bp, Artificial Sequence
  Synthetic polynucleotide
gcaacaatatccactttaccagagttaa

(19) United States
(12) Patent Application Publication
(10) Pub. No.: US 2017/0020798 A1
(43) Pub. Date: Jan. 26, 2017

(54) HESPERIDIN-CONTAINING COMPOSITIONS AND METHODS FOR TREATMENT OF SKIN DISORDERS

(71) Applicant: The Regents of the University of California, Oakland, CA (US)

(72) Inventors: Peter M. Elias, San Francisco, CA (US); Mao-Qiang Man, San Bruno, CA (US)

(21) Appl. No.: 15/053,833

(22) Filed: Feb. 25, 2016

Related U.S. Application Data
(60) Provisional application No. 62/120,682, filed on Feb. 25, 2015.

(51) Int. Cl. A61K 8/72; A61Q 19/00; A61K 8/63; A61K 8/36; A61K 8/44; A61K 8/34; A61K 8/42; A61Q 19/08; A61K 8/58

(52) U.S. Cl. CPC A61K 8/72; A61Q 19/08; A61Q 19/007; A61K 8/63; A61K 8/585; A61K 8/44; A61K 8/345; A61K 8/42; A61K 8/361; A61K 8/36

(57) ABSTRACT

Compositions and methods for treating aged skin or skin otherwise suffering from abnormal epidermal barrier function are disclosed, where the compositions include hesperidin.

### CROSS-REFERENCES TO RELATED APPLICATIONS

This application claims the benefit of U.S. Provisional Application No. 62/120,682, filed Feb. 25, 2015 which is incorporated herein by reference in its entirety and for all purposes.

### REFERENCE TO A “SEQUENCE LISTING,” A TABLE, OR A COMPUTER PROGRAM LISTING APPENDIX SUBMITTED AS AN ASCII FILE

The Sequence Listing written in file 48536-558001WO_ST25.TXT, created Feb. 18, 2016, 7,435 bytes, machine format IBM-PC, MS-Windows operating system, is hereby incorporated by reference.

### FIELD OF THE DISCLOSED SUBJECT MATTER

The presently disclosed subject matter relates to topical compositions containing hesperidin, cholesterol alone or as part of a triple physiologic mixture of stratum corneum lipids, formulated at an acidic pH, and their utility for the treatment of: i) dry or aged skin due to an abnormal epidermal permeability barrier; ii) dry skin with an abnormal permeability barrier due to the use of systemic hypolipidemic drugs; iii) prevent or treat epidermal functional abnormalities that accompany therapy with immunomodulator treatment; and iii) prevent or treat epidermal functional abnormalities that accompany therapy with glucocorticoid treatment.

### BACKGROUND

One of the main functions of mammalian skin is to serve as a barrier to the systemic uptake of potentially harmful compounds and pathogens. This barrier also serves to conserve water, allowing life in a terrestrial environment. The epidermal permeability barrier resides in the stratum corneum (SC), where hydrophobic lipids are sequestered as multilayered lamellae within the intercellular spaces, which regulate transepidermal water loss, corneocyte cohesion, and percutaneous penetration. Three main lipids form the permeability barrier in the epidermis. These barrier lipids include cholesterol, ceramides and free fatty acids, in an approximately equal molar ratio.

As skin ages, there are negative effects on the dermis (the deeper skin layers) which are evidenced predominantly as cosmetic symptoms, such as wrinkling and sagging. There are also negative effects of aging on the epidermis, particularly on the permeability barrier, where these negative effects favor dermal inflammation and a predilection to infections (as a result of both reduced lipid production and the higher pH of aged skin), resulting in suboptimal permeability and antimicrobial barriers.

As skin ages, the epidermis becomes thinner with reduced epidermal proliferation, abnormal differentiation, impaired lipid synthesis and an elevated skin surface pH. These alterations have profound consequences for barrier function, skin cohesion, antimicrobial defense, inflammatory thresholds, and cutaneous wound healing. These abnormalities have been linked, in part, to reduced epidermal IL-1α expression, reduced epidermal expression of CD44 and its ligand, hyaluronic acid, and reduced epidermal lipid synthesis, as well as reduced expression of the sodium-hydrogen antiporter, type 1 (NHE1). Among these many changes, much attention has been paid to the epidermal permeability barrier, because of its dominant role in regulating these metabolic responses.

Further, there tends to be dryness and itching as the epidermal barrier function deteriorates with aging, especially in winter, due to a steeper gradient of transepidermal water loss (TEWL), among other factors. An additional cause of dry skin as the population ages results from the side effects of widely-prescribed, orally-administered hypolipodemic drugs (e.g., statins), which may result in epidermal cholesterol deficiency and a barrier abnormality.

In view of the aging world population, there continues to be an unmet need for ameliorating the multiple negative effects of skin aging, particularly by restoring and maintaining the epidermal permeability barrier. Applications include treatments for chronologically- and photo-aged skin, diabetic skin, dry skin in aged and non-aged skin, ‘winter itch,’ inflamed skin in aged and non-aged skin, and glucocorticoid (GC)-treated skin. Notably, diabetic and GC-treated skin mimics aged skin, including both abnormally dry skin and impaired permeability barrier function. In addition, millions of individuals world-wide are receiving oral statins or other hypolipidemic drugs for the treatment of hyperlipidemia, including hyperlipidemia in aged humans. These drugs can aggravate pre-existing skin conditions of the aged, and provoke all of the problems of aged skin in otherwise normal skin. Disclosed herein, inter alia, are solutions to these and other problems in the art.

### BRIEF SUMMARY OF THE INVENTION

The present compositions and methods meet these needs. Because epidermal permeability barrier requirements regulate epidermal proliferation, differentiation, lipid production, as well as cutaneous innate immunity, strategies that improve barrier function by enhancing epidermal proliferation, differentiation and/or lipid production, while also reducing stratum corneum (SC) pH should prove useful for preventing and/or treating functional abnormalities, including the impaired permeability barrier homeostasis in aged skin.

We have now discovered that 1) hesperidin alone or 2) hesperidin combined with A) a triple-lipid barrier mixture that is cholesterol-dominant or with B) cholesterol alone, and formulated at a low pH; can successfully treat dry or inflamed or aged skin, and reverse or prevent the side effects that accompany diabetes or systemic or topical GC treatment, and reverse or prevent the side effects that accompany hypolipodemic drug treatment. In all of these situations, epidermal barrier function is compromised due to decreased differentiation, proliferation, impaired lipid secretion, or an elevated skin surface pH.

One aspect of the invention is directed to a topical composition providing desirable restorative effects on the epidermal permeability barrier in aged skin, as provided herein.

It has now been discovered that, although the combination of barrier lipids should be ceramide-dominant for diseased skin, with an optimal mole ratio of 1:3:1 cholesterol:ceramide:free fatty acids, for aged skin the combination of barrier lipids instead should be cholesterol-dominant, with an optimal mole ratio of 3:1:1 cholesterol:ceramide:free fatty acids.

Further, it has now been discovered that the pH of the applied composition should be acidic (i.e. pH<7), within an appropriate pH range that corrects the pH abnormality in aged skin, without causing irritation.

Thus, one aspect of the invention is directed to a topical composition, the composition comprising a) about 1 to about 50 wt % of cholesterol; b) about 0.01 to about 5 wt % of an acidic buffer; and c) about 0.001 to about 50 wt % of hesperidin; where the buffer content is sufficient to maintain the pH of the topical composition at 3 or above, but below 5.5. The lipid weight percent ranges from about 0.01% to about 5% of the formulation.

Another aspect of the invention is directed to a topical composition, the composition comprising a) a mixture of barrier lipids selected from the group consisting of cholesterol, at least one ceramide or synthetic ceramide, at least one free fatty acid, and mixtures of two or more thereof, in a total of about 1 to about 50 wt %; b) about 0.1 to about 10 wt % of a skin conditioner; c) about 0.001 to about 5 wt % of a chelating agent; d) about 1 to about 30 wt % of a humectant; e) about 0.5 to about 20 wt % of an emulsifier; f) one or more emollients in a total of about 1 to about 30 wt %; g) one or more preservatives in a total of about 0.1 to about 10 wt %; h) about 0.01 to about 5 wt % of an acid buffer; i) one or more encapsulation aids in a total of about 1 to about 25 wt %; j) about 0.01 to about 3 wt % of an aqueous phase thickener; and k) between 0.001 to about 50 wt % of hesperidin. In embodiments, the mixture of barrier lipids consists of cholesterol, hydroxypropyl bispalmitamide MEA (Synthetic ceramide; PC-104) and conjugated linoleic acid (CLA) at a 3:1:1 molar ratio. In embodiments, the skin conditioner comprises dimethicone, the chelating agent comprises disodium EDTA, the humectant comprises glycerin, and the acid buffer comprises citric acid. In embodiments, the emulsifier comprises glyceryl stearate PEG-100. In embodiments, the one or more additional emollients comprise petrolatum and squalane. In embodiments, the one or more preservatives comprise phenoxyethanol and sorbic acid. In embodiments, the one or more encapsulation aids comprise corn syrup solids and euphorbia cerifera (candelilla) wax. In embodiments, the aqueous phase thickener comprises xanthan gum. In a preferred embodiment the pH of the topical composition is ≧3 up to 5.5. In a preferred embodiment the molar ratio of cholesterol:ceramide:free fatty acid is 3:1:1. In embodiments, the cholesterol is present in about 0.1 to about 10 wt %. In embodiments, the ceramide or mixture of ceramides is present in about 0.1 to about 10 wt %. In embodiments, the free fatty acid or mixture of free fatty acids is present in about 0.01 to about 6 wt %.

One aspect of the invention is directed to a topical composition, the composition consisting of a) about 1 to about 50 wt % of a mixture of barrier lipids consisting of cholesterol, one or more ceramides, and one or more free fatty acids; b) about 0.1 to about 10 wt % of a skin conditioner; c) about 0.001 to about 5 wt % of a chelating agent; d) about 1 to about 30 wt % of a humectant; e) about 0.5 to about 20 wt % of glyceryl stearate PEG-100 emulsifier; f) about 1 to about 30 wt % of a mixture of emollients consisting of petrolatum and squalane; g) about 0.1 to about 10 wt % of a mixture of preservatives consisting of phenoxyethanol and sorbic acid; h) about 0.01 to about 5 wt % of citric acid; i) about 1 to about 25 wt % of a mixture of encapsulation aids consisting of corn syrup solids and euphorbia cerifera (candelilla) wax; j) about 0.01 to about 3 wt % of an aqueous phase thickener; k) about 0.001 to about 50 wt % of hesperidin; and l) water.

Another aspect of the invention is directed to composition comprising a) a mixture of barrier lipids selected from the group consisting of cholesterol, ceramides, free fatty acids and mixtures of two or more thereof, in a total of about 1 to about 50 wt %; b) about 0.001 to about 50 wt % of hesperidin; c) about 0.01 to about 5 wt % of an acid buffer; and d) about 0.001 to about 5% of at least one topical glucocorticoid (either separately or as the vehicle for the topical steroid).

One aspect of the invention is directed to a method of treating or preventing inflammation by administering an effective amount of the above anti-inflammatory composition to at least a portion of affected skin in need thereof.

A further aspect of the invention is directed to a method of treating aged skin, comprising administering one of the topical compositions described above to at least a portion of aged skin, in an amount effective to relieve or reverse a clinical indication of skin aging. Clinical indication of skin aging can be selected from the group consisting of eczema, xerosis, xerotic eczema, winter itch, nummular eczema, seborrheic dermatitis, occupational dermatitis, hand eczema, and stasis dermatitis.

Another aspect of the invention is directed to a method of preventing or reversing epidermal side effects of systemic or topical glucocorticoid treatment, comprising treating at least a portion of affected skin with one of the topical compositions described above, in an amount effective to prevent or reverse an epidermal clinical indication of glucocorticoid side effects. Epidermal clinical indications of glucocorticoid side effects can be selected from the group consisting of inflammation (steroid rosacea and/or peri-oral dermatitis), tachyphylaxis, disease flares, and rebound flares.

Yet another aspect of the invention is directed to a method for the treatment of the dry skin or abnormal epidermal barrier function associated with the administration of oral hypolipodemic agents, particularly statins, comprising one of the topical compositions described above to at least a portion of affected skin of a subject, in an amount effective to relieve or reverse clinical indications of hypolipodemic drug treatment. In embodiments, the subject is taking a hypolipodemic agent, such as simvastatin or pravastatin, or is expected to receive such treatment.

### BRIEF DESCRIPTION OF THE DRAWINGS

FIG. 1 shows a chart displaying inherited abnormalities in acidifying mechanisms which activate kallikreins (KLKs), comprising multiple ways by which pH functions in inflammatory dermatoses.

FIGS. 2A-2C displays the results of treatment of murine skin with a composition of the invention, as described herein. FIG. 2A displays a chart of changes in basal transepidermal water loss; FIG. 2B displays a chart of the changes in kinetics of barrier recovery rates, and FIG. 2C displays the skin surface pH.

FIGS. 3A-3C. The ventral (flexor) surface represents chronologically-aged (CA) skin with little photo-aging (PA), while the extensor surface reflects substantial PA, superimposed on CA. The visual results of the topical composition as described herein on both chronologically (CA) and photoaged (PA) skin were striking—signs of both CA and PA, such as xerotic scaling, atrophy (skin thinning) and ecchymoses, largely disappeared in sites treated with the hesperidin-containing, barrier repair formulation. These grossly apparent changes were paralleled by marked improvements in epidermal function, including a striking improvement in stratum corneum hydration (FIG. 3B), as well as moderate benefits for basal barrier function (FIG. 3A; p<0.1). But most striking were the benefits for PA skin, reflected by changes in cutaneous resonance running time (CRRT) (FIG. 3C; p<0.02).

### DETAILED DESCRIPTION

The permeability barrier in aged epidermis has been largely overlooked, because earlier functional studies described few if any abnormalities in aged skin. Yet, the permeation of a variety of molecules is altered across aged epidermis in vivo. Moreover, aged SC displays a global reduction in lipids, with reduced numbers of extracellular bilayers, indicative of lower reserve barrier capacity. Although an aged permeability barrier might function adequately under basal conditions, when acutely challenged, both barrier integrity and the kinetics of barrier recovery are impaired in both aged human and aged murine skin. The pH of skin tends to rise with age, favoring dermal inflammation and a predilection to infections. Further, the barrier abnormality after acute challenges to the epidermis of aged mice is associated with a paucity of lamellar body material at the stratum granulosum-SC interface, and a delay in the return of stainable lipids to the SC interstices. A global deficiency in the synthesis of all 3 physiologic lipids with a further reduction in cholesterologenesis accounts for the barrier abnormality in aged human epidermis. Such reductions in cholesterol biosynthesis begin at about age 50 in the human population.

Epidermal lipid synthesis is required for formation and maintenance of the epidermal permeability barrier. Synthesis of the three key barrier-related lipids, cholesterol, ceramides and free fatty acids, requires their respective rate-limiting enzymes, HMGCoA, SPT1, ACC, and FAS. Basal mRNA levels for all three key lipid synthetic enzymes were observed to be lower in aged epidermis as compared with young epidermis, consistent with the concept that lower lipid synthesis rates in aged epidermis reflect the reduced expression of their synthetic enzymes.

Although it was anticipated that topical applications of any one of the barrier lipids to aged skin would assist in the regeneration of the epidermal barrier layer, it has now been discovered that application to aged skin of the individual barrier lipids, rather than a combination of all 3 barrier lipids, actually compromises epidermal permeability barrier function. A combination of all three barrier lipids together, in an optimized ratio, is required to optimally improve barrier function.

This invention is based, at least in part, on the unexpected discovery that a topical composition containing hesperidin in combination with the cholesterol-dominant, triple-lipid mixture, formulated at an acidic pH, regulates and improves aging skin conditions. As mentioned above, the principal processes of skin aging take place both in the upper layers of skin (i.e., the epidermis) and in the dermis. A composition of this invention that regulates and improves the conditions of the epidermis, also improves the dermis by enhancing the nourishment of dermal tissues and other cellular components in the deeper layers of the skin.

### I. DEFINITIONS

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

An “effective amount” is an amount sufficient to accomplish a stated purpose (e.g. achieve the effect for which it is administered, treat a disease, change enzyme activity, increase enzyme activity, reduce protein function, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug or prodrug is an amount of a drug or prodrug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “associated” or “associated with” in the context of a substance or substance activity or function associated with a disease (e.g. dry skin, aged skin, or abnormal epidermal barrier function) means that the disease is caused by (in whole or in part), or a symptom of the disease is caused by (in whole or in part) the substance or substance activity or function. As used herein, what is described as being associated with a disease (e.g, dry skin, aged skin, or abnormal epidermal barrier function) if a causative agent, could be a target for treatment of the disease.

“Control” or “control experiment” or “standard control” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

“Contacting” is used in accordance with its plain ordinary meaning and refers to the process of allowing at least two distinct species (e.g. chemical compounds including biomolecules, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a protein or enzyme. In some embodiments contacting includes allowing a compound described herein to interact with a protein.

“Patient” or “subject in need thereof” or “subject” refers to a living organism suffering from or prone to a disease or condition that can be treated by administration of a compound or pharmaceutical composition or by a method, as provided herein. Non-limiting examples include humans, other mammals, bovines, rats, mice, dogs, monkeys, goat, sheep, cows, deer, and other non-mammalian animals. In some embodiments, a patient is human. In some embodiments, a subject is human.

“Disease” or “condition” refer to a state of being or health status of a patient or subject capable of being treated with a compound, pharmaceutical composition, or method provided herein. In embodiments, the disease is dry skin. In embodiments, the disease is aged skin. In embodiments, the disease is dry skin, aged skin, or abnormal epidermal barrier function.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it.

As used herein, the term “administering” means topical contact or subcutaneous administration, or the implantation of a slow-release device, e.g., a mini-osmotic pump, to a subject. The compositions of the present invention can be delivered by transdermally, by a topical route, formulated as applicator sticks, solutions, suspensions, emulsions, gels, creams, ointments, pastes, jellies, paints, powders, and aerosols. Liquid form preparations include solutions, suspensions, and emulsions, for example, water or water/propylene glycol solutions. The compositions of the present invention may additionally include components to provide sustained release and/or comfort. By “co-administer” it is meant that a composition described herein is administered at the same time, just prior to, or just after the administration of one or more additional therapies (e.g. glucocorticoid treatment, immunomodulator treatment, hypolipodemic agent). Co-administer may also include a composition described herein is administered 10 days prior or 10 days after administration of one or more additional therapies (e.g. glucocorticoid treatment, immunomodulator treatment, hypolipodemic agent. The compositions of the invention can be administered alone or can be co-administered to the patient. The compositions described herein can also be formulated in combination with one or more additional active ingredients which can include any pharmaceutical agent such immune enhancers, immune suppressants, anti inflammatory agents and the like. Co-administration is meant to include simultaneous or sequential administration of the compound individually or in combination (more than one compound or agent).

Pharmaceutical compositions provided by the present invention include compositions wherein the active ingredient (e.g. compounds described herein, including embodiments or examples) may be contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., reducing, eliminating, or slowing the progression of disease symptoms. Determination of a therapeutically effective amount of a compound of the invention is well within the capabilities of those skilled in the art, especially in light of the detailed disclosure herein.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents can be used in conjunction with the methods and compounds of Applicants' invention. Adjustment and manipulation of established dosages (e.g., frequency and duration) are well within the ability of those skilled in the art.

Dosages may be varied depending upon the requirements of the patient and the compound being employed. The dose administered to a patient, in the context of the present invention should be sufficient to affect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

“Analog” and “analogue” are used interchangeably and are used in accordance with their plain ordinary meaning within Chemistry and Biology and refers to a chemical compound that is structurally similar to another compound (i.e., a so-called “reference” compound (e.g., hesperidin)) but differs in composition, e.g., in the replacement of one atom by an atom of a different element, or in the presence of a particular functional group, or the replacement of one functional group by another functional group, or the absolute stereochemistry of one or more chiral centers of the reference compound, including isomers thereof. Accordingly, an analog is a compound that is similar or comparable in function and appearance but not in structure or origin to a reference compound. In embodiments, an analog has a similar function to the reference compound (e.g., as measured in one or more assays, such as the effect of hesperidin on aged skin).

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value.

As used herein the term “treating” is defined as administration of a composition to a subject with the purpose to cure, alleviate, relieve, remedy, prevent, or ameliorate a disorder, the symptom of the disorder, the disease state secondary to the disorder, or the predisposition toward the disorder. An “effective amount” is an amount of the composition that is capable of producing a medically desirable result, e.g., as described above, in a treated subject.

When the terms “prevent”, “preventing”, and “prevention” are used herein in connection with a given treatment for a given condition, they mean that the treated patient either does not develop a clinically observable level of the condition at all, or develops it more slowly and/or to a lesser degree than he/she would have absent the treatment. These terms are not limited solely to a situation in which the patient experiences no aspect of the condition whatsoever. For example, a treatment will be said to have “prevented” the condition if it is given during exposure of a patient to a stimulus that would have been expected to produce a given manifestation of the condition, and results in the patient's experiencing fewer and/or milder symptoms of the condition than otherwise expected. A treatment can “prevent” infection by resulting the patient's displaying only mild overt symptoms of the infection; it does not imply that there must have been no penetration of any cell by the infecting microorganism. The treated patient either does not develop a clinically observable level of the condition at all, or develops it more slowly and/or to a lesser degree than a non-treated patient exposed to same condition-inducing stimulus as the treated patient, where the susceptibility of the non-treated patient to the condition is the same as the treated patient's susceptibility prior to treatment.

As used herein, “identifying” a subject in need of such treatment can be in the judgment of a subject or a health care professional and can be subjective (e.g. opinion) or objective (e.g. measurable by a test or diagnostic method).

The term “cholesterol” refers to (3β)-choles-5-en-3-ol, or any salt form or isomer thereof.

The term “hesperidin” refers to the compound (2S)-5-hydroxy-2-(3-hydroxy-4-methoxyphenyl)-7-[(2S,3R,4S,5S,6R)-3,4,5-trihydroxy-6-[[(2R,3R,4R,5R,6S)-3,4,5-trihydroxy-6-methyloxan-2-yl]oxymethyl]oxan-2-yl]oxy-2,3-dihydrochromen-4-one, or any salt, isomer, or salt thereof. In embodiments, hesperidin has the structure:

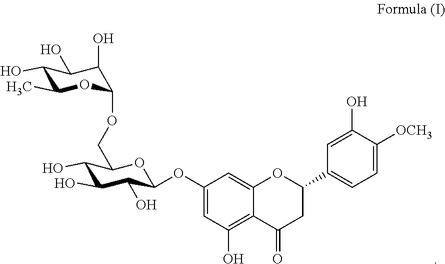

Hesperidin may be referred to as a flavanone glycoside including the flavone hesperetin bound to the disaccharide rutinose, and may be found in citrus fruits and other natural sources. Its name is derived from “hesperidium”, for fruit produced by citrus trees, and the compound is found in oranges, lemons, limes and pummelo fruits, among others. Peppermint leaves also contain hesperidin. The aglycone form may be referred to as hesperetin; thus an alternative name for hesperidin is hesperetin 7-rutinoside. A hesperidin analog is a compound functionally similar to hesperidin. In embodiments, the hesperidin analog is chemically similar to hesperidin but differing in one or more atoms, wherein the analog includes one or more functions or effects of hesperidin on aged skin.

The term “dry skin” is used in accordance with its plain ordinary meaning and refers to skin wherein there is a reduced amount of water in the epidermis. For example, dry skin may be associated with greater levels of transepidermal water loss (TEWL). Additionally, dry skin may have reduced levels of at least one of cholesterol, ceramides, glycerol, filaggrin, and natural moisturizing molecules (e.g., urocanic acid). Dry skin may result from use of systemic hypolipidemic drugs (e.g., statins), glucocorticoid treatment, or immunomodulator treatment. Symptoms associated with dry skin include the visible peeling of the outer skin layer, itching, and skin cracking.

The term “abnormal epidermal barrier function” is used in accordance with its plain ordinary meaning and refers to aberrant function in the epidermis (e.g., due to decreased differentiation, proliferation, impaired lipid secretion, or an elevated skin surface pH (e.g., pH>5.5) relative to a healthy control (e.g., population average, skin at another location on the same subject as the aged skin)). Abnormal epidermal barrier function may be associated with the use of systemic hypolipidemic drugs (e.g., statins), glucocorticoid treatment, or immunomodulator treatment. Abnormal epidermal barrier function may result in visible or tactile wrinkles or discontinuities in skin associated with skin aging, e.g., visible and/or tactile scaling, wrinkles or discontinuities in skin texture or color.

The term “aged skin” is used in accordance with its plain ordinary meaning and refers to chronologically- and/or photo-aged skin, typically characterized by abnormal epidermal barrier function or a flawed permeability barrier (e.g., characterized by a thinner epidermis with reduced epidermal proliferation, abnormal differentiation, impaired lipid synthesis and/or an elevated skin surface pH). Aged skin (e.g., chronologically- or photo-) may display abnormal epidermal barrier function resulting in impaired barrier function, reduced antimicrobial defense, and/or reduced cutaneous wound healing. Aged skin (e.g., chronologically- or photo-), may display abnormal epidermal barrier function such as reduced stratum corneum hydration, reduced stratum corneum cohesion, increased abnormal desquamation, increased skin scaling, increased occurrence of skin infections, increased susceptibility to skin infections, increased susceptibility of inflammatory dermatoses, and increased occurrence of inflammatory dermatoses (e.g., increased relative to normal, average, or healthy skin of a control (e.g., population average, skin at another location on the same subject as the aged skin)). Clinical indications of aged skin may include eczema, xerosis, xerotic eczema, winter itch, nummular eczema, seborrheic dermatitis, occupational dermatitis, hand eczema, or stasis dermatitis.

The term “photo-aged skin” is used in accordance with its plain ordinary meaning and refers to skin displaying abnormal epidermal barrier function as a result of chronic exposure to ultraviolet radiation (e.g., 300-400 nm).

The term “barrier lipids” is used in accordance with its plain ordinary meaning and refers to epidermal lipids of both sebaceous and keratinocyte origin. Non-limiting examples include non-polar lipids, mainly triglycerides, wax esters, squalane, fatty acids, free fatty acids, ceramides cholesterol, cholesterol esters and diglycerides. In embodiments, barrier lipids are ceramides, cholesterol, and/or free fatty acids.

The term “ceramide” is used in accordance with its plain ordinary meaning and refers to sphingosine (e.g., 2-amino-4-octadecene-1,3-diol) and a fatty acid. In embodiments, the ceramide is a pseudoceramide (i.e., synthetic) as described in Uchida, et al (Journal of Dermatological Science, Volume 51, 37-43) hereby expressly incorporated by reference in its entirety for any purpose. Non-limiting examples of a ceramide include myristoyl/palmitoyl oxostearamide/arachamide MEA, myristoyl/palmitoyl oxostearamide/arachamide, N-(Ethyl dihydrogenphosphate)-2-hexyl-3-oxo-decanamide, N-Ethanol-2-mirystyl-3-oxo-staramide, ceramide PC-102 (Hydroxypropyl Bislauramide MEA), ceramide PC-104 (Hydroxypropyl Bispalmitamide MEA), and ceramide PC-108 (Hydroxypropyl Bisstearamide MEA). In embodiments, the ceramide is a synthetic ceramide. In embodiments, the ceramide is N-(3-hexadecyloxy-2-hydroxypropyl)-N-2-hydroxyethylhexadecanamide (PC-104) or hydroxyethyl)-2-pentadecanolylhexadecanamide (Bio391). In embodiments, the ceramide is myristoyl/palmitoyl oxostearamide/arachamide MEA.

The term “fatty acid” or “free fatty acid is used in accordance with its plain ordinary meaning and refers to a carboxylic acid with a long (e.g., C₄-C₃₀) aliphatic chain, which may be saturated or unsaturated, including all positional and geometrical isomers. For example, a fatty acid may be lauric acid, palmitic acid, stearic acid, oleic acid, linoleic acid, linolenic acid, arachidonic acid, or conjugated linoleic acid.

The term “skin conditioner” is used in accordance with its plain ordinary meaning and refers to a an agent that attracts or retains moisture in the skin (e.g, a silicon-based polymer). Non-limiting examples of a skin conditioner include polydimethylsiloxane (i.e. dimethicone) and dimethicone copolyol.

The term “chelating agent” is used in accordance with its plain ordinary meaning and refers to chemical compounds that coordinate with metal atoms. Typically a chelating agent forms two or more separate coordinate bonds between a polydentate ligand and a single central atom (e.g., a metal atom). Non-limiting examples of a chelating agent include disodium ethylenediaminetetraacetic acid (EDTA), tetrasodium EDTA and tetrahydroxypropyl ethylenediamine.

The term “humectant” is used in accordance with its plain ordinary meaning and refers to a hygroscopic agent which promotes retention of moisture. Non-limiting examples of a humectant include glycerin, urea, aloe vera, honey, and sorbitol, xylitol, maltitol.

The term “emulsifier” is used in accordance with its plain ordinary meaning and refers to an agent that stabilizes a mixture of two or more compounds that are typically immiscible. Non-limiting examples of an emulsifier includes emulsifying wax, cetearyl alcohol, polysorbate 20, ceteareth 20, glycerl stearate PEG-100 and modified food starches (e.g., corn syrup solids).

The term “emollient” is used in accordance with its plain ordinary meaning and refers to compounds which increase the water content of the epidermis by reducing evaporation. Non-limiting examples include squalane and petrolatum.

The term “preservative” is used in accordance with its plain ordinary meaning and refers to agents intended to prevent microbial growth and spoiling of a composition described herein. Non-limiting examples of preservatives include sorbic acid, sodium sorbate, potassium sorbate, calcium sorbate, and glycol ethers (e.g., dimethoxyethane, phenoxyethanol, or 2-butoxyethyl acetate).

The term “acid buffer” is used in accordance with its plain ordinary meaning and refers to a weak acid and its conjugate base used to control the pH (e.g., wherein the pH is less than 7). Non-limiting examples include citric acid.

The term “encapsulation aids” is used in accordance with its plain ordinary meaning and refers to agents used control the release of the active substance. Non-limiting examples include modified food starch (e.g., corn syrup solids), aliginate, and euphorbia cerifera (candelilla) wax.

The term “aqueous phase thickener” is used in accordance with its plain ordinary meaning and refers to a rheology modifier (e.g., an agent capable of modulating (e.g., increasing) the viscosity of a liquid without substantially changing other properties of the liquid).

The term “glucocorticoid” is used in accordance with its plain ordinary meaning and refers to a corticosteroid (e.g., that binds to the glucocorticoid receptor). Non-limiting examples of a glucocorticoid include clobetasol propionate, betamethasone dipropionate, halobetasol propionate, diflorasone diacetate, fluocinonide, halcinonide, amcinonide, desoximetasone, triamcinolone acetonide, mometasone furoate, fluticasone propionate, halometasone, fluocinolone acetonide, hydrocortisone valerate, hydrocortisone butyrate, flurandrenolide, triamcinolone acetonide, mometasone furoate, fluticasone propionate, desonide, fluocinolone acetonide, hydrocortisone valerate, alclometasone dipropionate, Triamcinolone acetonide, fluocinolone acetonide, desonide, and hydrocortisone.

Examples of skin conditioner, chelating agent, emollient, humectant, emulsifier, preservative, acid buffer, encapsulation aids, aqueous phase thickener are described in Harry's Cosmeticology, 7th Ed., Harry & Wilkinson (Hill Publishers, London 1982); in Pharmaceutical Dosage Forms—Disperse Systems; Lieberman, Rieger & Banker, Vols. 1 (1988) & 2 (1989); Marcel Decker, Inc.; in The Chemistry and Manufacture of Cosmetics, 2nd. Ed., deNavarre (Van Nostrand 1962-1965); and in The Handbook of Cosmetic Science and Technology, 1st Ed. Knowlton & Pearce (Elsevier 1993) can also be used in the present invention.

The term “glyceryl stearate PEG-100” refers to a water soluble ester with and an average of number of ethylene oxide monomers in the polyethylene chain around 100. For example, glyceryl stearate PEG-100 is an off-white, solid ester of polyethylene glycol (a binder and a softener) and stearic acid.

The term “glucocorticoid side effects” is used in accordance with its plain ordinary meaning and refers to inflammation (e.g., steroid rosacea and/or peri-oral dermatitis), tachyphylaxis, disease flares, or rebound flares caused by glucocorticoid treatment, or other unintended effects of using glucocorticoids.

The term “citrus bioflavonoid” is used in accordance with its plain ordinary meaning and refers to compounds present in citrus fruits (e.g., lemons, limes, oranges, grapefruits, and tangerines). Citrus bioflavonoids may be bioflavonoids present in citrus plants (e.g., citrus fruit, leaves, roots, etc.). Citrus bioflavonoids include citrus flavanones, which may optionally be glycosylated (e.g., by a disaccharide) to a citrus flavanone glycoside, which is also included in citrus bioflavonoids (e.g., hesperidin). Examples of citrus bioflavonoids include apigenin, hesperidin, hesperitin, naringenin, naringin, narirutin, nobiletin, tangeretin, and tangeritin. Citrus bioflavonoids include hesperidin. Citrus bioflavonoids include hesperitin. Citrus bioflavonoids may include eriodictyol, homoeriodictyol, and luteolin. Citrus bioflavonoids include citrus flavones, citrus flavonol, citrus flavanones, citrus flavanonols, citrus flavans, and citrus flavanols.

The term “immunomodulator” is used in accordance with its plain ordinary meaning and refers to compounds that modulate the immune system. Examples of immunomodulators include pimecrolimus and/or tacrolimus. In embodiments, the immunomodulator is a topical calcineurin inhibitor (e.g., pimecrolimus and/or tacrolimus). Examples of additional topical calcineurin inhibitors can be found in Breuer et al. (Am. J. Clin. Dermatol. 2005; 6(2):65-77) and is incorporated herein in its entirety.

### II. COMPOSITIONS

In an aspect is provided a topical composition, the composition including a mixture of barrier lipids selected from the group consisting of cholesterol, at least one ceramide, at least one free fatty acid, and mixtures of two or more thereof and a citrus bioflavonoid (e.g., hesperidin, or an analog, pharmaceutically acceptable salt, or prodrug thereof). In embodiments is provided a topical composition, the composition including a mixture of barrier lipids selected from the group consisting of cholesterol, at least one ceramide, at least one free fatty acid, and mixtures of two or more thereof and hesperidin or an analog, pharmaceutically acceptable salt, or prodrug thereof. In embodiments is provided a topical composition, the composition including a mixture of barrier lipids selected from the group consisting of cholesterol, at least one ceramide, at least one free fatty acid, and mixtures of two or more thereof and hesperidin. In embodiments, the composition is capable of treating aged skin.

In an aspect is provided a topical composition for treating dry skin or skin suffering an abnormal epidermal barrier function, the composition including a mixture of barrier lipids selected from the group consisting of cholesterol, at least one ceramide, at least one free fatty acid, and mixtures of two or more thereof and a citrus bioflavonoid (e.g., hesperidin, or an analog, pharmaceutically acceptable salt, or prodrug thereof). In embodiments is provided a topical composition for treating dry skin or skin suffering an abnormal epidermal barrier function, the composition including a mixture of barrier lipids selected from the group consisting of cholesterol, at least one ceramide, at least one free fatty acid, and mixtures of two or more thereof and hesperidin or an analog, pharmaceutically acceptable salt, or prodrug thereof. In embodiments is provided a topical composition for treating dry skin or skin suffering an abnormal epidermal barrier function, the composition including a mixture of barrier lipids selected from the group consisting of cholesterol, at least one ceramide, at least one free fatty acid, and mixtures of two or more thereof and hesperidin.

In an aspect is provided a topical composition for treating aged skin, the composition including a mixture of barrier lipids selected from the group consisting of cholesterol, at least one ceramide, at least one free fatty acid, and mixtures of two or more thereof and citrus bioflavonoid (e.g., hesperidin, or an analog, pharmaceutically acceptable salt, or prodrug thereof). In embodiments is provided a topical composition for treating aged skin, the composition including a mixture of barrier lipids selected from the group consisting of cholesterol, at least one ceramide, at least one free fatty acid, and mixtures of two or more thereof and hesperidin or an analog, pharmaceutically acceptable salt, or prodrug thereof. In embodiments, the topical composition includes a mixture of barrier lipids selected from the group consisting of cholesterol, at least one ceramide, at least one free fatty acid, and mixtures of two or more thereof and hesperidin.

In embodiments, the topical composition further comprises a skin conditioner. In embodiments, the topical composition further comprises a chelating agent. In embodiments, the topical composition further comprises a humectant. In embodiments, the topical composition further comprises an emulsifier. In embodiments, the topical composition further comprises one or more emollients. In embodiments, the topical composition further comprises one or more preservatives. In embodiments, the topical composition further comprises an acid buffer. In embodiments, the topical composition further comprises one or more encapsulation aids. In embodiments, the topical composition further comprises an aqueous phase thickener. In embodiments, the tropical composition is capable of treating aged skin (e.g., a symptom of aged skin, photo-aged skin, chronologically-aged skin).

One aspect of the invention is directed to a composition, the composition including a) about 0.1 to about 50 wt % of cholesterol, preferably about 1 to about 20 wt %, more preferably about 2 to about 5 wt %; b) about 0.01 to about 5 wt % of an acid buffer, preferably about 0.25 to about 3 wt %, more preferably about 0.5 to about 2 wt %; and c) about 0.001 to about 50 wt % of hesperidin, or an analog, pharmaceutically acceptable salt, or prodrug thereof, preferably about 1 to about 10 wt %, more preferably about 2 to about 5 wt %; where the buffer content is sufficient to maintain the pH of the topical composition at about 3 to about 5.5. One aspect of the invention is directed to a topical composition for treating dry skin or skin suffering an abnormal epidermal barrier function, the composition including a) about 0.1 to about 50 wt % of cholesterol, preferably about 1 to about 20 wt %, more preferably about 2 to about 5 wt %; b) about 0.01 to about 5 wt % of an acid buffer, preferably about 0.25 to about 3 wt %, more preferably about 0.5 to about 2 wt %; and c) about 0.001 to about 50 wt % of a citrus bioflavonoid (e.g., hesperidin, or an analog, pharmaceutically acceptable salt, or prodrug thereof), preferably about 1 to about 10 wt %, more preferably about 2 to about 5 wt %; where the buffer content is sufficient to maintain the pH of the topical composition from about 3 to about 5.5. In embodiments, the citrus bioflavonoid is hesperidin.

One aspect of the invention is directed to a composition, the composition including a) about 0.1 to about 50 wt % of cholesterol, preferably about 1 to about 20 wt %, more preferably about 2 to about 5 wt %; b) about 0.01 to about 5 wt % of an acid buffer, preferably about 0.25 to about 3 wt %, more preferably about 0.5 to about 2 wt %; and c) about 0.001 to about 50 wt % of hesperidin, or an analog, pharmaceutically acceptable salt, or prodrug thereof, preferably about 1 to about 10 wt %, more preferably about 2 to about 5 wt %; where the buffer content is sufficient to maintain the pH of the topical composition at about 3 to about 5.5. One aspect of the invention is directed to a topical composition for treating dry skin or skin suffering an abnormal epidermal barrier function, the composition including a) about 0.1 to about 50 wt % of cholesterol, preferably about 1 to about 20 wt %, more preferably about 2 to about 5 wt %; b) about 0.01 to about 5 wt % of an acid buffer, preferably about 0.25 to about 3 wt %, more preferably about 0.5 to about 2 wt %; and c) about 0.001 to about 50 wt % of hesperidin, preferably about 1 to about 10 wt %, more preferably about 2 to about 5 wt %; where the buffer content is sufficient to maintain the pH of the topical composition at about 3 to about 5.5.

One aspect of the invention is directed to a composition, the composition including a) 0.1 to 50 wt % of cholesterol, preferably 1 to 20 wt %, more preferably 2 to 5 wt %; b) 0.01 to 5 wt % of an acid buffer, preferably 0.25 to 3 wt %, more preferably 0.5 to 2 wt %; and c) 0.001 to 50 wt % of hesperidin, or an analog, pharmaceutically acceptable salt, or prodrug thereof, preferably 1 to 10 wt %, more preferably 2 to 5 wt %; where the buffer content is sufficient to maintain the pH of the topical composition at 3 to 5.5. One aspect of the invention is directed to a topical composition for treating dry skin or skin suffering an abnormal epidermal barrier function, the composition including a) 0.1 to 50 wt % of cholesterol, preferably 1 to 20 wt %, more preferably 2 to 5 wt %; b) 0.01 to 5 wt % of an acid buffer, preferably 0.25 to 3 wt %, more preferably 0.5 to 2 wt %; and c) 0.001 to 50 wt % of a citrus bioflavonoid (e.g., hesperidin, or an analog, pharmaceutically acceptable salt, or prodrug thereof), preferably 1 to 10 wt %, more preferably 2 to 5 wt %; where the buffer content is sufficient to maintain the pH of the topical composition at 3 to 5.5. In embodiments, the citrus bioflavonoid is hesperidin.

One aspect of the invention is directed to a composition, the composition including a) 0.1 to 50 wt % of cholesterol, preferably 1 to 20 wt %, more preferably 2 to 5 wt %; b) 0.01 to 5 wt % of an acid buffer, preferably 0.25 to 3 wt %, more preferably 0.5 to 2 wt %; and c) 0.001 to 50 wt % of hesperidin, or an analog, pharmaceutically acceptable salt, or prodrug thereof, preferably 1 to 10 wt %, more preferably 2 to 5 wt %; where the buffer content is sufficient to maintain the pH of the topical composition at 3 to 5.5. One aspect of the invention is directed to a topical composition for treating dry skin or skin suffering an abnormal epidermal barrier function, the composition including a) 0.1 to 50 wt % of cholesterol, preferably 1 to 20 wt %, more preferably 2 to 5 wt %; b) 0.01 to 5 wt % of an acid buffer, preferably 0.25 to 3 wt %, more preferably 0.5 to 2 wt %; and c) 0.001 to 50 wt % of hesperidin, preferably 1 to 10 wt %, more preferably 2 to 5 wt %; where the buffer content is sufficient to maintain the pH of the topical composition at 3 to 5.5.

In embodiments, the cholesterol is present in about 0.1 to about 50 wt %. In embodiments, the cholesterol is present in about 0.2, 0.3, 0.4, 0.5, 0.6, 0.7, 0.8, 0.9, 1, 1.1, 1.2, 1.3, 1.4, 1.5, 2, 3, 4, 5, 6, 7, 8, 9, 10, 11, 12, 13, 14, 15, 16, 17, 18, 19, 20, 21, 2, 23, 24, 25, 26, 27, 28, 29, 30, 31, 32, 33, 34, 35, 36, 37, 38, 39, 40, 41, 42, 43, 44, 45, 46, 47, 48, 49 or about 50 wt %. In embodiments, the cholesterol is present in 0.2, 0.3, 0.4, 0.5, 0.6, 0.7, 0.8, 0.9, 1, 1.1, 1.2, 1.3, 1.4, 1.5, 2, 3, 4, 5, 6, 7, 8, 9, 10, 11, 12, 13, 14, 15, 16, 17, 18, 19, 20, 21, 2, 23, 24, 25, 26, 27, 28, 29, 30, 31, 32, 33, 34, 35, 36, 37, 38, 39, 40, 41, 42, 43, 44, 45, 46, 47, 48, 49 or 50 wt %. In embodiments, the cholesterol is present in about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or about 3.0 wt %. In embodiments, the cholesterol is present in 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 wt %. In embodiments, the cholesterol is present in about 1.0 wt %. In embodiments, the cholesterol is present in about 0.5 to about 1.5 wt %. In embodiments, the cholesterol is present in about 0.8 to about 1.2 wt %. In embodiments, the cholesterol is present in about 0.9 to about 1.1 wt %. In embodiments, the cholesterol is present in 1.0 wt %. In embodiments, the cholesterol is present in 0.5 to 1.5 wt %. In embodiments, the cholesterol is present in 0.8 to 1.2 wt %. In embodiments, the cholesterol is present in 0.9 to 1.1 wt %.

In embodiments, the acid buffer is present in about 0.1 to about 50 wt %. In embodiments, the acid buffer is present in about 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 1, 2, 3, 4, 5, 6, 7, 8, 9, 10, 11, 12, 13, 14, 15, 16, 17, 18, 19, 20, 21, 2, 23, 24, 25, 26, 27, 28, 29, 30, 31, 32, 33, 34, 35, 36, 37, 38, 39, 40, 41, 42, 43, 44, 45, 46, 47, 48, 49 or about 50 wt %. In embodiments, the acid buffer is present in about 0.5 wt %. In embodiments, the acid buffer is present in about 0.1 to about 5 wt %. In embodiments, the acid buffer is present in about 0.1 to about 3 wt %. In embodiments, the acid buffer is present in about 0.1 to about 2 wt %. In embodiments, the acid buffer is present in about 0.1 to about 1.2 wt %. In embodiments, the acid buffer is present in about 0.4 to about 1.0 wt %.

In embodiments, the acid buffer is present in 0.1 to 50 wt %. In embodiments, the acid buffer is present in 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 1, 2, 3, 4, 5, 6, 7, 8, 9, 10, 11, 12, 13, 14, 15, 16, 17, 18, 19, 20, 21, 2, 23, 24, 25, 26, 27, 28, 29, 30, 31, 32, 33, 34, 35, 36, 37, 38, 39, 40, 41, 42, 43, 44, 45, 46, 47, 48, 49 or 50 wt %. In embodiments, the acid buffer is present in 0.5 wt %. In embodiments, the acid buffer is present in 0.1 to 5 wt %. In embodiments, the acid buffer is present in 0.1 to 3 wt %. In embodiments, the acid buffer is present in 0.1 to 2 wt %. In embodiments, the acid buffer is present in 0.1 to 1.2 wt %. In embodiments, the acid buffer is present in 0.4 to 1.0 wt %.

In embodiments, the citrus bioflavonoid (e.g., hesperidin, or an analog, pharmaceutically acceptable salt, or prodrug thereof) is present in about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, or about 50 wt %. In embodiments, the citrus bioflavonoid (e.g., hesperidin, or an analog, pharmaceutically acceptable salt, or prodrug thereof) is present in 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 4, 5, 6, 7, 8, 9, 10, 20, 30, 40, or 50 wt %. In embodiments, the citrus bioflavonoid (e.g., hesperidin, or an analog, pharmaceutically acceptable salt, or prodrug thereof) is present in about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or about 3.0 wt %. In embodiments, the citrus bioflavonoid (e.g., hesperidin, or an analog, pharmaceutically acceptable salt, or prodrug thereof) is present in 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 wt %. In embodiments, the citrus bioflavonoid (e.g., hesperidin, or an analog, pharmaceutically acceptable salt, or prodrug thereof) is present in about 0.5 to about 3.0 wt %. In embodiments, the citrus bioflavonoid (e.g., hesperidin, or an analog, pharmaceutically acceptable salt, or prodrug thereof) is present in about 1.0 to about 2.8 wt %. In embodiments, the citrus bioflavonoid (e.g., hesperidin, or an analog, pharmaceutically acceptable salt, or prodrug thereof) is present in about 1.5 to about 2.2 wt %. In embodiments, the citrus bioflavonoid (e.g., hesperidin, or an analog, pharmaceutically acceptable salt, or prodrug thereof) is present in about 1.8 to about 2.1 wt %. In embodiments, the citrus bioflavonoid (e.g., hesperidin, or an analog, pharmaceutically acceptable salt, or prodrug thereof) is present in about 1.9 to about 2.1 wt %. In embodiments, the citrus bioflavonoid (e.g., hesperidin, or an analog, pharmaceutically acceptable salt, or prodrug thereof) is present in 0.5 to 3.0 wt %. In embodiments, the citrus bioflavonoid (e.g., hesperidin, or an analog, pharmaceutically acceptable salt, or prodrug thereof) is present in 1.0 to 2.8 wt %. In embodiments, the citrus bioflavonoid (e.g., hesperidin, or an analog, pharmaceutically acceptable salt, or prodrug thereof) is present in 1.5 to 2.2 wt %. In embodiments, the citrus bioflavonoid (e.g., hesperidin, or an analog, pharmaceutically acceptable salt, or prodrug thereof) is present in 1.8 to 2.1 wt %. In embodiments, the citrus bioflavonoid (e.g., hesperidin, or an analog, pharmaceutically acceptable salt, or prodrug thereof) is present in 1.9 to 2.1 wt %. In embodiments, the citrus bioflavonoid (e.g., hesperidin, or an analog, pharmaceutically acceptable salt, or prodrug thereof) is present in about 2.0 wt %. In embodiments, the citrus bioflavonoid (e.g., hesperidin, or an analog, pharmaceutically acceptable salt, or prodrug thereof) is present in 2.0 wt %.

In embodiments, citrus bioflavonoid (e.g., hesperidin, or an analog, pharmaceutically acceptable salt, or prodrug thereof) is present in about 2 wt %. In embodiments, citrus bioflavonoid (e.g., hesperidin, or an analog, pharmaceutically acceptable salt, or prodrug thereof) is present in about 5 wt %. In embodiments citrus bioflavonoid (e.g., hesperidin, or an analog, pharmaceutically acceptable salt, or prodrug thereof) is present in about 10 wt %. In embodiments, citrus bioflavonoid (e.g., hesperidin, or an analog, pharmaceutically acceptable salt, or prodrug thereof) is present in about 15 wt %. In embodiments, citrus bioflavonoid (e.g., hesperidin, or an analog, pharmaceutically acceptable salt, or prodrug thereof) is present in about 20 wt %. In embodiments, citrus bioflavonoid (e.g., hesperidin, or an analog, pharmaceutically acceptable salt, or prodrug thereof) is present in about 25 wt %. In embodiments, citrus bioflavonoid (e.g., hesperidin, or an analog, pharmaceutically acceptable salt, or prodrug thereof) is present in about 30 wt %. In embodiments, citrus bioflavonoid (e.g., hesperidin, or an analog, pharmaceutically acceptable salt, or prodrug thereof) is present in about 35 wt %. In embodiments, citrus bioflavonoid (e.g., hesperidin, or an analog, pharmaceutically acceptable salt, or prodrug thereof) is present in 2 wt %. In embodiments, citrus bioflavonoid (e.g., hesperidin, or an analog, pharmaceutically acceptable salt, or prodrug thereof) is present in 5 wt %. In embodiments citrus bioflavonoid (e.g., hesperidin, or an analog, pharmaceutically acceptable salt, or prodrug thereof) is present in 10 wt %. In embodiments, citrus bioflavonoid (e.g., hesperidin, or an analog, pharmaceutically acceptable salt, or prodrug thereof) is present in 15 wt %. In embodiments, citrus bioflavonoid (e.g., hesperidin, or an analog, pharmaceutically acceptable salt, or prodrug thereof) is present in 20 wt %. In embodiments, citrus bioflavonoid (e.g., hesperidin, or an analog, pharmaceutically acceptable salt, or prodrug thereof) is present in 25 wt %. In embodiments, citrus bioflavonoid (e.g., hesperidin, or an analog, pharmaceutically acceptable salt, or prodrug thereof) is present in 30 wt %. In embodiments, citrus bioflavonoid (e.g., hesperidin, or an analog, pharmaceutically acceptable salt, or prodrug thereof) is present in 35 wt %. In embodiments, the citrus bioflavonoid (e.g., hesperidin, or an analog, pharmaceutically acceptable salt, or prodrug thereof) can be contained within its natural source, such as orange rind or peppermint oil, in an amount sufficient to provide the required wt % of the active citrus bioflavonoid (e.g., hesperidin, or an analog, pharmaceutically acceptable salt, or prodrug thereof). In embodiments, the citrus bioflavonoid is hesperidin.

In embodiments, the hesperidin is present in about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, or about 50 wt %. In embodiments, the hesperidin is present in 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 4, 5, 6, 7, 8, 9, 10, 20, 30, 40, or 50 wt %. In embodiments, the hesperidin is present in about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or about 3.0 wt %. In embodiments, the hesperidin is present in 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 wt %. In embodiments, the hesperidin is present in about 0.5 to about 3.0 wt %. In embodiments, the hesperidin is present in about 1.0 to about 2.8 wt %. In embodiments, the hesperidin is present in about 1.5 to about 2.2 wt %. In embodiments, the hesperidin is present in about 1.8 to about 2.1 wt %. In embodiments, the hesperidin is present in about 1.9 to about 2.1 wt %. In embodiments, the hesperidin is present in 0.5 to 3.0 wt %. In embodiments, the hesperidin is present in 1.0 to 2.8 wt %. In embodiments, the hesperidin is present in 1.5 to 2.2 wt %. In embodiments, the hesperidin is present in 1.8 to 2.1 wt %. In embodiments, the hesperidin is present in 1.9 to 2.1 wt %. In embodiments, the hesperidin is present in about 2.0 wt %. In embodiments, the hesperidin is present in 2.0 wt %.

In embodiments, hesperidin is present in about 5 wt %. In embodiments hesperidin is present in about 10 wt %. In embodiments, hesperidin is present in about 15 wt %. In embodiments, hesperidin is present in about 20 wt %. In embodiments, hesperidin is present in about 25 wt %. In embodiments, hesperidin is present in about 30 wt %. In embodiments, hesperidin is present in about 35 wt %. In embodiments, hesperidin is present in 2 wt %. In embodiments, hesperidin is present in 5 wt %. In embodiments hesperidin is present in 10 wt %. In embodiments, hesperidin is present in 15 wt %. In embodiments, hesperidin is present in 20 wt %. In embodiments, hesperidin is present in 25 wt %. In embodiments, hesperidin is present in 30 wt %. In embodiments, hesperidin is present in 35 wt %. In embodiments, the hesperidin can be contained within its natural source, such as orange rind or peppermint oil, in an amount sufficient to provide the required wt % of the active hesperidin.

In embodiments, the topical composition further comprises a skin conditioner, a chelating agent, a humectant, an emulsifier, one or more emollients, one or more preservatives, an acid buffer, one or more encapsulation aids, and an aqueous phase thickener. In embodiments, the topical composition further comprises a skin conditioner, a chelating agent, a humectant, an emulsifier, one or more emollients, one or more preservatives, an acid buffer, one or more encapsulation aids, or an aqueous phase thickener.

Another aspect of the invention is directed to a topical composition for treating aged skin, the composition including a) a mixture of barrier lipids selected from the group consisting of cholesterol, at least one ceramide, at least one free fatty acid, and mixtures of two or more thereof, in a total of about 1 to about 50 wt %, preferably about 1 to about 20 wt %, more preferably about 2 to about 10 wt %; b) about 0.1 to about 10 wt % of a skin conditioner, preferably about 1 to about 5 wt %, more preferably about 2 to about 4 wt %; c) about 0.001 to about 5 wt % of a chelating agent, preferably about 0.01 to about 2 wt %, more preferably about 0.1 to about 1 wt %; d) about 1 to about 30 wt % of a humectant, preferably about 3 to about 20 wt %, more preferably about 5 to about 10 wt %; e) about 0.5 to about 20 wt % of an emulsifier, preferably about 0.5 to about 10 wt %, more preferably about 1 to about 5 wt %; f) one or more emollients in a total of about 1 to about 30 wt %, preferably about 2 to about 20 wt %, more preferably about 4 to about 10 wt %; g) one or more preservatives in a total of about 0.1 to about 10 wt %, preferably about 0.5 to about 5 wt %, more preferably about 0.8 to about 1.5 wt %; h) about 0.01 to about 5 wt % of an acid buffer, preferably about 0.25 to about 3 wt %, more preferably about 0.5 to about 2 wt %; i) one or more encapsulation aids in a total of about 1 to about 25 wt %, preferably about 5 to about 15 wt %, more preferably about 8 to about 10 wt %; j) about 0.01 to about 3 wt % of an aqueous phase thickener, preferably about 0.1 to about 2 wt %, more preferably about 0.2 to about 1 wt %; and k) about 0.001 to about 50 wt % of citrus bioflavonoid (e.g., hesperidin, or an analog, pharmaceutically acceptable salt, or prodrug thereof), preferably about 1 to about 10 wt %, more preferably about 2 to about 5 wt %. In embodiments, the citrus bioflavonoid is hesperidin.

Another aspect of the invention is directed to a topical composition for treating aged skin, the composition including a) a mixture of barrier lipids selected from the group consisting of cholesterol, at least one ceramide, at least one free fatty acid, and mixtures of two or more thereof, in a total of 1 to 50 wt %, preferably 1 to 20 wt %, more preferably 2 to 10 wt %; b) 0.1 to 10 wt % of a skin conditioner, preferably 1 to 5 wt %, more preferably 2 to 4 wt %; c) 0.001 to 5 wt % of a chelating agent, preferably 0.01 to 2 wt %, more preferably 0.1 to 1 wt %; d) 1 to 30 wt % of a humectant, preferably 3 to 20 wt %, more preferably 5 to 10 wt %; e) 0.5 to 20 wt % of an emulsifier, preferably 0.5 to 10 wt %, more preferably 1 to 5 wt %; f) one or more emollients in a total of 1 to 30 wt %, preferably 2 to 20 wt %, more preferably 4 to 10 wt %; g) one or more preservatives in a total of 0.1 to 10 wt %, preferably 0.5 to 5 wt %, more preferably 0.8 to 1.5 wt %; h) 0.01 to 5 wt % of an acid buffer, preferably 0.25 to 3 wt %, more preferably 0.5 to 2 wt %; i) one or more encapsulation aids in a total of 1 to 25 wt %, preferably 5 to 15 wt %, more preferably 8 to 10 wt %; j) 0.01 to 3 wt % of an aqueous phase thickener, preferably 0.1 to 2 wt %, more preferably 0.2 to 1 wt %; and k) 0.001 to 50 wt % of citrus bioflavonoid (e.g., hesperidin, or an analog, pharmaceutically acceptable salt, or prodrug thereof), preferably 1 to 10 wt %, more preferably 2 to 5 wt %. In embodiments, the citrus bioflavonoid is hesperidin.

In embodiments, citrus bioflavonoid (e.g., hesperidin, or an analog, pharmaceutically acceptable salt, or prodrug thereof) is present in about 2 wt %. In embodiments, citrus bioflavonoid (e.g., hesperidin, or an analog, pharmaceutically acceptable salt, or prodrug thereof) is present in about 5 wt %. In embodiments citrus bioflavonoid (e.g., hesperidin, or an analog, pharmaceutically acceptable salt, or prodrug thereof) is present in about 10 wt %. In embodiments, citrus bioflavonoid (e.g., hesperidin, or an analog, pharmaceutically acceptable salt, or prodrug thereof) is present in about 15 wt %. In embodiments, citrus bioflavonoid (e.g., hesperidin, or an analog, pharmaceutically acceptable salt, or prodrug thereof) is present in about 20 wt %. In embodiments, citrus bioflavonoid (e.g., hesperidin, or an analog, pharmaceutically acceptable salt, or prodrug thereof) is present in about 25 wt %. In embodiments, citrus bioflavonoid (e.g., hesperidin, or an analog, pharmaceutically acceptable salt, or prodrug thereof) is present in about 30 wt %. In embodiments, citrus bioflavonoid (e.g., hesperidin, or an analog, pharmaceutically acceptable salt, or prodrug thereof) is present in about 35 wt %.

In embodiments, citrus bioflavonoid (e.g., hesperidin, or an analog, pharmaceutically acceptable salt, or prodrug thereof) is present in 2 wt %. In embodiments, citrus bioflavonoid (e.g., hesperidin, or an analog, pharmaceutically acceptable salt, or prodrug thereof) is present in 5 wt %. In embodiments citrus bioflavonoid (e.g., hesperidin, or an analog, pharmaceutically acceptable salt, or prodrug thereof) is present in 10 wt %. In embodiments, citrus bioflavonoid (e.g., hesperidin, or an analog, pharmaceutically acceptable salt, or prodrug thereof) is present in 15 wt %. In embodiments, citrus bioflavonoid (e.g., hesperidin, or an analog, pharmaceutically acceptable salt, or prodrug thereof) is present in 20 wt %. In embodiments, citrus bioflavonoid (e.g., hesperidin, or an analog, pharmaceutically acceptable salt, or prodrug thereof) is present in 25 wt %. In embodiments, citrus bioflavonoid (e.g., hesperidin, or an analog, pharmaceutically acceptable salt, or prodrug thereof) is present in 30 wt %. In embodiments, citrus bioflavonoid (e.g., hesperidin, or an analog, pharmaceutically acceptable salt, or prodrug thereof) is present in 35 wt %. In embodiments, the citrus bioflavonoid (e.g., hesperidin, or an analog, pharmaceutically acceptable salt, or prodrug thereof) can be contained within its natural source, such as orange rind or peppermint oil, in an amount sufficient to provide the required wt % of the active citrus bioflavonoid (e.g., hesperidin, or an analog, pharmaceutically acceptable salt, or prodrug thereof).

In embodiments, the composition includes a) a mixture of barrier lipids selected from the group consisting of cholesterol, at least one ceramide, at least one free fatty acid, and mixtures of two or more thereof, in a total of about 1 to about 50 wt %, preferably about 1 to about 20 wt %, more preferably about 2 to about 10 wt %; b) about 0.1 to about 10 wt % of a skin conditioner, preferably about 1 to about 5 wt %, more preferably about 2 to about 4 wt %; c) about 0.001 to about 5 wt % of a chelating agent, preferably about 0.01 to about 2 wt %, more preferably about 0.1 to about 1 wt %; d) about 1 to about 30 wt % of a humectant, preferably about 3 to about 20 wt %, more preferably about 5 to about 10 wt %; e) about 0.5 to about 20 wt % of an emulsifier, preferably about 0.5 to about 10 wt %, more preferably about 1 to about 5 wt %; f) one or more emollients in a total of about 1 to about 30 wt %, preferably about 2 to about 20 wt %, more preferably about 4 to about 10 wt %; g) one or more preservatives in a total of about 0.1 to about 10 wt %, preferably about 0.5 to about 5 wt %, more preferably about 0.8 to about 1.5 wt %; h) about 0.01 to about 5 wt % of an acid buffer, preferably about 0.25 to about 3 wt %, more preferably about 0.5 to about 2 wt %; i) one or more encapsulation aids in a total of about 1 to about 25 wt %, preferably about 5 to about 15 wt %, more preferably about 8 to about 10 wt %; j) about 0.01 to about 3 wt % of an aqueous phase thickener, preferably about 0.1 to about 2 wt %, more preferably about 0.2 to about 1 wt %; and k) about 0.001 to about 50 wt % hesperidin, preferably about 1 to about 10 wt %, more preferably about 2 to about 5 wt %.

In embodiments, the composition includes a) a mixture of barrier lipids selected from the group consisting of cholesterol, at least one ceramide, at least one free fatty acid, and mixtures of two or more thereof, in a total of 1 to 50 wt %, preferably 1 to 20 wt %, more preferably 2 to 10 wt %; b) 0.1 to 10 wt % of a skin conditioner, preferably 1 to 5 wt %, more preferably 2 to 4 wt %; c) 0.001 to 5 wt % of a chelating agent, preferably 0.01 to 2 wt %, more preferably 0.1 to 1 wt %; d) 1 to 30 wt % of a humectant, preferably 3 to 20 wt %, more preferably 5 to 10 wt %; e) 0.5 to 20 wt % of an emulsifier, preferably 0.5 to 10 wt %, more preferably 1 to 5 wt %; f) one or more emollients in a total of 1 to 30 wt %, preferably 2 to 20 wt %, more preferably 4 to 10 wt %; g) one or more preservatives in a total of 0.1 to 10 wt %, preferably 0.5 to 5 wt %, more preferably 0.8 to 1.5 wt %; h) 0.01 to 5 wt % of an acid buffer, preferably 0.25 to 3 wt %, more preferably 0.5 to 2 wt %; i) one or more encapsulation aids in a total of 1 to 25 wt %, preferably 5 to 15 wt %, more preferably 8 to 10 wt %; j) 0.01 to 3 wt % of an aqueous phase thickener, preferably 0.1 to 2 wt %, more preferably 0.2 to 1 wt %; and k) 0.001 to 50 wt % hesperidin, preferably 1 to 10 wt %, more preferably 2 to 5 wt %.

In embodiments, the composition includes a) a mixture of barrier lipids selected from the group consisting of cholesterol, at least one ceramide, at least one free fatty acid, and mixtures of two or more thereof, in a total of 1 to 50 wt %, preferably 1 to 20 wt %, more preferably 2 to 10 wt %; b) 0.1 to 10 wt % of a skin conditioner, preferably 1 to 5 wt %, more preferably 2 to 4 wt %; c) 0.001 to 5 wt % of a chelating agent, preferably 0.01 to 2 wt %, more preferably 0.1 to 1 wt %; d) 1 to 30 wt % of a humectant, preferably 3 to 20 wt %, more preferably 5 to 10 wt %; e) 0.5 to 20 wt % of an emulsifier, preferably 0.5 to 10 wt %, more preferably 1 to 5 wt %; f) one or more emollients in a total of 1 to 30 wt %, preferably 2 to 20 wt %, more preferably 4 to 10 wt %; g) one or more preservatives in a total of 0.1 to 10 wt %, preferably 0.5 to 5 wt %, more preferably 0.8 to 1.5 wt %; h) 0.01 to 5 wt % of an acid buffer, preferably 0.25 to 3 wt %, more preferably 0.5 to 2 wt %; i) one or more encapsulation aids in a total of 1 to 25 wt %, preferably 5 to 15 wt %, more preferably 8 to 10 wt %; j) 0.01 to 3 wt % of an aqueous phase thickener, preferably 0.1 to 2 wt %, more preferably 0.2 to 1 wt %; and k) 0.001 to 50 wt % hesperidin, preferably 1 to 10 wt %, more preferably 2 to 5 wt %.

In embodiments, hesperidin is present in about 2 wt %. In embodiments, hesperidin is present in about 5 wt %. In embodiments hesperidin is present in about 10 wt %. In embodiments, hesperidin is present in about 15 wt %. In embodiments, hesperidin is present in about 20 wt %. In embodiments, hesperidin is present in about 25 wt %. In embodiments, hesperidin is present in about 30 wt %. In embodiments, hesperidin is present in about 35 wt %. In embodiments, hesperidin is present in 2 wt %. In embodiments, hesperidin is present in 5 wt %. In embodiments hesperidin is present in 10 wt %. In embodiments, hesperidin is present in 15 wt %. In embodiments, hesperidin is present in 20 wt %. In embodiments, hesperidin is present in 25 wt %. In embodiments, hesperidin is present in 30 wt %. In embodiments, hesperidin is present in 35 wt %. In embodiments, the hesperidin can be contained within its natural source, such as orange rind or peppermint oil, in an amount sufficient to provide the required wt % of the active hesperidin.

Another aspect of the invention is directed to a topical composition for treating aged skin, the composition including a) a mixture of barrier lipids selected from the group consisting of cholesterol, at least one ceramide, at least one free fatty acid, and mixtures of two or more thereof, in a total of 1 to 50 wt %, preferably about 1 to about 20 wt %, more preferably 2 to 10 wt %; b) 0.1 to 10 wt % of a skin conditioner, preferably 1 to 5 wt %, more preferably 2 to 4 wt %; c) 0.001 to 5 wt % of a chelating agent, preferably 0.01 to 2 wt %, more preferably 0.1 to 1 wt %; d) 1 to 30 wt % of a humectant, preferably 3 to 20 wt %, more preferably 5 to 10 wt %; e) 0.5 to 20 wt % of an emulsifier, preferably 0.5 to 10 wt %, more preferably 1 to 5 wt %; f) one or more emollients in a total of 1 to 30 wt %, preferably 2 to 20 wt %, more preferably 4 to 10 wt %; g) one or more preservatives in a total of 0.1 to 10 wt %, preferably 0.5 to 5 wt %, more preferably 0.8 to 1.5 wt %; h) 0.01 to 5 wt % of an acid buffer, preferably 0.25 to 3 wt %, more preferably 0.5 to 2 wt %; i) one or more encapsulation aids in a total of 1 to 25 wt %, preferably 5 to 15 wt %, more preferably 8 to 10 wt %; j) 0.01 to 3 wt % of an aqueous phase thickener, preferably 0.1 to 2 wt %, more preferably 0.2 to 1 wt %; and k) 0.001 to 50 wt % of hesperidin, preferably 1 to 10 wt %, more preferably 2 to 5 wt %. In embodiments, hesperidin is present in 2 wt %. In embodiments, hesperidin is present in 5 wt %. In embodiments, hesperidin is present in 10 wt %. In embodiments, hesperidin is present in 15 wt %. In embodiments, hesperidin is present in 20 wt %. In embodiments, hesperidin is present in 25 wt %. In embodiments, hesperidin is present in 30 wt %. In embodiments, hesperidin is present in 35 wt %.

In embodiments, the mixture of barrier lipids is present in about 2 to about 10 total wt %. In embodiments, the mixture of barrier lipids is present in about 1.0, 1.1, 1.2, 1.3, 1.4, 1.5, 1.6, 1.7, 1.8, 1.9, 2.0, 2.02, 2.04, 2.06, 2.08, 2.1, 2.12, 2.14, 2.16, 2.18, 2.2, 2.22, 2.24, 2.26, 2.28, 2.3, 2.32, 2.34, 2.36, 2.38, 2.4, 2.42, 2.44, 2.46, 2.48, 2.5, 2.52, 2.54, 2.56, 2.58, 2.6, 2.62, 2.64, 2.66, 2.68, 2.7, 2.72, 2.74, 2.76, 2.78, 2.8, 2.82, 2.84, 2.86, 2.88, 2.9, 2.92, 2.94, 2.96, 2.98, 3.0, 3.02, 3.04, 3.06, 3.08, 3.1, 3.12, 3.14, 3.16, 3.18, 3.2, 3.22, 3.24, 3.26, 3.28, 3.3, 3.32, 3.34, 3.36, 3.38, 3.4, 3.42, 3.44, 3.46, 3.48, 3.5, 3.52, 3.54, 3.56, 3.58, 3.6, 3.62, 3.64, 3.66, 3.68, 3.7, 3.72, 3.74, 3.76, 3.78, 3.8, 3.82, 3.84, 3.86, 3.88, 3.9, 3.92, 3.94, 3.96, 3.98, 4.0, 4.02, 4.04, 4.06, 4.08, 4.1, 4.12, 4.14, 4.16, 4.18, 4.2, 4.22, 4.24, 4.26, 4.28, 4.3, 4.32, 4.34, 4.36, 4.38, 4.4, 4.42, 4.44, 4.46, 4.48, 4.5, 4.52, 4.54, 4.56, 4.58, 4.6, 4.62, 4.64, 4.66, 4.68, 4.7, 4.72, 4.74, 4.76, 4.78, 4.8, 4.82, 4.84, 4.86, 4.88, 4.9, 4.92, 4.94, 4.96, 4.98, 5.0, 5.02, 5.04, 5.06, 5.08, 5.1, 5.12, 5.14, 5.16, 5.18, 5.2, 5.22, 5.24, 5.26, 5.28, 5.3, 5.32, 5.34, 5.36, 5.38, 5.4, 5.42, 5.44, 5.46, 5.48, 5.5, 5.52, 5.54, 5.56, 5.58, 5.6, 5.62, 5.64, 5.66, 5.68, 5.7, 5.72, 5.74, 5.76, 5.78, 5.8, 5.82, 5.84, 5.86, 5.88, 5.9, 5.92, 5.94, 5.96, 5.98, 6.0, 6.02, 6.04, 6.06, 6.08, 6.1, 6.12, 6.14, 6.16, 6.18, 6.2, 6.22, 6.24, 6.26, 6.28, 6.3, 6.32, 6.34, 6.36, 6.38, 6.4, 6.42, 6.44, 6.46, 6.48, 6.5, 6.52, 6.54, 6.56, 6.58, 6.6, 6.62, 6.64, 6.66, 6.68, 6.7, 6.72, 6.74, 6.76, 6.78, 6.8, 6.82, 6.84, 6.86, 6.88, 6.9, 6.92, 6.94, 6.96, 6.98, 7.0, 7.02, 7.04, 7.06, 7.08, 7.1, 7.12, 7.14, 7.16, 7.18, 7.2, 7.22, 7.24, 7.26, 7.28, 7.3, 7.32, 7.34, 7.36, 7.38, 7.4, 7.42, 7.44, 7.46, 7.48, 7.5, 7.52, 7.54, 7.56, 7.58, 7.6, 7.62, 7.64, 7.66, 7.68, 7.7, 7.72, 7.74, 7.76, 7.78, 7.8, 7.82, 7.84, 7.86, 7.88, 7.9, 7.92, 7.94, 7.96, 7.98, 8.0, 8.02, 8.04, 8.06, 8.08, 8.1, 8.12, 8.14, 8.16, 8.18, 8.2, 8.22, 8.24, 8.26, 8.28, 8.3, 8.32, 8.34, 8.36, 8.38, 8.4, 8.42, 8.44, 8.46, 8.48, 8.5, 8.52, 8.54, 8.56, 8.58, 8.6, 8.62, 8.64, 8.66, 8.68, 8.7, 8.72, 8.74, 8.76, 8.78, 8.8, 8.82, 8.84, 8.86, 8.88, 8.9, 8.92, 8.94, 8.96, 8.98, 9.0, 9.02, 9.04, 9.06, 9.08, 9.1, 9.12, 9.14, 9.16, 9.18, 9.2, 9.22, 9.24, 9.26, 9.28, 9.3, 9.32, 9.34, 9.36, 9.38, 9.4, 9.42, 9.44, 9.46, 9.48, 9.5, 9.52, 9.54, 9.56, 9.58, 9.6, 9.62, 9.64, 9.66, 9.68, 9.7, 9.72, 9.74, 9.76, 9.78, 9.8, 9.82, 9.84, 9.86, 9.88, 9.9, 9.92, 9.94, 9.96, 9.98, or about 10 total wt %. In embodiments, the mixture of barrier lipids is present in about 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0, 25.0, 26.0, 27.0, 28.0, 29.0, 30.0, 31.0, 32.0, 33.0, 34.0, 35.0, 36.0, 37.0, 38.0, 39.0, 40.0, 41.0, 42.0, 43.0, 44.0, 45.0, 46.0, 47.0, 48.0, 49.0, or about 50.0 wt %. In embodiments, the mixture of barrier lipids is present in about 0.5 to about 10 total wt %. In embodiments, the mixture of barrier lipids is present in about 0.5 to about 5 total wt %. In embodiments, the mixture of barrier lipids is present in about 1 to about 5 total wt %. In embodiments, the mixture of barrier lipids is present in about 2 to about 5 total wt %.

In embodiments, the mixture of barrier lipids is present in about 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or about 1.0 wt %. In embodiments, the mixture of barrier lipids is present in about 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2.0, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3.0, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, 3.95, 4.0, 4.05, 4.1, 4.15, 4.2, 4.25, 4.3, 4.35, 4.4, 4.45, 4.5, 4.55, 4.6, 4.65, 4.7, 4.75, 4.8, 4.85, 4.9, 4.95, or about 5.0 wt %.

In embodiments, the skin conditioner is present in about 0.1 to about 10 wt %. In embodiments, the skin conditioner is present in about 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2.0, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3, 2.31, 2.32, 2.33, 2.34, 2.35, 2.36, 2.37, 2.38, 2.39, 2.4, 2.41, 2.42, 2.43, 2.44, 2.45, 2.46, 2.47, 2.48, 2.49, 2.5, 2.51, 2.52, 2.53, 2.54, 2.55, 2.56, 2.57, 2.58, 2.59, 2.6, 2.61, 2.62, 2.63, 2.64, 2.65, 2.66, 2.67, 2.68, 2.69, 2.7, 2.71, 2.72, 2.73, 2.74, 2.75, 2.76, 2.77, 2.78, 2.79, 2.8, 2.81, 2.82, 2.83, 2.84, 2.85, 2.86, 2.87, 2.88, 2.89, 2.9, 2.91, 2.92, 2.93, 2.94, 2.95, 2.96, 2.97, 2.98, 2.99, or about 3.0 wt %. In embodiments, the skin conditioner is present in about 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or about 10.0 wt %. In embodiments, the skin conditioner is present in about 0.5 to about 8 wt %. In embodiments, the skin conditioner is present in about 0.5 to about 5 wt %. In embodiments, the skin conditioner is present in about 0.5 to about 3 wt %. In embodiments, the skin conditioner is present in about 0.5 to about 2 wt %.

In embodiments, the chelating agent is present in about 0.0001 to about 5.0 wt %. In embodiments, the chelating agent is present in about 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or about 1.0 wt %. In embodiments, the chelating agent is present in about 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2.0, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3.0, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, 3.95, 4.0, 4.05, 4.1, 4.15, 4.2, 4.25, 4.3, 4.35, 4.4, 4.45, 4.5, 4.55, 4.6, 4.65, 4.7, 4.75, 4.8, 4.85, 4.9, 4.95, or about 5.0 wt %. In embodiments, the chelating agent is present in about 0.01 to about 5.0 wt %. In embodiments, the chelating agent is present in about 0.05 to about 3.0 wt %. In embodiments, the chelating agent is present in about 0.05 to about 2.0 wt %. In embodiments, the chelating agent is present in about 0.05 to about 1.0 wt %. In embodiments, the chelating agent is present in about 0.05 to about 0.25 wt %.

In embodiments, the humectant is present in about 1 to about 30 wt %. In embodiments, the humectant is present in about 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0, 25.0, 26.0, 27.0, 28.0, 29.0 or about 30.0 wt %. In embodiments, the humectant is present in about 5 to about 15 wt %. In embodiments, the humectant is present in about 8 to about 13 wt %. In embodiments, the humectant is present in about 9 to about 11 wt %.

In embodiments, the emulsifier is present in about 0.5 to about 20 wt %. In embodiments, the emulsifier is present in about 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2.0, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3, 2.31, 2.32, 2.33, 2.34, 2.35, 2.36, 2.37, 2.38, 2.39, 2.4, 2.41, 2.42, 2.43, 2.44, 2.45, 2.46, 2.47, 2.48, 2.49, 2.5, 2.51, 2.52, 2.53, 2.54, 2.55, 2.56, 2.57, 2.58, 2.59, 2.6, 2.61, 2.62, 2.63, 2.64, 2.65, 2.66, 2.67, 2.68, 2.69, 2.7, 2.71, 2.72, 2.73, 2.74, 2.75, 2.76, 2.77, 2.78, 2.79, 2.8, 2.81, 2.82, 2.83, 2.84, 2.85, 2.86, 2.87, 2.88, 2.89, 2.9, 2.91, 2.92, 2.93, 2.94, 2.95, 2.96, 2.97, 2.98, 2.99, or about 3.0 wt %. In embodiments, the emulsifier is present in about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, or about 20.0 wt %. In embodiments, the emulsifier is present in about 0.5 to about 10 wt %. In embodiments, the emulsifier is present in about 0.5 to about 5 wt %. In embodiments, the emulsifier is present in about 0.5 to about 2 wt %.

In embodiments, one or more emollients is present in about 1 to about 30 total wt %. In embodiments, one or more emollients is present in about 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24.0, 24.5, 25.0, 25.5, 26.0, 26.5, 27.0, 27.5, 28.0, 28.5, 29.0, 29.5, or about 30.0 wt %. In embodiments, one or more emollients is present in about 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2.0, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3, 2.31, 2.32, 2.33, 2.34, 2.35, 2.36, 2.37, 2.38, 2.39, 2.4, 2.41, 2.42, 2.43, 2.44, 2.45, 2.46, 2.47, 2.48, 2.49, or about 2.5 total wt %. In embodiments, one or more emollients is present in about 3.5, 3.55, 3.6, 3.65, 3.7, 3.75, 3.8, 3.85, 3.9, 3.95, 4.0, 4.05, 4.1, 4.15, 4.2, 4.25, 4.3, 4.35, 4.4, 4.45, 4.5, 4.55, 4.6, 4.65, 4.7, 4.75, 4.8, 4.85, 4.9, 4.95, or about 5.0 total wt %. In embodiments, one or more emollients is present in about 1 to about 10 wt %. In embodiments, one or more emollients is present in about 1 to about 5 wt %. In embodiments, one or more emollients is present in about 1 to about 3 wt %.

In embodiments, one or more preservatives is present in about 0.1 to about 10 total wt %. In embodiments, one or more preservatives is present in about 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, or about 1.5 wt %. In embodiments, one or more preservatives is present in about 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or about 1.0. In embodiments, one or more preservatives is present in about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or about 10.0 total wt %. In embodiments, one or more preservatives is present in about 0.1 to about 3 wt %. In embodiments, one or more preservatives is present in about 0.1 to about 2 wt %. In embodiments, one or more preservatives is present in about 0.1 to about 1.0 wt %.

In embodiments, one or more encapsulation aids is present in about 1 to about 25 total wt %. In embodiments, one or more encapsulation aids is present in about 7.0, 7.02, 7.04, 7.06, 7.08, 7.1, 7.12, 7.14, 7.16, 7.18, 7.2, 7.22, 7.24, 7.26, 7.28, 7.3, 7.32, 7.34, 7.36, 7.38, 7.4, 7.42, 7.44, 7.46, 7.48, 7.5, 7.52, 7.54, 7.56, 7.58, 7.6, 7.62, 7.64, 7.66, 7.68, 7.7, 7.72, 7.74, 7.76, 7.78, 7.8, 7.82, 7.84, 7.86, 7.88, 7.9, 7.92, 7.94, 7.96, 7.98, 8.0, 8.02, 8.04, 8.06, 8.08, 8.1, 8.12, 8.14, 8.16, 8.18, 8.2, 8.22, 8.24, 8.26, 8.28, 8.3, 8.32, 8.34, 8.36, 8.38, 8.4, 8.42, 8.44, 8.46, 8.48, 8.5, 8.52, 8.54, 8.56, 8.58, 8.6, 8.62, 8.64, 8.66, 8.68, 8.7, 8.72, 8.74, 8.76, 8.78, 8.8, 8.82, 8.84, 8.86, 8.88, 8.9, 8.92, 8.94, 8.96, 8.98, 9.0, 9.02, 9.04, 9.06, 9.08, 9.1, 9.12, 9.14, 9.16, 9.18, 9.2, 9.22, 9.24, 9.26, 9.28, 9.3, 9.32, 9.34, 9.36, 9.38, 9.4, 9.42, 9.44, 9.46, 9.48, 9.5, 9.52, 9.54, 9.56, 9.58, 9.6, 9.62, 9.64, 9.66, 9.68, 9.7, 9.72, 9.74, 9.76, 9.78, 9.8, 9.82, 9.84, 9.86, 9.88, 9.9, 9.92, 9.94, 9.96, 9.98, or about 10.0 total wt %. In embodiments, one or more encapsulation aids is present in about 6.5, 6.52, 6.54, 6.56, 6.58, 6.6, 6.62, 6.64, 6.66, 6.68, 6.7, 6.72, 6.74, 6.76, 6.78, 6.8, 6.82, 6.84, 6.86, 6.88, 6.9, 6.92, 6.94, 6.96, 6.98, 7.0, 7.02, 7.04, 7.06, 7.08, 7.1, 7.12, 7.14, 7.16, 7.18, 7.2, 7.22, 7.24, 7.26, 7.28, 7.3, 7.32, 7.34, 7.36, 7.38, 7.4, 7.42, 7.44, 7.46, 7.48, or about 7.5 wt %. In embodiments, one or more encapsulation aids is present in about 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or about 1.0 wt %. In embodiments, one or more encapsulation aids is present in about 1 to about 15 total wt %. In embodiments, one or more encapsulation aids is present in about 1 to about 10 total wt %. In embodiments, one or more encapsulation aids is present in about 2 to about 9 total wt %.

In embodiments, aqueous phase thickener is present in about 0.01 to about 3 wt %. In embodiments, aqueous phase thickener is present in about 0.1, 0.12, 0.14, 0.16, 0.18, 0.2, 0.22, 0.24, 0.26, 0.28, 0.3, 0.32, 0.34, 0.36, 0.38, 0.4, 0.42, 0.44, 0.46, 0.48, 0.5, 0.52, 0.54, 0.56, 0.58, 0.6, 0.62, 0.64, 0.66, 0.68, 0.7, 0.72, 0.74, 0.76, 0.78, 0.8, 0.82, 0.84, 0.86, 0.88, 0.9, 0.92, 0.94, 0.96, 0.98, 1.0, 1.02, 1.04, 1.06, 1.08, 1.1, 1.12, 1.14, 1.16, 1.18, 1.2, 1.22, 1.24, 1.26, 1.28, 1.3, 1.32, 1.34, 1.36, 1.38, 1.4, 1.42, 1.44, 1.46, 1.48, 1.5, 1.52, 1.54, 1.56, 1.58, 1.6, 1.62, 1.64, 1.66, 1.68, 1.7, 1.72, 1.74, 1.76, 1.78, 1.8, 1.82, 1.84, 1.86, 1.88, 1.9, 1.92, 1.94, 1.96, 1.98, or about 2.0 wt %. In embodiments, aqueous phase thickener is present in about 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, to about 0.75 wt %. In embodiments, aqueous phase thickener is present in about 0.05 to about 3 wt %. In embodiments, aqueous phase thickener is present in about 0.10 to about 2 wt %. In embodiments, aqueous phase thickener is present in about 0.5 to about 1.0 wt %.

In embodiments, the mixture of barrier lipids consists of cholesterol, hydroxypropyl bispalmitamide MEA (Ceramide PC-104) and conjugated linoleic acid (CLA). In embodiments, the mixture of barrier lipids consists of cholesterol, myristoyl/palmitoyl oxostearamide/arachamide MEA and conjugated linoleic acid (CLA). In embodiments, the mixture of barrier lipids consists of cholesterol, myristoyl/palmitoyl oxostearamide/arachamide and conjugated linoleic acid (CLA). In embodiments, the mixture of barrier lipids consists of cholesterol, hydroxypropyl bispalmitamide MEA (Ceramide PC-104) and conjugated linoleic acid (CLA) present in a 3:1:1 molar ratio. In embodiments, the mixture of barrier lipids consists of cholesterol myristoyl/palmitoyl oxostearamide/arachamide MEA and conjugated linoleic acid (CLA) present in a 3:1:1 molar ratio. In embodiments, the mixture of barrier lipids is cholesterol-dominant. In embodiments, the skin conditioner comprises dimethicone, the chelating agent comprises disodium EDTA, the humectant comprises glycerin, and the acid buffer comprises citric acid. In embodiments, the emulsifier comprises glyceryl stearate PEG-100. In embodiments, the one or more emollients comprise petrolatum and squalane. In embodiments, the one or more preservatives comprise phenoxyethanol and sorbic acid. In embodiments, the one or more encapsulation aids comprise corn syrup solids and euphorbia cerifera (candelilla) wax. In embodiments, the aqueous phase thickener comprises xanthan gum.

In embodiments, the mixture of barrier lipids includes cholesterol, hydroxypropyl bispalmitamide MEA (Ceramide PC-104) and conjugated linoleic acid (CLA). In embodiments, the mixture of barrier lipids includes cholesterol, myristoyl/palmitoyl oxostearamide/arachamide MEA, and conjugated linoleic acid (CLA). In embodiments, the mixture of barrier lipids includes cholesterol, myristoyl/palmitoyl oxostearamide/arachamide, and conjugated linoleic acid (CLA). In embodiments, the mixture of barrier lipids includes cholesterol, hydroxypropyl bispalmitamide MEA (Ceramide PC-104) and conjugated linoleic acid (CLA) present in a 3:1:1 molar ratio. In embodiments, the mixture of barrier lipids is cholesterol-dominant. In embodiments, the skin conditioner is dimethicone, the chelating agent is disodium EDTA, the humectant is glycerin, and the acid buffer is citric acid. In embodiments, the emulsifier is glyceryl stearate PEG-100. In embodiments, the one or more emollients is petrolatum and squalane. In embodiments, the one or more preservatives is phenoxyethanol and sorbic acid. In embodiments, the one or more encapsulation aids is corn syrup solids and euphorbia cerifera (candelilla) wax. In embodiments, the aqueous phase thickener is xanthan gum.

In embodiments, the mixture of barrier lipids is as described herein (e.g., the mixture of barrier lipids as described herein, including in examples). In embodiments, the skin conditioner is as described herein (e.g., the skin conditioner as described herein, including in examples), the chelating agent is as described herein (e.g., the chelating agent as described herein, including in examples), the humectant is as described herein (e.g., the humectant as described herein, including in examples), and the acid buffer is as described herein (e.g., the acid buffer as described herein, including in examples). In embodiments, the emulsifier is as described herein (e.g., the emulsifier as described herein, including in examples). In embodiments, the one or more emollients is as described herein (e.g., the emollients as described herein, including in examples). In embodiments, the one or more preservatives is as described herein (e.g., the preservatives as described herein, including in examples). In embodiments, the one or more encapsulation aids is as described herein (e.g., the encapsulation aids as described herein, including in examples). In embodiments, the aqueous phase thickener is as described herein (e.g., the aqueous phase thickener as described herein, including in examples).

In a preferred embodiment the pH of the topical composition is from about 3 to about 5.5. In embodiments the pH of the topical composition is about 3. In embodiments the pH of the topical composition is about 3.2. In embodiments the pH of the topical composition is about 3.4. In embodiments the pH of the topical composition is about 3.6. In embodiments the pH of the topical composition is about 3.8. In embodiments the pH of the topical composition is about 4. In embodiments the pH of the topical composition is about 4.2. In embodiments the pH of the topical composition is about 4.4. In embodiments the pH of the topical composition is about 4.6. In embodiments the pH of the topical composition is about 4.8. In embodiments the pH of the topical composition is about 5. In embodiments the pH of the topical composition is about 5.2. In embodiments the pH of the topical composition is about 5.5. In a preferred embodiment the pH of the topical composition is about 3.1 to about 5.0. In a preferred embodiment the pH of the topical composition is about 3.2 to about 4.5. In a preferred embodiment the pH of the topical composition is about 3 to about 4.

In a preferred embodiment the pH of the topical composition is 3 to 5.5. In embodiments the pH of the topical composition is 3. In embodiments the pH of the topical composition is 3.2. In embodiments the pH of the topical composition is 3.4. In embodiments the pH of the topical composition is 3.6. In embodiments the pH of the topical composition is 3.8. In embodiments the pH of the topical composition is 4. In embodiments the pH of the topical composition is 4.2. In embodiments the pH of the topical composition is 4.4. In embodiments the pH of the topical composition is 4.6. In embodiments the pH of the topical composition is 4.8. In embodiments the pH of the topical composition is 5. In embodiments the pH of the topical composition is 5.2. In embodiments the pH of the topical composition is 5.5. In a preferred embodiment the pH of the topical composition is 3.1 to 5.0. In a preferred embodiment the pH of the topical composition is 3.2 to 4.5. In a preferred embodiment the pH of the topical composition is 3 to 4.

In embodiments, the topical composition further comprises a skin conditioner, a chelating agent, a humectant, an emulsifier, one or more emollients, one or more preservatives, an acid buffer, one or more encapsulation aids, and an aqueous phase thickener. In embodiments, the topical composition further comprises a skin conditioner, a chelating agent, a humectant, an emulsifier, one or more emollients, one or more preservatives, an acid buffer, one or more encapsulation aids, or an aqueous phase thickener.

Another aspect of the invention is directed to a topical composition for treating aged skin, the composition including a) a mixture of barrier lipids selected from the group consisting of cholesterol, at least one ceramide, at least one free fatty acid, and mixtures of two or more thereof, in a total of 1 to 50 wt %, preferably 1 to 20 wt %, more preferably 2 to 10 wt %; b) 0.1 to 10 wt % of a skin conditioner, preferably 1 to 5 wt %, more preferably 2 to 4 wt %; c) 0.001 to 5 wt % of a chelating agent, preferably 0.01 to 2 wt %, more preferably 0.1 to 1 wt %; d) 1 to 30 wt % of a humectant, preferably 3 to 20 wt %, more preferably 5 to 10 wt %; e) 0.5 to 20 wt % of an emulsifier, preferably 0.5 to 10 wt %, more preferably 1 to 5 wt %; f) one or more emollients in a total of 1 to 30 wt %, preferably 2 to 20 wt %, more preferably 4 to 10 wt %; g) one or more preservatives in a total of 0.1 to 10 wt %, preferably 0.5 to 5 wt %, more preferably 0.8 to 1.5 wt %; h) 0.01 to 5 wt % of an acid buffer, preferably 0.25 to 3 wt %, more preferably 0.5 to 2 wt %; i) one or more encapsulation aids in a total of 1 to 25 wt %, preferably 5 to 15 wt %, more preferably 8 to 10 wt %; j) 0.01 to 3 wt % of an aqueous phase thickener, preferably 0.1 to 2 wt %, more preferably 0.2 to 1 wt %; and k) 0.001 to 50 wt % of citrus bioflavonoid (e.g., hesperidin, or an analog, pharmaceutically acceptable salt, or prodrug thereof), preferably 1 to 10 wt %, more preferably 2 to 5 wt %. In embodiments, the citrus bioflavonoid is hesperidin.

In embodiments, citrus bioflavonoid (e.g., hesperidin, or an analog, pharmaceutically acceptable salt, or prodrug thereof) is present in 2 wt %. In embodiments, citrus bioflavonoid (e.g., hesperidin, or an analog, pharmaceutically acceptable salt, or prodrug thereof) is present in 5 wt %. In embodiments citrus bioflavonoid (e.g., hesperidin, or an analog, pharmaceutically acceptable salt, or prodrug thereof) is present in 10 wt %. In embodiments, citrus bioflavonoid (e.g., hesperidin, or an analog, pharmaceutically acceptable salt, or prodrug thereof) is present in 15 wt %. In embodiments, citrus bioflavonoid (e.g., hesperidin, or an analog, pharmaceutically acceptable salt, or prodrug thereof) is present in 20 wt %. In embodiments, citrus bioflavonoid (e.g., hesperidin, or an analog, pharmaceutically acceptable salt, or prodrug thereof) is present in 25 wt %. In embodiments, citrus bioflavonoid (e.g., hesperidin, or an analog, pharmaceutically acceptable salt, or prodrug thereof) is present in 30 wt %. In embodiments, citrus bioflavonoid (e.g., hesperidin, or an analog, pharmaceutically acceptable salt, or prodrug thereof) is present in 35 wt %. In embodiments, citrus bioflavonoid (e.g., hesperidin, or an analog, pharmaceutically acceptable salt, or prodrug thereof) is present in 2 wt %. In embodiments, citrus bioflavonoid (e.g., hesperidin, or an analog, pharmaceutically acceptable salt, or prodrug thereof) is present in 5 wt %. In embodiments citrus bioflavonoid (e.g., hesperidin, or an analog, pharmaceutically acceptable salt, or prodrug thereof) is present in 10 wt %. In embodiments, citrus bioflavonoid (e.g., hesperidin, or an analog, pharmaceutically acceptable salt, or prodrug thereof) is present in 15 wt %. In embodiments, citrus bioflavonoid (e.g., hesperidin, or an analog, pharmaceutically acceptable salt, or prodrug thereof) is present in 20 wt %. In embodiments, citrus bioflavonoid (e.g., hesperidin, or an analog, pharmaceutically acceptable salt, or prodrug thereof) is present in 25 wt %. In embodiments, citrus bioflavonoid (e.g., hesperidin, or an analog, pharmaceutically acceptable salt, or prodrug thereof) is present in 30 wt %. In embodiments, citrus bioflavonoid (e.g., hesperidin, or an analog, pharmaceutically acceptable salt, or prodrug thereof) is present in 35 wt %. In embodiments, the citrus bioflavonoid (e.g., hesperidin, or an analog, pharmaceutically acceptable salt, or prodrug thereof) can be contained within its natural source, such as orange rind or peppermint oil, in an amount sufficient to provide the required wt % of the active citrus bioflavonoid (e.g., hesperidin, or an analog, pharmaceutically acceptable salt, or prodrug thereof).

Another aspect of the invention is directed to a topical composition for treating aged skin, the composition including a) a mixture of barrier lipids selected from the group consisting of cholesterol, at least one ceramide, at least one free fatty acid, and mixtures of two or more thereof, in a total of 1 to 50 wt %, preferably 1 to 20 wt %, more preferably 2 to 10 wt %; b) 0.1 to 10 wt % of a skin conditioner, preferably 1 to 5 wt %, more preferably 2 to 4 wt %; c) 0.001 to 5 wt % of a chelating agent, preferably 0.01 to 2 wt %, more preferably 0.1 to 1 wt %; d) 1 to 30 wt % of a humectant, preferably 3 to 20 wt %, more preferably 5 to 10 wt %; e) 0.5 to 20 wt % of an emulsifier, preferably 0.5 to 10 wt %, more preferably 1 to 5 wt %; f) one or more emollients in a total of 1 to 30 wt %, preferably 2 to 20 wt %, more preferably 4 to 10 wt %; g) one or more preservatives in a total of 0.1 to 10 wt %, preferably 0.5 to 5 wt %, more preferably 0.8 to 1.5 wt %; h) 0.01 to 5 wt % of an acid buffer, preferably 0.25 to 3 wt %, more preferably 0.5 to 2 wt %; i) one or more encapsulation aids in a total of 1 to 25 wt %, preferably 5 to 15 wt %, more preferably 8 to 10 wt %; j) 0.01 to 3 wt % of an aqueous phase thickener, preferably 0.1 to 2 wt %, more preferably 0.2 to 1 wt %; and k) 0.001 to 50 wt % of citrus bioflavonoid (e.g., hesperidin, or an analog, pharmaceutically acceptable salt, or prodrug thereof), preferably 1 to 10 wt %, more preferably 2 to 5 wt %. In embodiments, the citrus bioflavonoid is hesperidin.

In embodiments, citrus bioflavonoid (e.g., hesperidin, or an analog, pharmaceutically acceptable salt, or prodrug thereof) is present in 2 wt %. In embodiments, citrus bioflavonoid (e.g., hesperidin, or an analog, pharmaceutically acceptable salt, or prodrug thereof) is present in 5 wt %. In embodiments, citrus bioflavonoid (e.g., hesperidin, or an analog, pharmaceutically acceptable salt, or prodrug thereof) is present in 10 wt %. In embodiments, citrus bioflavonoid (e.g., hesperidin, or an analog, pharmaceutically acceptable salt, or prodrug thereof) is present in 15 wt %. In embodiments, citrus bioflavonoid (e.g., hesperidin, or an analog, pharmaceutically acceptable salt, or prodrug thereof) is present in 20 wt %. In embodiments, citrus bioflavonoid (e.g., hesperidin, or an analog, pharmaceutically acceptable salt, or prodrug thereof) is present in 25 wt %. In embodiments, citrus bioflavonoid (e.g., hesperidin, or an analog, pharmaceutically acceptable salt, or prodrug thereof) is present in 30 wt %. In embodiments, citrus bioflavonoid (e.g., hesperidin, or an analog, pharmaceutically acceptable salt, or prodrug thereof) is present in 35 wt %.

Another aspect of the invention is directed to a topical composition for treating aged skin, the composition including a) a mixture of barrier lipids selected from the group consisting of cholesterol, at least one ceramide, at least one free fatty acid, and mixtures of two or more thereof, in a total of 1 to 50 wt %, preferably 1 to 20 wt %, more preferably 2 to 10 wt %; b) 0.1 to 10 wt % of a skin conditioner, preferably 1 to 5 wt %, more preferably 2 to 4 wt %; c) 0.001 to 5 wt % of a chelating agent, preferably 0.01 to 2 wt %, more preferably 0.1 to 1 wt %; d) 1 to 30 wt % of a humectant, preferably 3 to 20 wt %, more preferably 5 to 10 wt %; e) 0.5 to 20 wt % of an emulsifier, preferably 0.5 to 10 wt %, more preferably 1 to 5 wt %; f) one or more emollients in a total of 1 to 30 wt %, preferably 2 to 20 wt %, more preferably 4 to 10 wt %; g) one or more preservatives in a total of 0.1 to 10 wt %, preferably 0.5 to 5 wt %, more preferably 0.8 to 1.5 wt %; h) 0.01 to 5 wt % of an acid buffer, preferably 0.25 to 3 wt %, more preferably 0.5 to 2 wt %; i) one or more encapsulation aids in a total of 1 to 25 wt %, preferably 5 to 15 wt %, more preferably 8 to 10 wt %; j) 0.01 to 3 wt % of an aqueous phase thickener, preferably 0.1 to 2 wt %, more preferably 0.2 to 1 wt %; and k) 0.001 to 50 wt % of hesperidin or an analog, pharmaceutically acceptable salt, or prodrug thereof, preferably 1 to 10 wt %, more preferably 2 to 5 wt %.

In embodiments, hesperidin or an analog, pharmaceutically acceptable salt, or prodrug thereof is present in 2 wt %. In embodiments, hesperidin or an analog, pharmaceutically acceptable salt, or prodrug thereof is present in 5 wt %. In embodiments, hesperidin or an analog, pharmaceutically acceptable salt, or prodrug thereof is present in 10 wt %. In embodiments, hesperidin or an analog, pharmaceutically acceptable salt, or prodrug thereof is present in 15 wt %. In embodiments, hesperidin or an analog, pharmaceutically acceptable salt, or prodrug thereof is present in 20 wt %. In embodiments, hesperidin or an analog, pharmaceutically acceptable salt, or prodrug thereof is present in 25 wt %. In embodiments, hesperidin or an analog, pharmaceutically acceptable salt, or prodrug thereof is present in 30 wt %. In embodiments, hesperidin or an analog, pharmaceutically acceptable salt, or prodrug thereof is present in 35 wt %. In embodiments, hesperidin or an analog, pharmaceutically acceptable salt, or prodrug thereof is present in 2 wt %. In embodiments, hesperidin or an analog, pharmaceutically acceptable salt, or prodrug thereof is present in 5 wt %. In embodiments hesperidin or an analog, pharmaceutically acceptable salt, or prodrug thereof is present in 10 wt %. In embodiments, hesperidin or an analog, pharmaceutically acceptable salt, or prodrug thereof is present in 15 wt %. In embodiments, hesperidin is present in 20 wt %. In embodiments, hesperidin or an analog, pharmaceutically acceptable salt, or prodrug thereof is present in 25 wt %. In embodiments, hesperidin or an analog, pharmaceutically acceptable salt, or prodrug thereof is present in 30 wt %. In embodiments, hesperidin or an analog, pharmaceutically acceptable salt, or prodrug thereof is present in 35 wt %. In embodiments, the hesperidin or an analog, pharmaceutically acceptable salt, or prodrug thereof can be contained within its natural source, such as orange rind or peppermint oil, in an amount sufficient to provide the required wt % of the active hesperidin or an analog, pharmaceutically acceptable salt, or prodrug thereof.

Another aspect of the invention is directed to a topical composition for treating aged skin, the composition including a) a mixture of barrier lipids selected from the group consisting of cholesterol, at least one ceramide, at least one free fatty acid, and mixtures of two or more thereof, in a total of 1 to 50 wt %, preferably 1 to 20 wt %, more preferably 2 to 10 wt %; b) 0.1 to 10 wt % of a skin conditioner, preferably 1 to 5 wt %, more preferably 2 to 4 wt %; c) 0.001 to 5 wt % of a chelating agent, preferably 0.01 to 2 wt %, more preferably 0.1 to 1 wt %; d) 1 to 30 wt % of a humectant, preferably 3 to 20 wt %, more preferably 5 to 10 wt %; e) 0.5 to 20 wt % of an emulsifier, preferably 0.5 to 10 wt %, more preferably 1 to 5 wt %; f) one or more emollients in a total of 1 to 30 wt %, preferably 2 to 20 wt %, more preferably 4 to 10 wt %; g) one or more preservatives in a total of 0.1 to 10 wt %, preferably 0.5 to 5 wt %, more preferably 0.8 to 1.5 wt %; h) 0.01 to 5 wt % of an acid buffer, preferably 0.25 to 3 wt %, more preferably 0.5 to 2 wt %; i) one or more encapsulation aids in a total of 1 to 25 wt %, preferably 5 to 15 wt %, more preferably 8 to 10 wt %; j) 0.01 to 3 wt % of an aqueous phase thickener, preferably 0.1 to 2 wt %, more preferably 0.2 to 1 wt %; and k) 0.001 to 50 wt % of hesperidin, preferably 1 to 10 wt %, more preferably 2 to 5 wt %.

In embodiments, hesperidin is present in 2 wt %. In embodiments, hesperidin is present in 5 wt %. In embodiments hesperidin is present in 10 wt %. In embodiments, hesperidin is present in 15 wt %. In embodiments, hesperidin is present in 20 wt %. In embodiments, hesperidin is present in 25 wt %. In embodiments, hesperidin is present in 30 wt %. In embodiments, hesperidin is present in 35 wt %. In embodiments, hesperidin is present in 2 wt %. In embodiments, hesperidin is present in 5 wt %. In embodiments hesperidin is present in 10 wt %. In embodiments, hesperidin is present in 15 wt %. In embodiments, hesperidin is present in 20 wt %. In embodiments, hesperidin is present in 25 wt %. In embodiments, hesperidin is present in 30 wt %. In embodiments, hesperidin is present in 35 wt %. In embodiments, the hesperidin can be contained within its natural source, such as orange rind or peppermint oil, in an amount sufficient to provide the required wt % of the active hesperidin.

An aspect of the invention is directed to a topical composition for treating aged skin, the composition including a) a mixture of barrier lipids selected from the group consisting of cholesterol, at least one ceramide, at least one free fatty acid, and mixtures of two or more thereof, in a total of 1 to 50 wt %, preferably 1 to 20 wt %, more preferably 2 to 10 wt %; b) 0.1 to 10 wt % of a skin conditioner, preferably 1 to 5 wt %, more preferably 2 to 4 wt %; c) 0.001 to 5 wt % of a chelating agent, preferably 0.01 to 2 wt %, more preferably 0.1 to 1 wt %; d) 1 to 30 wt % of a humectant, preferably 3 to 20 wt %, more preferably 5 to 10 wt %; e) 0.5 to 20 wt % of an emulsifier, preferably 0.5 to 10 wt %, more preferably 1 to 5 wt %; f) one or more emollients in a total of 1 to 30 wt %, preferably 2 to 20 wt %, more preferably 4 to 10 wt %; g) one or more preservatives in a total of 0.1 to 10 wt %, preferably 0.5 to 5 wt %, more preferably 0.8 to 1.5 wt %; h) 0.01 to 5 wt % of an acid buffer, preferably 0.25 to 3 wt %, more preferably 0.5 to 2 wt %; i) one or more encapsulation aids in a total of 1 to 25 wt %, preferably 5 to 15 wt %, more preferably 8 to 10 wt %; j) 0.01 to 3 wt % of an aqueous phase thickener, preferably 0.1 to 2 wt %, more preferably 0.2 to 1 wt %; and k) 0.001 to 50 wt % of citrus bioflavonoid (e.g., hesperidin, or an analog, pharmaceutically acceptable salt, or prodrug thereof), preferably 1 to 10 wt %, more preferably 2 to 5 wt %. In embodiments, the citrus bioflavonoid is hesperidin.

In embodiments, the composition includes a) a mixture of barrier lipids selected from the group consisting of cholesterol, at least one ceramide, at least one free fatty acid, and mixtures of two or more thereof, in a total of 1 to 50 wt %, preferably 1 to 20 wt %, more preferably 2 to 10 wt %; b) 0.1 to 10 wt % of a skin conditioner, preferably 1 to 5 wt %, more preferably 2 to 4 wt %; c) 0.001 to 5 wt % of a chelating agent, preferably 0.01 to 2 wt %, more preferably 0.1 to 1 wt %; d) 1 to 30 wt % of a humectant, preferably 3 to 20 wt %, more preferably 5 to 10 wt %; e) 0.5 to 20 wt % of an emulsifier, preferably 0.5 to 10 wt %, more preferably 1 to 5 wt %; f) one or more emollients in a total of 1 to 30 wt %, preferably 2 to 20 wt %, more preferably 4 to 10 wt %; g) one or more preservatives in a total of 0.1 to 10 wt %, preferably 0.5 to 5 wt %, more preferably 0.8 to 1.5 wt %; h) 0.01 to 5 wt % of an acid buffer, preferably 0.25 to 3 wt %, more preferably 0.5 to 2 wt %; i) one or more encapsulation aids in a total of 1 to 25 wt %, preferably 5 to 15 wt %, more preferably 8 to 10 wt %; j) 0.01 to 3 wt % of an aqueous phase thickener, preferably 0.1 to 2 wt %, more preferably 0.2 to 1 wt %; and k) 0.001 to 50 wt % of hesperidin or an analog, pharmaceutically acceptable salt, or prodrug thereof, preferably 1 to 10 wt %, more preferably 2 to 5 wt %.

In embodiments, hesperidin or an analog, pharmaceutically acceptable salt, or prodrug thereof is present in 2 wt %. In embodiments, hesperidin or an analog, pharmaceutically acceptable salt, or prodrug thereof is present in 5 wt %. In embodiments, hesperidin or an analog, pharmaceutically acceptable salt, or prodrug thereof is present in 10 wt %. In embodiments, hesperidin or an analog, pharmaceutically acceptable salt, or prodrug thereof is present in 15 wt %. In embodiments, hesperidin or an analog, pharmaceutically acceptable salt, or prodrug thereof is present in 20 wt %. In embodiments, hesperidin or an analog, pharmaceutically acceptable salt, or prodrug thereof is present in 25 wt %. In embodiments, hesperidin or an analog, pharmaceutically acceptable salt, or prodrug thereof is present in 30 wt %. In embodiments, hesperidin or an analog, pharmaceutically acceptable salt, or prodrug thereof is present in 35 wt %.

In embodiments, the composition includes a) a mixture of barrier lipids selected from the group consisting of cholesterol, at least one ceramide, at least one free fatty acid, and mixtures of two or more thereof, in a total of 1 to 50 wt %, preferably 1 to 20 wt %, more preferably 2 to 10 wt %; b) 0.1 to 10 wt % of a skin conditioner, preferably 1 to 5 wt %, more preferably 2 to 4 wt %; c) 0.001 to 5 wt % of a chelating agent, preferably 0.01 to 2 wt %, more preferably 0.1 to 1 wt %; d) 1 to 30 wt % of a humectant, preferably 3 to 20 wt %, more preferably 5 to 10 wt %; e) 0.5 to 20 wt % of an emulsifier, preferably 0.5 to 10 wt %, more preferably 1 to 5 wt %; f) one or more emollients in a total of 1 to 30 wt %, preferably 2 to 20 wt %, more preferably 4 to 10 wt %; g) one or more preservatives in a total of 0.1 to 10 wt %, preferably 0.5 to 5 wt %, more preferably 0.8 to 1.5 wt %; h) 0.01 to 5 wt % of an acid buffer, preferably 0.25 to 3 wt %, more preferably 0.5 to 2 wt %; i) one or more encapsulation aids in a total of 1 to 25 wt %, preferably 5 to 15 wt %, more preferably 8 to 10 wt %; j) 0.01 to 3 wt % of an aqueous phase thickener, preferably 0.1 to 2 wt %, more preferably 0.2 to 1 wt %; and k) 0.001 to 50 wt % of hesperidin, preferably 1 to 10 wt %, more preferably 2 to 5 wt %.

In embodiments, hesperidin is present in 2 wt %. In embodiments, hesperidin is present in 5 wt %. In embodiments, hesperidin is present in 10 wt %. In embodiments, hesperidin is present in 15 wt %. In embodiments, hesperidin is present in 20 wt %. In embodiments, hesperidin is present in 25 wt %. In embodiments, hesperidin is present in 30 wt %. In embodiments, hesperidin is present in 35 wt %.

In embodiments, the mixture of barrier lipids is present in 2 to 10 total wt %. In embodiments, the mixture of barrier lipids is present in 1.0, 1.1, 1.2, 1.3, 1.4, 1.5, 1.6, 1.7, 1.8, 1.9, 2.0, 2.02, 2.04, 2.06, 2.08, 2.1, 2.12, 2.14, 2.16, 2.18, 2.2, 2.22, 2.24, 2.26, 2.28, 2.3, 2.32, 2.34, 2.36, 2.38, 2.4, 2.42, 2.44, 2.46, 2.48, 2.5, 2.52, 2.54, 2.56, 2.58, 2.6, 2.62, 2.64, 2.66, 2.68, 2.7, 2.72, 2.74, 2.76, 2.78, 2.8, 2.82, 2.84, 2.86, 2.88, 2.9, 2.92, 2.94, 2.96, 2.98, 3.0, 3.02, 3.04, 3.06, 3.08, 3.1, 3.12, 3.14, 3.16, 3.18, 3.2, 3.22, 3.24, 3.26, 3.28, 3.3, 3.32, 3.34, 3.36, 3.38, 3.4, 3.42, 3.44, 3.46, 3.48, 3.5, 3.52, 3.54, 3.56, 3.58, 3.6, 3.62, 3.64, 3.66, 3.68, 3.7, 3.72, 3.74, 3.76, 3.78, 3.8, 3.82, 3.84, 3.86, 3.88, 3.9, 3.92, 3.94, 3.96, 3.98, 4.0, 4.02, 4.04, 4.06, 4.08, 4.1, 4.12, 4.14, 4.16, 4.18, 4.2, 4.22, 4.24, 4.26, 4.28, 4.3, 4.32, 4.34, 4.36, 4.38, 4.4, 4.42, 4.44, 4.46, 4.48, 4.5, 4.52, 4.54, 4.56, 4.58, 4.6, 4.62, 4.64, 4.66, 4.68, 4.7, 4.72, 4.74, 4.76, 4.78, 4.8, 4.82, 4.84, 4.86, 4.88, 4.9, 4.92, 4.94, 4.96, 4.98, 5.0, 5.02, 5.04, 5.06, 5.08, 5.1, 5.12, 5.14, 5.16, 5.18, 5.2, 5.22, 5.24, 5.26, 5.28, 5.3, 5.32, 5.34, 5.36, 5.38, 5.4, 5.42, 5.44, 5.46, 5.48, 5.5, 5.52, 5.54, 5.56, 5.58, 5.6, 5.62, 5.64, 5.66, 5.68, 5.7, 5.72, 5.74, 5.76, 5.78, 5.8, 5.82, 5.84, 5.86, 5.88, 5.9, 5.92, 5.94, 5.96, 5.98, 6.0, 6.02, 6.04, 6.06, 6.08, 6.1, 6.12, 6.14, 6.16, 6.18, 6.2, 6.22, 6.24, 6.26, 6.28, 6.3, 6.32, 6.34, 6.36, 6.38, 6.4, 6.42, 6.44, 6.46, 6.48, 6.5, 6.52, 6.54, 6.56, 6.58, 6.6, 6.62, 6.64, 6.66, 6.68, 6.7, 6.72, 6.74, 6.76, 6.78, 6.8, 6.82, 6.84, 6.86, 6.88, 6.9, 6.92, 6.94, 6.96, 6.98, 7.0, 7.02, 7.04, 7.06, 7.08, 7.1, 7.12, 7.14, 7.16, 7.18, 7.2, 7.22, 7.24, 7.26, 7.28, 7.3, 7.32, 7.34, 7.36, 7.38, 7.4, 7.42, 7.44, 7.46, 7.48, 7.5, 7.52, 7.54, 7.56, 7.58, 7.6, 7.62, 7.64, 7.66, 7.68, 7.7, 7.72, 7.74, 7.76, 7.78, 7.8, 7.82, 7.84, 7.86, 7.88, 7.9, 7.92, 7.94, 7.96, 7.98, 8.0, 8.02, 8.04, 8.06, 8.08, 8.1, 8.12, 8.14, 8.16, 8.18, 8.2, 8.22, 8.24, 8.26, 8.28, 8.3, 8.32, 8.34, 8.36, 8.38, 8.4, 8.42, 8.44, 8.46, 8.48, 8.5, 8.52, 8.54, 8.56, 8.58, 8.6, 8.62, 8.64, 8.66, 8.68, 8.7, 8.72, 8.74, 8.76, 8.78, 8.8, 8.82, 8.84, 8.86, 8.88, 8.9, 8.92, 8.94, 8.96, 8.98, 9.0, 9.02, 9.04, 9.06, 9.08, 9.1, 9.12, 9.14, 9.16, 9.18, 9.2, 9.22, 9.24, 9.26, 9.28, 9.3, 9.32, 9.34, 9.36, 9.38, 9.4, 9.42, 9.44, 9.46, 9.48, 9.5, 9.52, 9.54, 9.56, 9.58, 9.6, 9.62, 9.64, 9.66, 9.68, 9.7, 9.72, 9.74, 9.76, 9.78, 9.8, 9.82, 9.84, 9.86, 9.88, 9.9, 9.92, 9.94, 9.96, 9.98, or 10 total wt %. In embodiments, the mixture of barrier lipids is present in 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0, 25.0, 26.0, 27.0, 28.0, 29.0, 30.0, 31.0, 32.0, 33.0, 34.0, 35.0, 36.0, 37.0, 38.0, 39.0, 40.0, 41.0, 42.0, 43.0, 44.0, 45.0, 46.0, 47.0, 48.0, 49.0, or 50.0 wt %.

In embodiments, the mixture of barrier lipids is present in 0.5 to 10 total wt %. In embodiments, the mixture of barrier lipids is present in 0.5 to 5 total wt %. In embodiments, the mixture of barrier lipids is present in 1 to 5 total wt %. In embodiments, the mixture of barrier lipids is present in 2 to 5 total wt %.

In embodiments, the mixture of barrier lipids is present in 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1.0 wt %. In embodiments, the mixture of barrier lipids is present in 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2.0, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3.0, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, 3.95, 4.0, 4.05, 4.1, 4.15, 4.2, 4.25, 4.3, 4.35, 4.4, 4.45, 4.5, 4.55, 4.6, 4.65, 4.7, 4.75, 4.8, 4.85, 4.9, 4.95, or 5.0 wt %.

In embodiments, the skin conditioner is present in 0.1 to 10 wt %. In embodiments, the skin conditioner is present in 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2.0, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3, 2.31, 2.32, 2.33, 2.34, 2.35, 2.36, 2.37, 2.38, 2.39, 2.4, 2.41, 2.42, 2.43, 2.44, 2.45, 2.46, 2.47, 2.48, 2.49, 2.5, 2.51, 2.52, 2.53, 2.54, 2.55, 2.56, 2.57, 2.58, 2.59, 2.6, 2.61, 2.62, 2.63, 2.64, 2.65, 2.66, 2.67, 2.68, 2.69, 2.7, 2.71, 2.72, 2.73, 2.74, 2.75, 2.76, 2.77, 2.78, 2.79, 2.8, 2.81, 2.82, 2.83, 2.84, 2.85, 2.86, 2.87, 2.88, 2.89, 2.9, 2.91, 2.92, 2.93, 2.94, 2.95, 2.96, 2.97, 2.98, 2.99, or 3.0 wt %. In embodiments, the skin conditioner is present in 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10.0 wt %. In embodiments, the skin conditioner is present in 0.5 to 8 wt %. In embodiments, the skin conditioner is present in 0.5 to 5 wt %. In embodiments, the skin conditioner is present in 0.5 to 3 wt %. In embodiments, the skin conditioner is present in 0.5 to 2 wt %.

In embodiments, the chelating agent is present in 0.0001 to 5.0 wt %. In embodiments, the chelating agent is present in 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1.0 wt %. In embodiments, the chelating agent is present in 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2.0, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3.0, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, 3.95, 4.0, 4.05, 4.1, 4.15, 4.2, 4.25, 4.3, 4.35, 4.4, 4.45, 4.5, 4.55, 4.6, 4.65, 4.7, 4.75, 4.8, 4.85, 4.9, 4.95, or 5.0 wt %. In embodiments, the chelating agent is present in 0.01 to 5.0 wt %. In embodiments, the chelating agent is present in 0.05 to 3.0 wt %. In embodiments, the chelating agent is present in 0.05 to 2.0 wt %. In embodiments, the chelating agent is present in 0.01 to 1.0 wt %. In embodiments, the chelating agent is present in 0.05 to 0.25 wt %.

In embodiments, the humectant is present in 1 to 30 wt %. In embodiments, the humectant is present in 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0, 25.0, 26.0, 27.0, 28.0, 29.0 or 30.0 wt %. In embodiments, the humectant is present in 5 to 15 wt %. In embodiments, the humectant is present in 8 to 13 wt %. In embodiments, the humectant is present in 9 to 11 wt %.

In embodiments, the emulsifier is present in 0.5 to 20 wt %. In embodiments, the emulsifier is present in 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2.0, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3, 2.31, 2.32, 2.33, 2.34, 2.35, 2.36, 2.37, 2.38, 2.39, 2.4, 2.41, 2.42, 2.43, 2.44, 2.45, 2.46, 2.47, 2.48, 2.49, 2.5, 2.51, 2.52, 2.53, 2.54, 2.55, 2.56, 2.57, 2.58, 2.59, 2.6, 2.61, 2.62, 2.63, 2.64, 2.65, 2.66, 2.67, 2.68, 2.69, 2.7, 2.71, 2.72, 2.73, 2.74, 2.75, 2.76, 2.77, 2.78, 2.79, 2.8, 2.81, 2.82, 2.83, 2.84, 2.85, 2.86, 2.87, 2.88, 2.89, 2.9, 2.91, 2.92, 2.93, 2.94, 2.95, 2.96, 2.97, 2.98, 2.99, or 3.0 wt %. In embodiments, the emulsifier is present in 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, or 20.0 wt %. In embodiments, the emulsifier is present in 0.5 to 10 wt %. In embodiments, the emulsifier is present in 0.5 to 5 wt %. In embodiments, the emulsifier is present in 0.5 to 2 wt %.

In embodiments, one or more emollients is present in 1 to 30 total wt %. In embodiments, one or more emollients is present in 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24.0, 24.5, 25.0, 25.5, 26.0, 26.5, 27.0, 27.5, 28.0, 28.5, 29.0, 29.5, or 30.0 wt %. In embodiments, one or more emollients is present in 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2.0, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3, 2.31, 2.32, 2.33, 2.34, 2.35, 2.36, 2.37, 2.38, 2.39, 2.4, 2.41, 2.42, 2.43, 2.44, 2.45, 2.46, 2.47, 2.48, 2.49, or 2.5 total wt %. In embodiments, one or more emollients is present in 3.5, 3.55, 3.6, 3.65, 3.7, 3.75, 3.8, 3.85, 3.9, 3.95, 4.0, 4.05, 4.1, 4.15, 4.2, 4.25, 4.3, 4.35, 4.4, 4.45, 4.5, 4.55, 4.6, 4.65, 4.7, 4.75, 4.8, 4.85, 4.9, 4.95, or 5.0 total wt %. In embodiments, one or more emollients is present in 1 to 10 total wt %. In embodiments, one or more emollients is present in 1 to 5 total wt %. In embodiments, one or more emollients is present in 1 to 3 total wt %.

In embodiments, one or more preservatives is present in 0.1 to 10 total wt %. In embodiments, one or more preservatives is present in 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, or 1.5 wt %. In embodiments, one or more preservatives is present in 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1.0. In embodiments, one or more preservatives is present in 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10.0 total wt %. In embodiments, one or more preservatives is present in 0.1 to 3 wt %. In embodiments, one or more preservatives is present in 0.1 to 2 wt %. In embodiments, one or more preservatives is present in 0.1 to 1.0 wt %.

In embodiments, one or more encapsulation aids is present in 1 to 25 total wt %. In embodiments, one or more encapsulation aids is present in 7.0, 7.02, 7.04, 7.06, 7.08, 7.1, 7.12, 7.14, 7.16, 7.18, 7.2, 7.22, 7.24, 7.26, 7.28, 7.3, 7.32, 7.34, 7.36, 7.38, 7.4, 7.42, 7.44, 7.46, 7.48, 7.5, 7.52, 7.54, 7.56, 7.58, 7.6, 7.62, 7.64, 7.66, 7.68, 7.7, 7.72, 7.74, 7.76, 7.78, 7.8, 7.82, 7.84, 7.86, 7.88, 7.9, 7.92, 7.94, 7.96, 7.98, 8.0, 8.02, 8.04, 8.06, 8.08, 8.1, 8.12, 8.14, 8.16, 8.18, 8.2, 8.22, 8.24, 8.26, 8.28, 8.3, 8.32, 8.34, 8.36, 8.38, 8.4, 8.42, 8.44, 8.46, 8.48, 8.5, 8.52, 8.54, 8.56, 8.58, 8.6, 8.62, 8.64, 8.66, 8.68, 8.7, 8.72, 8.74, 8.76, 8.78, 8.8, 8.82, 8.84, 8.86, 8.88, 8.9, 8.92, 8.94, 8.96, 8.98, 9.0, 9.02, 9.04, 9.06, 9.08, 9.1, 9.12, 9.14, 9.16, 9.18, 9.2, 9.22, 9.24, 9.26, 9.28, 9.3, 9.32, 9.34, 9.36, 9.38, 9.4, 9.42, 9.44, 9.46, 9.48, 9.5, 9.52, 9.54, 9.56, 9.58, 9.6, 9.62, 9.64, 9.66, 9.68, 9.7, 9.72, 9.74, 9.76, 9.78, 9.8, 9.82, 9.84, 9.86, 9.88, 9.9, 9.92, 9.94, 9.96, 9.98, or 10.0 total wt %. In embodiments, one or more encapsulation aids is present in 6.5, 6.52, 6.54, 6.56, 6.58, 6.6, 6.62, 6.64, 6.66, 6.68, 6.7, 6.72, 6.74, 6.76, 6.78, 6.8, 6.82, 6.84, 6.86, 6.88, 6.9, 6.92, 6.94, 6.96, 6.98, 7.0, 7.02, 7.04, 7.06, 7.08, 7.1, 7.12, 7.14, 7.16, 7.18, 7.2, 7.22, 7.24, 7.26, 7.28, 7.3, 7.32, 7.34, 7.36, 7.38, 7.4, 7.42, 7.44, 7.46, 7.48, or 7.5 wt %. In embodiments, one or more encapsulation aids is present in 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1.0 wt %. In embodiments, one or more encapsulation aids is present in 1 to 15 total wt %. In embodiments, one or more encapsulation aids is present in 1 to 10 total wt %. In embodiments, one or more encapsulation aids is present in 2 to 9 total wt %.

In embodiments, aqueous phase thickener is present in 0.01 to 3 wt %. In embodiments, aqueous phase thickener is present in 0.1, 0.12, 0.14, 0.16, 0.18, 0.2, 0.22, 0.24, 0.26, 0.28, 0.3, 0.32, 0.34, 0.36, 0.38, 0.4, 0.42, 0.44, 0.46, 0.48, 0.5, 0.52, 0.54, 0.56, 0.58, 0.6, 0.62, 0.64, 0.66, 0.68, 0.69, 0.7, 0.72, 0.74, 0.76, 0.78, 0.8, 0.82, 0.84, 0.86, 0.88, 0.9, 0.92, 0.94, 0.96, 0.98, 1.0, 1.02, 1.04, 1.06, 1.08, 1.1, 1.12, 1.14, 1.16, 1.18, 1.2, 1.22, 1.24, 1.26, 1.28, 1.3, 1.32, 1.34, 1.36, 1.38, 1.4, 1.42, 1.44, 1.46, 1.48, 1.5, 1.52, 1.54, 1.56, 1.58, 1.6, 1.62, 1.64, 1.66, 1.68, 1.7, 1.72, 1.74, 1.76, 1.78, 1.8, 1.82, 1.84, 1.86, 1.88, 1.9, 1.92, 1.94, 1.96, 1.98, or 2.0 wt %. In embodiments, aqueous phase thickener is present in 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, to 0.75 wt %. In embodiments, aqueous phase thickener is present in 0.05 to 3 wt %. In embodiments, aqueous phase thickener is present in 0.10 to 2 wt %. In embodiments, aqueous phase thickener is present in 0.5 to 1.0 wt %.

In a preferred embodiment the molar ratio of cholesterol:ceramide:free fatty acid is 3:1:1. In a preferred embodiment the molar ratio of cholesterol:ceramide:free fatty acid is about 3:about 1:about 1. In a preferred embodiment the molar ratio of cholesterol:ceramide:free fatty acid is 2.9:1:1. In a preferred embodiment the molar ratio of cholesterol:ceramide:free fatty acid is 3:1.1:1. In a preferred embodiment the molar ratio of cholesterol:ceramide:free fatty acid is 3:1:1.1. In a preferred embodiment the molar ratio of cholesterol:ceramide:free fatty acid is 3.1:1:1. In a preferred embodiment the molar ratio of cholesterol:ceramide:free fatty acid is (2.8-3.2):(0.8-1.2):(0.8-1.2). In embodiments, the cholesterol is present in about 0.1 to about 10 wt %, preferably about 1 to about 10 wt %, more preferably about 1 to about 5 wt %. In embodiments, the ceramide or mixture of ceramides is present in about 0.1 to about 10 wt %, preferably about 0.1 to about 5 wt %, more preferably about 0.2 to about 3 wt %. In embodiments, the free fatty acid or mixture of free fatty acids is present in about 0.01 to about 6 wt %, preferably about 0.1 to about 5 wt %, more preferably about 0.2 to about 3 wt %. In embodiments, the cholesterol is present in 0.1 to 10 wt %, preferably 1 to 10 wt %, more preferably 1 to 5 wt %. In embodiments, the ceramide or mixture of ceramides is present in 0.1 to 10 wt %, preferably 0.1 to 5 wt %, more preferably 0.2 to 3 wt %. In embodiments, the free fatty acid or mixture of free fatty acids is present in 0.01 to 6 wt %, preferably 0.1 to 5 wt %, more preferably 0.2 to 3 wt %.

One aspect of the invention is directed to a topical composition, the composition consisting of a) about 1 to about 50 wt % of a mixture of barrier lipids consisting of cholesterol, one or more ceramides, and one or more free fatty acids, preferably about 1 to about 20 wt %, more preferably about 2 to about 10 wt %; b) about 0.1 to about 10 wt % of a skin conditioner, preferably about 1 to about 5 wt %, more preferably about 2 to about 4 wt %; c) about 0.001 to about 5 wt % of a chelating agent, preferably about 0.01 to about 2 wt %, more preferably about 0.1 to about 1 wt %; d) about 1 to about 30 wt % of a humectant, preferably about 3 to about 20 wt %, more preferably about 5 to about 10 wt %; e) about 0.5 to about 20 wt % of glyceryl stearate PEG-100 emulsifier, preferably about 0.5 to about 10 wt %, more preferably about 1 to about 5 wt %; f) about 1 to about 30 wt % of a mixture of emollients consisting of petrolatum and squalane, preferably about 2 to about 20 wt %, more preferably about 4 to about 10 wt %; g) about 0.1 to about 10 wt % of a mixture of preservatives consisting of phenoxyethanol and sorbic acid, preferably about 0.5 to about 5 wt %, more preferably about 0.8 to about 1.5 wt %; h) about 0.01 to about 5 wt % of citric acid, preferably about 0.25 to about 3 wt %, more preferably about 0.5 to about 2 wt %; i) about 1 to about 25 wt % of a mixture of encapsulation aids consisting of corn syrup solids and euphorbia cerifera (candelilla) wax, preferably about 5 to about 15 wt %, more preferably about 8 to about 10 wt %; j) about 0.01 to about 3 wt % of an aqueous phase thickener, preferably about 0.1 to about 2 wt %, more preferably about 0.2 to about 1 wt %; k) about 0.001 to about 50 wt % of hesperidin or an analog, pharmaceutically acceptable salt, or prodrug thereof, preferably about 1 to about 10 wt %, more preferably about 2 to about 5 wt %; and l) water.

In embodiments, hesperidin is present in about 2 wt %. In embodiments, hesperidin is present in about 5 wt %. In embodiments, hesperidin is present in about 10 wt %. In embodiments, hesperidin is present in about 15 wt %. In embodiments, hesperidin is present in about 20 wt %. In embodiments, hesperidin is present in about 25 wt %. In embodiments, hesperidin is present in about 30 wt %. In embodiments, hesperidin is present in about 35 wt %. In embodiments, the hesperidin can be contained within its natural source, such as orange rind or peppermint oil, in an amount sufficient to provide the required wt % of the active hesperidin. The aqueous phase thickener can be, for example, xanthan gum.

One aspect of the invention is directed to a topical composition, the composition including a) about 1 to about 50 wt % of a mixture of barrier lipids consisting of cholesterol, one or more ceramides, and one or more free fatty acids, preferably about 1 to about 20 wt %, more preferably about 2 to about 10 wt %; b) about 0.1 to about 10 wt % of a skin conditioner, preferably about 1 to about 5 wt %, more preferably about 2 to about 4 wt %; c) about 0.001 to about 5 wt % of a chelating agent, preferably about 0.01 to about 2 wt %, more preferably about 0.1 to about 1 wt %; d) about 1 to about 30 wt % of a humectant, preferably about 3 to about 20 wt %, more preferably about 5 to about 10 wt %; e) about 0.5 to about 20 wt % of glyceryl stearate PEG-100 emulsifier, preferably about 0.5 to about 10 wt %, more preferably about 1 to about 5 wt %; f) about 1 to about 30 wt % of a mixture of emollients consisting of petrolatum and squalane, preferably about 2 to about 20 wt %, more preferably about 4 to about 10 wt %; g) about 0.1 to about 10 wt % of a mixture of preservatives consisting of phenoxyethanol and sorbic acid, preferably about 0.5 to about 5 wt %, more preferably about 0.8 to about 1.5 wt %; h) about 0.01 to about 5 wt % of citric acid, preferably about 0.25 to about 3 wt %, more preferably about 0.5 to about 2 wt %; i) about 1 to about 25 wt % of a mixture of encapsulation aids consisting of corn syrup solids and euphorbia cerifera (candelilla) wax, preferably about 5 to about 15 wt %, more preferably about 8 to about 10 wt %; j) about 0.01 to about 3 wt % of an aqueous phase thickener, preferably about 0.1 to about 2 wt %, more preferably about 0.2 to about 1 wt %; k) about 0.001 to about 50 wt % of hesperidin or an analog, pharmaceutically acceptable salt, or prodrug thereof, preferably about 1 to about 10 wt %, more preferably about 2 to about 5 wt %; and l) water. In embodiments, the topical composition is capable of treating dry skin.

Another aspect of the invention is directed to a composition including a) a mixture of optimized molar ratio of barrier lipids selected from the group consisting of cholesterol, ceramides, free fatty acids and mixtures of two or more thereof, in a total of about 1 to about 50 wt %, preferably about 1 to about 20 wt %, more preferably about 2 to about 10 wt %; b) about 0.001 to about 50 wt % of citrus bioflavonoid (e.g., hesperidin, or an analog, pharmaceutically acceptable salt, or prodrug thereof), preferably about 1 to about 10 wt %, more preferably about 2 to about 5 wt %; c) about 0.01 to about 5 wt % of an acid buffer, preferably about 0.25 to about 3 wt %, more preferably about 0.5 to about 2 wt %; and d) about 0.001% to about 5% of a topical glucocorticoid. In some embodiments, the glucocorticoid is selected from the group consisting of potent fluorinated agents that are most likely to provoke changes that mimic aged skin. In some embodiments, the citrus bioflavonoid (e.g., hesperidin, or an analog, pharmaceutically acceptable salt, or prodrug thereof) can be contained within its natural source, such as orange rind or peppermint oil, in an amount sufficient to provide the required wt % of the active citrus bioflavonoid (e.g., hesperidin, or an analog, pharmaceutically acceptable salt, or prodrug thereof).

Another aspect of the invention is directed to a composition including a) a mixture of optimized molar ratio of barrier lipids selected from the group consisting of cholesterol, ceramides, free fatty acids and mixtures of two or more thereof, in a total of about 1 to about 50 wt %, preferably about 1 to about 20 wt %, more preferably about 2 to about 10 wt %; b) about 0.001 to about 50 wt % of hesperidin, preferably about 1 to about 10 wt %, more preferably about 2 to about 5 wt %; c) about 0.01 to about 5 wt % of an acid buffer, preferably about 0.25 to about 3 wt %, more preferably about 0.5 to about 2 wt %; and d) about 0.001% to about 5% of a topical glucocorticoid. In some embodiments, the glucocorticoid is selected from the group consisting of potent fluorinated agents that are most likely to provoke changes that mimic aged skin. In some embodiments, the hesperidin can be contained within its natural source, such as orange rind or peppermint oil, in an amount sufficient to provide the required wt % of the active hesperidin.

Another aspect of the invention is directed to an anti-inflammatory composition including a) a mixture of optimized molar ratio of barrier lipids selected from the group consisting of cholesterol, ceramides, free fatty acids and mixtures of two or more thereof, in a total of about 1 to about 50 wt %, preferably about 1 to about 20 wt %, more preferably about 2 to about 10 wt %; b) about 0.001 to about 50 wt % of citrus bioflavonoid (e.g., hesperidin, or an analog, pharmaceutically acceptable salt, or prodrug thereof) or an analog, pharmaceutically acceptable salt, or prodrug thereof, preferably about 1 to about 10 wt %, more preferably about 2 to about 5 wt %; c) about 0.01 to about 5 wt % of an acid buffer, preferably about 0.25 to about 3 wt %, more preferably about 0.5 to about 2 wt %; and d) about 0.001% to about 5% of a topical glucocorticoid. In some embodiments, the glucocorticoid is selected from the group consisting of potent fluorinated agents that are most likely to provoke changes that mimic aged skin. In some embodiments, the citrus bioflavonoid (e.g., hesperidin, or an analog, pharmaceutically acceptable salt, or prodrug thereof) can be contained within its natural source, such as orange rind or peppermint oil, in an amount sufficient to provide the required wt % of the active citrus bioflavonoid (e.g., hesperidin, or an analog, pharmaceutically acceptable salt, or prodrug thereof). In embodiments, the citrus bioflavonoid is hesperidin.

Another aspect of the invention is directed to an anti-inflammatory composition including a) a mixture of optimized molar ratio of barrier lipids selected from the group consisting of cholesterol, ceramides, free fatty acids and mixtures of two or more thereof, in a total of about 1 to about 50 wt %, preferably about 1 to about 20 wt %, more preferably about 2 to about 10 wt %; b) about 0.001 to about 50 wt % of hesperidin or an analog, pharmaceutically acceptable salt, or prodrug thereof, preferably about 1 to about 10 wt %, more preferably about 2 to about 5 wt %; c) about 0.01 to about 5 wt % of an acid buffer, preferably about 0.25 to about 3 wt %, more preferably about 0.5 to about 2 wt %; and d) about 0.001% to about 5% of a topical glucocorticoid. In some embodiments, the glucocorticoid is selected from the group consisting of potent fluorinated agents that are most likely to provoke changes that mimic aged skin. In some embodiments, the hesperidin can be contained within its natural source, such as orange rind or peppermint oil, in an amount sufficient to provide the required wt % of the active hesperidin.

In embodiments, the glucocorticoid is present in about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, or about 5 wt %. In embodiments, the glucocorticoid is present in about 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2.0, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3, 2.31, 2.32, 2.33, 2.34, 2.35, 2.36, 2.37, 2.38, 2.39, 2.4, 2.41, 2.42, 2.43, 2.44, 2.45, 2.46, 2.47, 2.48, 2.49, 2.5, 2.51, 2.52, 2.53, 2.54, 2.55, 2.56, 2.57, 2.58, 2.59, 2.6, 2.61, 2.62, 2.63, 2.64, 2.65, 2.66, 2.67, 2.68, 2.69, 2.7, 2.71, 2.72, 2.73, 2.74, 2.75, 2.76, 2.77, 2.78, 2.79, 2.8, 2.81, 2.82, 2.83, 2.84, 2.85, 2.86, 2.87, 2.88, 2.89, 2.9, 2.91, 2.92, 2.93, 2.94, 2.95, 2.96, 2.97, 2.98, 2.99, or about 3.0 wt %.

One aspect of the invention is directed to a topical composition, the composition consisting of a) 1 to 50 wt % of a mixture of barrier lipids consisting of cholesterol, one or more ceramides, and one or more free fatty acids, preferably 1 to 20 wt %, more preferably 2 to 10 wt %; b) 0.1 to 10 wt % of a skin conditioner, preferably 1 to 5 wt %, more preferably 2 to 4 wt %; c) 0.001 to 5 wt % of a chelating agent, preferably 0.01 to 2 wt %, more preferably 0.1 to 1 wt %;

d) 1 to 30 wt % of a humectant, preferably 3 to 20 wt %, more preferably 5 to 10 wt %; e) 0.5 to 20 wt % of glyceryl stearate PEG-100 emulsifier, preferably 0.5 to 10 wt %, more preferably 1 to 5 wt %; f) 1 to 30 wt % of a mixture of emollients consisting of petrolatum and squalane, preferably 2 to 20 wt %, more preferably 4 to 10 wt %; g) 0.1 to 10 wt % of a mixture of preservatives consisting of phenoxyethanol and sorbic acid, preferably 0.5 to 5 wt %, more preferably 0.8 to 1.5 wt %; h) 0.01 to 5 wt % of citric acid, preferably 0.25 to 3 wt %, more preferably 0.5 to 2 wt %; i) 1 to 25 wt % of a mixture of encapsulation aids consisting of corn syrup solids and euphorbia cerifera (candelilla) wax, preferably 5 to 15 wt %, more preferably 8 to 10 wt %; j) 0.01 to 3 wt % of an aqueous phase thickener, preferably 0.1 to 2 wt %, more preferably 0.2 to 1 wt %; k) 0.001 to 50 wt % of citrus bioflavonoid (e.g., hesperidin, or an analog, pharmaceutically acceptable salt, or prodrug thereof), preferably 1 to 10 wt %, more preferably 2 to 5 wt %; and l) water. In embodiments, the citrus bioflavonoid is hesperidin.

In embodiments, citrus bioflavonoid (e.g., hesperidin, or an analog, pharmaceutically acceptable salt, or prodrug thereof) is present in 2 wt %. In embodiments, citrus bioflavonoid (e.g., hesperidin, or an analog, pharmaceutically acceptable salt, or prodrug thereof) is present in 5 wt %. In embodiments, citrus bioflavonoid (e.g., hesperidin, or an analog, pharmaceutically acceptable salt, or prodrug thereof) is present in 10 wt %. In embodiments, citrus bioflavonoid (e.g., hesperidin, or an analog, pharmaceutically acceptable salt, or prodrug thereof) is present in 15 wt %. In embodiments, citrus bioflavonoid (e.g., hesperidin, or an analog, pharmaceutically acceptable salt, or prodrug thereof) is present in 20 wt %. In embodiments, citrus bioflavonoid (e.g., hesperidin, or an analog, pharmaceutically acceptable salt, or prodrug thereof) is present in 25 wt %. In embodiments, citrus bioflavonoid (e.g., hesperidin, or an analog, pharmaceutically acceptable salt, or prodrug thereof) is present in 30 wt %. In embodiments, citrus bioflavonoid (e.g., hesperidin, or an analog, pharmaceutically acceptable salt, or prodrug thereof) is present in 35 wt %. In embodiments, the citrus bioflavonoid (e.g., hesperidin, or an analog, pharmaceutically acceptable salt, or prodrug thereof) can be contained within its natural source, such as orange rind or peppermint oil, in an amount sufficient to provide the required wt % of the active citrus bioflavonoid (e.g., hesperidin, or an analog, pharmaceutically acceptable salt, or prodrug thereof). The aqueous phase thickener can be, for example, xanthan gum.

One aspect of the invention is directed to a topical composition, the composition including a) 1 to 50 wt % of a mixture of barrier lipids consisting of cholesterol, one or more ceramides, and one or more free fatty acids, preferably 1 to 20 wt %, more preferably 2 to 10 wt %; b) 0.1 to 10 wt % of a skin conditioner, preferably 1 to 5 wt %, more preferably 2 to 4 wt %; c) 0.001 to 5 wt % of a chelating agent, preferably 0.01 to 2 wt %, more preferably 0.1 to 1 wt %; d) 1 to 30 wt % of a humectant, preferably 3 to 20 wt %, more preferably 5 to 10 wt %; e) 0.5 to 20 wt % of glyceryl stearate PEG-100 emulsifier, preferably 0.5 to 10 wt %, more preferably 1 to 5 wt %; f) 1 to 30 wt % of a mixture of emollients consisting of petrolatum and squalane, preferably 2 to 20 wt %, more preferably 4 to 10 wt %; g) 0.1 to 10 wt % of a mixture of preservatives consisting of phenoxyethanol and sorbic acid, preferably 0.5 to 5 wt %, more preferably 0.8 to 1.5 wt %; h) 0.01 to 5 wt % of citric acid, preferably 0.25 to 3 wt %, more preferably 0.5 to 2 wt %; i) 1 to 25 wt % of a mixture of encapsulation aids consisting of corn syrup solids and euphorbia cerifera (candelilla) wax, preferably 5 to 15 wt %, more preferably 8 to 10 wt %; j) 0.01 to 3 wt % of an aqueous phase thickener, preferably 0.1 to 2 wt %, more preferably 0.2 to 1 wt %; k) 0.001 to 50 wt % of citrus bioflavonoid (e.g., hesperidin, or an analog, pharmaceutically acceptable salt, or prodrug thereof), preferably 1 to 10 wt %, more preferably 2 to 5 wt %; and l) water. In embodiments, the topical composition is capable of treating dry skin. In embodiments, the citrus bioflavonoid 15 hesperidin.

Another aspect of the invention is directed to a composition including a) a mixture of optimized molar ratio of barrier lipids selected from the group consisting of cholesterol, ceramides, free fatty acids and mixtures of two or more thereof, in a total of 1 to 50 wt %, preferably 1 to 20 wt %, more preferably 2 to 10 wt %; b) 0.001 to 50 wt % of citrus bioflavonoid (e.g., hesperidin, or an analog, pharmaceutically acceptable salt, or prodrug thereof), preferably 1 to 10 wt %, more preferably 2 to 5 wt %; c) 0.01 to 5 wt % of an acid buffer, preferably 0.25 to 3 wt %, more preferably 0.5 to 2 wt %; and d) 0.001% to 5% of a topical glucocorticoid. In some embodiments, the glucocorticoid is selected from the group consisting of potent fluorinated agents that are most likely to provoke changes that mimic aged skin. In some embodiments, the citrus bioflavonoid (e.g., hesperidin, or an analog, pharmaceutically acceptable salt, or prodrug thereof) can be contained within its natural source, such as orange rind or peppermint oil, in an amount sufficient to provide the required wt % of the active citrus bioflavonoid (e.g., hesperidin, or an analog, pharmaceutically acceptable salt, or prodrug thereof). In embodiments, the citrus bioflavonoid is hesperidin.

Another aspect of the invention is directed to an anti-inflammatory composition including a) a mixture of optimized molar ratio of barrier lipids selected from the group consisting of cholesterol, ceramides, free fatty acids and mixtures of two or more thereof, in a total of 1 to 50 wt %, preferably 1 to 20 wt %, more preferably 2 to 10 wt %; b) 0.001 to 50 wt % of citrus bioflavonoid (e.g., hesperidin, or an analog, pharmaceutically acceptable salt, or prodrug thereof) or an analog, pharmaceutically acceptable salt, or prodrug thereof, preferably 1 to 10 wt %, more preferably 2 to 5 wt %; c) 0.01 to 5 wt % of an acid buffer, preferably 0.25 to 3 wt %, more preferably 0.5 to 2 wt %; and d) 0.001% to 5% of a topical glucocorticoid. In some embodiments, the glucocorticoid is selected from the group consisting of potent fluorinated agents that are most likely to provoke changes that mimic aged skin. In some embodiments, the citrus bioflavonoid (e.g., hesperidin, or an analog, pharmaceutically acceptable salt, or prodrug thereof) can be contained within its natural source, such as orange rind or peppermint oil, in an amount sufficient to provide the required wt % of the active citrus bioflavonoid (e.g., hesperidin, or an analog, pharmaceutically acceptable salt, or prodrug thereof). In embodiments, the citrus bioflavonoid is hesperidin.

One aspect of the invention is directed to a topical composition, the composition consisting of a) 1 to 50 wt % of a mixture of barrier lipids consisting of cholesterol, one or more ceramides, and one or more free fatty acids, preferably 1 to 20 wt %, more preferably 2 to 10 wt %; b) 0.1 to 10 wt % of a skin conditioner, preferably 1 to 5 wt %, more preferably 2 to 4 wt %; c) 0.001 to 5 wt % of a chelating agent, preferably 0.01 to 2 wt %, more preferably 0.1 to 1 wt %; d) 1 to 30 wt % of a humectant, preferably 3 to 20 wt %, more preferably 5 to 10 wt %; e) 0.5 to 20 wt % of glyceryl stearate PEG-100 emulsifier, preferably 0.5 to 10 wt %, more preferably 1 to 5 wt %; f) 1 to 30 wt % of a mixture of emollients consisting of petrolatum and squalane, preferably 2 to 20 wt %, more preferably 4 to 10 wt %; g) 0.1 to 10 wt % of a mixture of preservatives consisting of phenoxyethanol and sorbic acid, preferably 0.5 to 5 wt %, more preferably 0.8 to 1.5 wt %; h) 0.01 to 5 wt % of citric acid, preferably 0.25 to 3 wt %, more preferably 0.5 to 2 wt %; i) 1 to 25 wt % of a mixture of encapsulation aids consisting of corn syrup solids and euphorbia cerifera (candelilla) wax, preferably 5 to 15 wt %, more preferably 8 to 10 wt %; j) 0.01 to 3 wt % of an aqueous phase thickener, preferably 0.1 to 2 wt %, more preferably 0.2 to 1 wt %; k) 0.001 to 50 wt % of hesperidin or an analog, pharmaceutically acceptable salt, or prodrug thereof, preferably 1 to 10 wt %, more preferably 2 to 5 wt %; and l) water.

In embodiments, hesperidin or an analog, pharmaceutically acceptable salt, or prodrug thereof is present in 2 wt %. In embodiments, hesperidin or an analog, pharmaceutically acceptable salt, or prodrug thereof is present in 5 wt %. In embodiments, hesperidin or an analog, pharmaceutically acceptable salt, or prodrug thereof is present in 10 wt %. In embodiments, hesperidin or an analog, pharmaceutically acceptable salt, or prodrug thereof is present in 15 wt %. In embodiments, hesperidin or an analog, pharmaceutically acceptable salt, or prodrug thereof is present in 20 wt %. In embodiments, hesperidin or an analog, pharmaceutically acceptable salt, or prodrug thereof is present in 25 wt %. In embodiments, hesperidin or an analog, pharmaceutically acceptable salt, or prodrug thereof is present in 30 wt %. In embodiments, hesperidin or an analog, pharmaceutically acceptable salt, or prodrug thereof is present in 35 wt %. In embodiments, the hesperidin or an analog, pharmaceutically acceptable salt, or prodrug thereof can be contained within its natural source, such as orange rind or peppermint oil, in an amount sufficient to provide the required wt % of the active hesperidin or an analog, pharmaceutically acceptable salt, or prodrug thereof.

One aspect of the invention is directed to a topical composition, the composition consisting of a) 1 to 50 wt % of a mixture of barrier lipids consisting of cholesterol, one or more ceramides, and one or more free fatty acids, preferably 1 to 20 wt %, more preferably 2 to 10 wt %; b) 0.1 to 10 wt % of a skin conditioner, preferably 1 to 5 wt %, more preferably 2 to 4 wt %; c) 0.001 to 5 wt % of a chelating agent, preferably 0.01 to 2 wt %, more preferably 0.1 to 1 wt %; d) 1 to 30 wt % of a humectant, preferably 3 to 20 wt %, more preferably 5 to 10 wt %; e) 0.5 to 20 wt % of glyceryl stearate PEG-100 emulsifier, preferably 0.5 to 10 wt %, more preferably 1 to 5 wt %; f) 1 to 30 wt % of a mixture of emollients consisting of petrolatum and squalane, preferably 2 to 20 wt %, more preferably 4 to 10 wt %; g) 0.1 to 10 wt % of a mixture of preservatives consisting of phenoxyethanol and sorbic acid, preferably 0.5 to 5 wt %, more preferably 0.8 to 1.5 wt %; h) 0.01 to 5 wt % of citric acid, preferably 0.25 to 3 wt %, more preferably 0.5 to 2 wt %; i) 1 to 25 wt % of a mixture of encapsulation aids consisting of corn syrup solids and euphorbia cerifera (candelilla) wax, preferably 5 to 15 wt %, more preferably 8 to 10 wt %; j) 0.01 to 3 wt % of an aqueous phase thickener, preferably 0.1 to 2 wt %, more preferably 0.2 to 1 wt %; k) 0.001 to 50 wt % of hesperidin, preferably 1 to 10 wt %, more preferably 2 to 5 wt %; and l) water.

In embodiments, hesperidin is present in 2 wt %. In embodiments, hesperidin is present in 5 wt %. In embodiments, hesperidin is present in 10 wt %. In embodiments, hesperidin is present in 15 wt %. In embodiments, hesperidin is present in 20 wt %. In embodiments, hesperidin is present in 25 wt %. In embodiments, hesperidin is present in 30 wt %. In embodiments, hesperidin is present in 35 wt %. In embodiments, the hesperidin can be contained within its natural source, such as orange rind or peppermint oil, in an amount sufficient to provide the required wt % of the active hesperidin.

One aspect of the invention is directed to a topical composition, the composition including a) 1 to 50 wt % of a mixture of barrier lipids consisting of cholesterol, one or more ceramides, and one or more free fatty acids, preferably 1 to 20 wt %, more preferably 2 to 10 wt %; b) 0.1 to 10 wt % of a skin conditioner, preferably 1 to 5 wt %, more preferably 2 to 4 wt %; c) 0.001 to 5 wt % of a chelating agent, preferably 0.01 to 2 wt %, more preferably 0.1 to 1 wt %; d) 1 to 30 wt % of a humectant, preferably 3 to 20 wt %, more preferably 5 to 10 wt %; e) 0.5 to 20 wt % of glyceryl stearate PEG-100 emulsifier, preferably 0.5 to 10 wt %, more preferably 1 to 5 wt %; f) 1 to 30 wt % of a mixture of emollients consisting of petrolatum and squalane, preferably 2 to 20 wt %, more preferably 4 to 10 wt %; g) 0.1 to 10 wt % of a mixture of preservatives consisting of phenoxyethanol and sorbic acid, preferably 0.5 to 5 wt %, more preferably 0.8 to 1.5 wt %; h) 0.01 to 5 wt % of citric acid, preferably 0.25 to 3 wt %, more preferably 0.5 to 2 wt %; i) 1 to 25 wt % of a mixture of encapsulation aids consisting of corn syrup solids and euphorbia cerifera (candelilla) wax, preferably 5 to 15 wt %, more preferably 8 to 10 wt %; j) 0.01 to 3 wt % of an aqueous phase thickener, preferably 0.1 to 2 wt %, more preferably 0.2 to 1 wt %; k) 0.001 to 50 wt % of hesperidin or an analog, pharmaceutically acceptable salt, or prodrug thereof, preferably 1 to 10 wt %, more preferably 2 to 5 wt %; and l) water. In embodiments, the topical composition is capable of treating dry skin.

Another aspect of the invention is directed to a composition including a) a mixture of optimized molar ratio of barrier lipids selected from the group consisting of cholesterol, ceramides, free fatty acids and mixtures of two or more thereof, in a total of 1 to 50 wt %, preferably 1 to 20 wt %, more preferably 2 to 10 wt %; b) 0.001 to 50 wt % of hesperidin, preferably 1 to 10 wt %, more preferably 2 to 5 wt %; c) 0.01 to 5 wt % of an acid buffer, preferably 0.25 to 3 wt %, more preferably 0.5 to 2 wt %; and d) 0.001% to 5% of a topical glucocorticoid. In some embodiments, the glucocorticoid is selected from the group consisting of potent fluorinated agents that are most likely to provoke changes that mimic aged skin. In some embodiments, the hesperidin can be contained within its natural source, such as orange rind or peppermint oil, in an amount sufficient to provide the required wt % of the active hesperidin.

Another aspect of the invention is directed to an anti-inflammatory composition including a) a mixture of optimized molar ratio of barrier lipids selected from the group consisting of cholesterol, ceramides, free fatty acids and mixtures of two or more thereof, in a total of 1 to 50 wt %, preferably 1 to 20 wt %, more preferably 2 to 10 wt %; b) 0.001 to 50 wt % of hesperidin or an analog, pharmaceutically acceptable salt, or prodrug thereof, preferably 1 to 10 wt %, more preferably 2 to 5 wt %; c) 0.01 to 5 wt % of an acid buffer, preferably 0.25 to 3 wt %, more preferably 0.5 to 2 wt %; and d) 0.001% to 5% of a topical glucocorticoid. In some embodiments, the glucocorticoid is selected from the group consisting of potent fluorinated agents that are most likely to provoke changes that mimic aged skin. In some embodiments, the hesperidin or an analog, pharmaceutically acceptable salt, or prodrug thereof can be contained within its natural source, such as orange rind or peppermint oil, in an amount sufficient to provide the required wt % of the active hesperidin or an analog, pharmaceutically acceptable salt, or prodrug thereof.

Another aspect of the invention is directed to an anti-inflammatory composition including a) a mixture of optimized molar ratio of barrier lipids selected from the group consisting of cholesterol, ceramides, free fatty acids and mixtures of two or more thereof, in a total of 1 to 50 wt %, preferably 1 to 20 wt %, more preferably 2 to 10 wt %; b) 0.001 to 50 wt % of hesperidin, preferably 1 to 10 wt %, more preferably 2 to 5 wt %; c) 0.01 to 5 wt % of an acid buffer, preferably 0.25 to 3 wt %, more preferably 0.5 to 2 wt %; and d) 0.001% to 5% of a topical glucocorticoid. In some embodiments, the glucocorticoid is selected from the group consisting of potent fluorinated agents that are most likely to provoke changes that mimic aged skin. In some embodiments, the hesperidin can be contained within its natural source, such as orange rind or peppermint oil, in an amount sufficient to provide the required wt % of the active hesperidin.

In embodiments, the glucocorticoid is present in 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, or 5 wt %. In embodiments, the glucocorticoid is present in 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2.0, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3, 2.31, 2.32, 2.33, 2.34, 2.35, 2.36, 2.37, 2.38, 2.39, 2.4, 2.41, 2.42, 2.43, 2.44, 2.45, 2.46, 2.47, 2.48, 2.49, 2.5, 2.51, 2.52, 2.53, 2.54, 2.55, 2.56, 2.57, 2.58, 2.59, 2.6, 2.61, 2.62, 2.63, 2.64, 2.65, 2.66, 2.67, 2.68, 2.69, 2.7, 2.71, 2.72, 2.73, 2.74, 2.75, 2.76, 2.77, 2.78, 2.79, 2.8, 2.81, 2.82, 2.83, 2.84, 2.85, 2.86, 2.87, 2.88, 2.89, 2.9, 2.91, 2.92, 2.93, 2.94, 2.95, 2.96, 2.97, 2.98, 2.99, or 3.0 wt %.

The free fatty acid and/or cholesterol components of the above compositions can also include lipid metabolites or naturally-occurring oils enriched in species that are known activators or ligands for the nuclear hormone receptors PPARα, PPARβ/δ and/or LXR. Such lipid metabolites include, for example, the PPARα activator, gamma-linoleic acid. The free fatty acid can be selected from either essential or non-essential free fatty acids, or can be a combination thereof.

The topical compositions of the invention can be adapted for therapeutic applications to treat various skin diseases and disorders, and also can be adapted to cosmetic uses. The topical compositions can contain a wide variety of optional components (e.g., scents); provided that such optional components are physically and chemically compatible with the essential components described herein, and do not unduly impair stability, efficacy, or other use benefits associated with the compositions. Optional components can be dispersed, dissolved, or the like in the carrier of the present compositions.

Exemplary optional components include emollients, oil absorbents, antimicrobial agents, binders, additional buffering agents, denaturants, cosmetic astringents, external analgesics, film formers, humectants, opacifying agents, perfumes, pigments, skin soothing and healing agents, preservatives, propellants, skin penetration enhancers, solvents, suspending agents, emulsifiers, cleansing agents, thickening agents, solubilizing agents, waxes, sunscreens, sunless tanning agents, antioxidants and/or radical scavengers, chelating agents, anti-acne agents, anti-inflammatory agents, desquamation agents/exfoliants, organic hydroxy acids, vitamins, and natural extracts. Examples of such materials are described in Harry's Cosmeticology, 7th Ed., Harry & Wilkinson (Hill Publishers, London 1982); in Pharmaceutical Dosage Forms—Disperse Systems; Lieberman, Rieger & Banker, Vols. 1 (1988) & 2 (1989); Marcel Decker, Inc.; in The Chemistry and Manufacture of Cosmetics, 2nd. Ed., deNavarre (Van Nostrand 1962-1965); and in The Handbook of Cosmetic Science and Technology, 1st Ed. Knowlton & Pearce (Elsevier 1993) can also be used in the present invention.

In an aspect is provided a composition including citrus bioflavonoid (e.g., hesperidin, or an analog, pharmaceutically acceptable salt, or prodrug thereof). In an aspect is provided a composition including cholesterol and citrus bioflavonoid (e.g., hesperidin, or an analog, pharmaceutically acceptable salt, or prodrug thereof). In embodiments, the citrus bioflavonoid is hesperidin. In embodiments, the composition further includes a ceramide and a free fatty acid. In embodiments, the molar ratio of total cholesterol:total ceramide:total free fatty acid is about 3:1:1 molar. In embodiments, the molar ratio of total cholesterol:total ceramide:total free fatty acid is 3:1:1 molar.

In an aspect is provided a composition including hesperidin. In an aspect is provided a composition including cholesterol and hesperidin. In embodiments, the composition further includes a ceramide and a free fatty acid. In embodiments, the molar ratio of total cholesterol:total ceramide:total free fatty acid is about 3:1:1 molar. In embodiments, the molar ratio of total cholesterol:total ceramide:total free fatty acid is 3:1:1 molar.

In embodiments, the composition further includes an acid buffer. In embodiments, the pH of the composition is between about 3 to about 5.5. In embodiments, the composition includes about 1 to about 50 wt % of total cholesterol. In embodiments, the composition includes about 0.1 to about 10 wt % of total cholesterol. In embodiments, the composition includes about 0.1 to about 5 wt % of total cholesterol. In embodiments, the composition includes about 0.001 to about 50 wt % of hesperidin. In embodiments, the composition includes about 0.1 to about 10 wt % of hesperidin. In embodiments, the composition includes about 0.1 to about 5 wt % of hesperidin. In embodiments, the composition includes about 0.1 to about 10 wt % total ceramide. In embodiments, the composition includes about 0.1 to about 5 wt % total ceramide. In embodiments, the composition includes about 0.01 to about 6 wt % total free fatty acid. In embodiments, the composition includes about 0.1 to about 2 wt % total free fatty acid. In embodiments, the composition includes about 0.05 to about 5 wt % of the acid buffer. In embodiments, the composition includes about 0.1 to about 5 wt % of the acid buffer.

In embodiments, the composition further includes a skin conditioner. In embodiments, the composition includes about 0.1 to about 10 wt % of the skin conditioner. In embodiments, the composition includes about 0.1 to about 5 wt % of the skin conditioner. In embodiments, the skin conditioner is dimethicone. In embodiments, the composition further includes a chelating agent. In embodiments, the composition includes about 0.001 to about 5 wt % of the chelating agent. In embodiments, the composition includes about 0.01 to about 1 wt % of the chelating agent. In embodiments, the chelating agent is disodium EDTA. In embodiments, the composition further includes a humectant. In embodiments, the composition includes about 1 to about 30 wt % of the humectant. In embodiments, the composition includes about 5 to about 30 wt % of the humectant. In embodiments, the humectant is glycerin. In embodiments, the composition further includes an emulsifier. In embodiments, the composition includes about 0.5 to about 20 wt % of the emulsifier. In embodiments, the composition includes about 0.2 to about 10 wt % of the emulsifier. In embodiments, the emulsifier is glyceryl stearate PEG-100. In embodiments, the composition further includes an emollient. In embodiments, the composition includes about 1 to about 30 wt % of the emollient. In embodiments, the composition includes about 0.2 to about 10 wt % of the emollient. In embodiments, the emollient is petrolatum. In embodiments, the emollient is squalane. In embodiments, the composition further includes a preservative. In embodiments, the composition includes about 0.1 to about 10 wt % of the preservative. In embodiments, the composition includes about 0.01 to about 5 wt % of the preservative. In embodiments, the preservative is phenoxyethanol. In embodiments, the preservative is sorbic acid. In embodiments, the composition further includes an encapsulation aid. In embodiments, the composition includes about 1 to about 25 wt % of the encapsulation aid. In embodiments, the composition includes about 0.01 to about 30 wt % of the encapsulation aid. In embodiments, the encapsulation aid is euphorbia cerifera (candelilla) wax. In embodiments, the encapsulation aid is corn syrup solids. In embodiments, the composition further includes an aqueous phase thickener. In embodiments, the composition includes about 0.01 to about 3 wt % of the aqueous phase thickener. In embodiments, the aqueous phase thickener is xanthan gum. In embodiments, the ceramide is hydroxypropyl bispalmitamide MEA (Ceramide PC-104). In embodiments, the free fatty acid is conjugated linoleic acid (CLA). In embodiments, the composition further includes a glucocorticoid. In embodiments, the composition includes about 0.001 to about 5 wt % of the glucocorticoid. In embodiments, the composition is a topical composition.

In embodiments is provided a topical composition, the composition including: a) cholesterol (e.g., about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or about 3.0 wt %); b) an acid buffer (e.g., 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.55, 0.6, 0.65, 0.7, 0.75, 0.8, 0.9, 1, 2, 3, 4, 5, 6, 7, 8, 9, 10, 11, 12, 13, 14, 15, 16, 17, 18, 19, 20, 21, 2, 23, 24, 25, 26, 27, 28, 29, 30, 31, 32, 33, 34, 35, 36, 37, 38, 39, 40, 41, 42, 43, 44, 45, 46, 47, 48, 49 or about 50 wt %); and c) hesperidin (e.g., about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or about 3.0 wt %); wherein the pH of the topical composition is about 3 to about 5.5.

In embodiments is provided a topical composition, the composition including: a) a mixture of barrier lipids selected from the group consisting of cholesterol, at least one ceramide, at least one free fatty acid, and mixtures of two or more thereof (e.g., about 1.0, 1.1, 1.2, 1.3, 1.4, 1.5, 1.6, 1.7, 1.8, 1.9, 2.0, 2.02, 2.04, 2.06, 2.08, 2.1, 2.12, 2.14, 2.16, 2.18, 2.2, 2.22, 2.24, 2.26, 2.28, 2.3, 2.32, 2.34, 2.36, 2.38, 2.4, 2.42, 2.44, 2.46, 2.48, 2.5, 2.52, 2.54, 2.56, 2.58, 2.6, 2.62, 2.64, 2.66, 2.68, 2.7, 2.72, 2.74, 2.76, 2.78, 2.8, 2.82, 2.84, 2.86, 2.88, 2.9, 2.92, 2.94, 2.96, 2.98, 3.0, 3.02, 3.04, 3.06, 3.08, 3.1, 3.12, 3.14, 3.16, 3.18, 3.2, 3.22, 3.24, 3.26, 3.28, 3.3, 3.32, 3.34, 3.36, 3.38, 3.4, 3.42, 3.44, 3.46, 3.48, 3.5, 3.52, 3.54, 3.56, 3.58, 3.6, 3.62, 3.64, 3.66, 3.68, 3.7, 3.72, 3.74, 3.76, 3.78, 3.8, 3.82, 3.84, 3.86, 3.88, 3.9, 3.92, 3.94, 3.96, 3.98, 4.0, 4.02, 4.04, 4.06, 4.08, 4.1, 4.12, 4.14, 4.16, 4.18, 4.2, 4.22, 4.24, 4.26, 4.28, 4.3, 4.32, 4.34, 4.36, 4.38, 4.4, 4.42, 4.44, 4.46, 4.48, 4.5, 4.52, 4.54, 4.56, 4.58, 4.6, 4.62, 4.64, 4.66, 4.68, 4.7, 4.72, 4.74, 4.76, 4.78, 4.8, 4.82, 4.84, 4.86, 4.88, 4.9, 4.92, 4.94, 4.96, 4.98, 5.0, 5.02, 5.04, 5.06, 5.08, 5.1, 5.12, 5.14, 5.16, 5.18, 5.2, 5.22, 5.24, 5.26, 5.28, 5.3, 5.32, 5.34, 5.36, 5.38, 5.4, 5.42, 5.44, 5.46, 5.48, 5.5, 5.52, 5.54, 5.56, 5.58, 5.6, 5.62, 5.64, 5.66, 5.68, 5.7, 5.72, 5.74, 5.76, 5.78, 5.8, 5.82, 5.84, 5.86, 5.88, 5.9, 5.92, 5.94, 5.96, 5.98, 6.0, 6.02, 6.04, 6.06, 6.08, 6.1, 6.12, 6.14, 6.16, 6.18, 6.2, 6.22, 6.24, 6.26, 6.28, 6.3, 6.32, 6.34, 6.36, 6.38, 6.4, 6.42, 6.44, 6.46, 6.48, 6.5, 6.52, 6.54, 6.56, 6.58, 6.6, 6.62, 6.64, 6.66, 6.68, 6.7, 6.72, 6.74, 6.76, 6.78, 6.8, 6.82, 6.84, 6.86, 6.88, 6.9, 6.92, 6.94, 6.96, 6.98, 7.0, 7.02, 7.04, 7.06, 7.08, 7.1, 7.12, 7.14, 7.16, 7.18, 7.2, 7.22, 7.24, 7.26, 7.28, 7.3, 7.32, 7.34, 7.36, 7.38, 7.4, 7.42, 7.44, 7.46, 7.48, 7.5, 7.52, 7.54, 7.56, 7.58, 7.6, 7.62, 7.64, 7.66, 7.68, 7.7, 7.72, 7.74, 7.76, 7.78, 7.8, 7.82, 7.84, 7.86, 7.88, 7.9, 7.92, 7.94, 7.96, 7.98, 8.0, 8.02, 8.04, 8.06, 8.08, 8.1, 8.12, 8.14, 8.16, 8.18, 8.2, 8.22, 8.24, 8.26, 8.28, 8.3, 8.32, 8.34, 8.36, 8.38, 8.4, 8.42, 8.44, 8.46, 8.48, 8.5, 8.52, 8.54, 8.56, 8.58, 8.6, 8.62, 8.64, 8.66, 8.68, 8.7, 8.72, 8.74, 8.76, 8.78, 8.8, 8.82, 8.84, 8.86, 8.88, 8.9, 8.92, 8.94, 8.96, 8.98, 9.0, 9.02, 9.04, 9.06, 9.08, 9.1, 9.12, 9.14, 9.16, 9.18, 9.2, 9.22, 9.24, 9.26, 9.28, 9.3, 9.32, 9.34, 9.36, 9.38, 9.4, 9.42, 9.44, 9.46, 9.48, 9.5, 9.52, 9.54, 9.56, 9.58, 9.6, 9.62, 9.64, 9.66, 9.68, 9.7, 9.72, 9.74, 9.76, 9.78, 9.8, 9.82, 9.84, 9.86, 9.88, 9.9, 9.92, 9.94, 9.96, 9.98, or about 10 total wt %); b) a skin conditioner (e.g., about 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2.0, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3, 2.31, 2.32, 2.33, 2.34, 2.35, 2.36, 2.37, 2.38, 2.39, 2.4, 2.41, 2.42, 2.43, 2.44, 2.45, 2.46, 2.47, 2.48, 2.49, 2.5, 2.51, 2.52, 2.53, 2.54, 2.55, 2.56, 2.57, 2.58, 2.59, 2.6, 2.61, 2.62, 2.63, 2.64, 2.65, 2.66, 2.67, 2.68, 2.69, 2.7, 2.71, 2.72, 2.73, 2.74, 2.75, 2.76, 2.77, 2.78, 2.79, 2.8, 2.81, 2.82, 2.83, 2.84, 2.85, 2.86, 2.87, 2.88, 2.89, 2.9, 2.91, 2.92, 2.93, 2.94, 2.95, 2.96, 2.97, 2.98, 2.99, or about 3.0 wt %); c) a chelating agent (e.g., about 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or about 1.0 wt %); d) a humectant (e.g., about 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0, 25.0, 26.0, 27.0, 28.0, 29.0 or about 30.0 wt %); e) an emulsifier (about 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2.0, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3, 2.31, 2.32, 2.33, 2.34, 2.35, 2.36, 2.37, 2.38, 2.39, 2.4, 2.41, 2.42, 2.43, 2.44, 2.45, 2.46, 2.47, 2.48, 2.49, 2.5, 2.51, 2.52, 2.53, 2.54, 2.55, 2.56, 2.57, 2.58, 2.59, 2.6, 2.61, 2.62, 2.63, 2.64, 2.65, 2.66, 2.67, 2.68, 2.69, 2.7, 2.71, 2.72, 2.73, 2.74, 2.75, 2.76, 2.77, 2.78, 2.79, 2.8, 2.81, 2.82, 2.83, 2.84, 2.85, 2.86, 2.87, 2.88, 2.89, 2.9, 2.91, 2.92, 2.93, 2.94, 2.95, 2.96, 2.97, 2.98, 2.99, or about 3.0 wt %); f) one or more emollients (e.g., about 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24.0, 24.5, 25.0, 25.5, 26.0, 26.5, 27.0, 27.5, 28.0, 28.5, 29.0, 29.5, or about 30.0 wt %); g) one or more preservatives (e.g., about 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, or about total 1.5 wt %); h) an acid buffer (e.g., about 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.55, 0.6, 0.65, 0.7, 0.75, 0.8, 0.9, 1, 2, 3, 4, 5, 6, 7, 8, 9, 10, 11, 12, 13, 14, 15, 16, 17, 18, 19, 20, 21, 2, 23, 24, 25, 26, 27, 28, 29, 30, 31, 32, 33, 34, 35, 36, 37, 38, 39, 40, 41, 42, 43, 44, 45, 46, 47, 48, 49 or about 50 wt %); i) one or more encapsulation aids (e.g., about 7.0, 7.02, 7.04, 7.06, 7.08, 7.1, 7.12, 7.14, 7.16, 7.18, 7.2, 7.22, 7.24, 7.26, 7.28, 7.3, 7.32, 7.34, 7.36, 7.38, 7.4, 7.42, 7.44, 7.46, 7.48, 7.5, 7.52, 7.54, 7.56, 7.58, 7.6, 7.62, 7.64, 7.66, 7.68, 7.7, 7.72, 7.74, 7.76, 7.78, 7.8, 7.82, 7.84, 7.86, 7.88, 7.9, 7.92, 7.94, 7.96, 7.98, 8.0, 8.02, 8.04, 8.06, 8.08, 8.1, 8.12, 8.14, 8.16, 8.18, 8.2, 8.22, 8.24, 8.26, 8.28, 8.3, 8.32, 8.34, 8.36, 8.38, 8.4, 8.42, 8.44, 8.46, 8.48, 8.5, 8.52, 8.54, 8.56, 8.58, 8.6, 8.62, 8.64, 8.66, 8.68, 8.7, 8.72, 8.74, 8.76, 8.78, 8.8, 8.82, 8.84, 8.86, 8.88, 8.9, 8.92, 8.94, 8.96, 8.98, 9.0, 9.02, 9.04, 9.06, 9.08, 9.1, 9.12, 9.14, 9.16, 9.18, 9.2, 9.22, 9.24, 9.26, 9.28, 9.3, 9.32, 9.34, 9.36, 9.38, 9.4, 9.42, 9.44, 9.46, 9.48, 9.5, 9.52, 9.54, 9.56, 9.58, 9.6, 9.62, 9.64, 9.66, 9.68, 9.7, 9.72, 9.74, 9.76, 9.78, 9.8, 9.82, 9.84, 9.86, 9.88, 9.9, 9.92, 9.94, 9.96, 9.98, or about 10.0 total wt %; j) an aqueous phase thickener (e.g., about 0.1, 0.12, 0.14, 0.16, 0.18, 0.2, 0.22, 0.24, 0.26, 0.28, 0.3, 0.32, 0.34, 0.36, 0.38, 0.4, 0.42, 0.44, 0.46, 0.48, 0.5, 0.52, 0.54, 0.56, 0.58, 0.6, 0.62, 0.64, 0.66, 0.68, 0.7, 0.72, 0.74, 0.76, 0.78, 0.8, 0.82, 0.84, 0.86, 0.88, 0.9, 0.92, 0.94, 0.96, 0.98, 1.0, 1.02, 1.04, 1.06, 1.08, 1.1, 1.12, 1.14, 1.16, 1.18, 1.2, 1.22, 1.24, 1.26, 1.28, 1.3, 1.32, 1.34, 1.36, 1.38, 1.4, 1.42, 1.44, 1.46, 1.48, 1.5, 1.52, 1.54, 1.56, 1.58, 1.6, 1.62, 1.64, 1.66, 1.68, 1.7, 1.72, 1.74, 1.76, 1.78, 1.8, 1.82, 1.84, 1.86, 1.88, 1.9, 1.92, 1.94, 1.96, 1.98, or about 2.0 wt %); and k) hesperidin (e.g., about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or about 3.0 wt %).

In embodiments is provided a topical composition consisting of: a) a mixture of barrier lipids consisting of cholesterol, one or more ceramides, and one or more free fatty acids (e.g., about 1.0, 1.1, 1.2, 1.3, 1.4, 1.5, 1.6, 1.7, 1.8, 1.9, 2.0, 2.02, 2.04, 2.06, 2.08, 2.1, 2.12, 2.14, 2.16, 2.18, 2.2, 2.22, 2.24, 2.26, 2.28, 2.3, 2.32, 2.34, 2.36, 2.38, 2.4, 2.42, 2.44, 2.46, 2.48, 2.5, 2.52, 2.54, 2.56, 2.58, 2.6, 2.62, 2.64, 2.66, 2.68, 2.7, 2.72, 2.74, 2.76, 2.78, 2.8, 2.82, 2.84, 2.86, 2.88, 2.9, 2.92, 2.94, 2.96, 2.98, 3.0, 3.02, 3.04, 3.06, 3.08, 3.1, 3.12, 3.14, 3.16, 3.18, 3.2, 3.22, 3.24, 3.26, 3.28, 3.3, 3.32, 3.34, 3.36, 3.38, 3.4, 3.42, 3.44, 3.46, 3.48, 3.5, 3.52, 3.54, 3.56, 3.58, 3.6, 3.62, 3.64, 3.66, 3.68, 3.7, 3.72, 3.74, 3.76, 3.78, 3.8, 3.82, 3.84, 3.86, 3.88, 3.9, 3.92, 3.94, 3.96, 3.98, 4.0, 4.02, 4.04, 4.06, 4.08, 4.1, 4.12, 4.14, 4.16, 4.18, 4.2, 4.22, 4.24, 4.26, 4.28, 4.3, 4.32, 4.34, 4.36, 4.38, 4.4, 4.42, 4.44, 4.46, 4.48, 4.5, 4.52, 4.54, 4.56, 4.58, 4.6, 4.62, 4.64, 4.66, 4.68, 4.7, 4.72, 4.74, 4.76, 4.78, 4.8, 4.82, 4.84, 4.86, 4.88, 4.9, 4.92, 4.94, 4.96, 4.98, 5.0, 5.02, 5.04, 5.06, 5.08, 5.1, 5.12, 5.14, 5.16, 5.18, 5.2, 5.22, 5.24, 5.26, 5.28, 5.3, 5.32, 5.34, 5.36, 5.38, 5.4, 5.42, 5.44, 5.46, 5.48, 5.5, 5.52, 5.54, 5.56, 5.58, 5.6, 5.62, 5.64, 5.66, 5.68, 5.7, 5.72, 5.74, 5.76, 5.78, 5.8, 5.82, 5.84, 5.86, 5.88, 5.9, 5.92, 5.94, 5.96, 5.98, 6.0, 6.02, 6.04, 6.06, 6.08, 6.1, 6.12, 6.14, 6.16, 6.18, 6.2, 6.22, 6.24, 6.26, 6.28, 6.3, 6.32, 6.34, 6.36, 6.38, 6.4, 6.42, 6.44, 6.46, 6.48, 6.5, 6.52, 6.54, 6.56, 6.58, 6.6, 6.62, 6.64, 6.66, 6.68, 6.7, 6.72, 6.74, 6.76, 6.78, 6.8, 6.82, 6.84, 6.86, 6.88, 6.9, 6.92, 6.94, 6.96, 6.98, 7.0, 7.02, 7.04, 7.06, 7.08, 7.1, 7.12, 7.14, 7.16, 7.18, 7.2, 7.22, 7.24, 7.26, 7.28, 7.3, 7.32, 7.34, 7.36, 7.38, 7.4, 7.42, 7.44, 7.46, 7.48, 7.5, 7.52, 7.54, 7.56, 7.58, 7.6, 7.62, 7.64, 7.66, 7.68, 7.7, 7.72, 7.74, 7.76, 7.78, 7.8, 7.82, 7.84, 7.86, 7.88, 7.9, 7.92, 7.94, 7.96, 7.98, 8.0, 8.02, 8.04, 8.06, 8.08, 8.1, 8.12, 8.14, 8.16, 8.18, 8.2, 8.22, 8.24, 8.26, 8.28, 8.3, 8.32, 8.34, 8.36, 8.38, 8.4, 8.42, 8.44, 8.46, 8.48, 8.5, 8.52, 8.54, 8.56, 8.58, 8.6, 8.62, 8.64, 8.66, 8.68, 8.7, 8.72, 8.74, 8.76, 8.78, 8.8, 8.82, 8.84, 8.86, 8.88, 8.9, 8.92, 8.94, 8.96, 8.98, 9.0, 9.02, 9.04, 9.06, 9.08, 9.1, 9.12, 9.14, 9.16, 9.18, 9.2, 9.22, 9.24, 9.26, 9.28, 9.3, 9.32, 9.34, 9.36, 9.38, 9.4, 9.42, 9.44, 9.46, 9.48, 9.5, 9.52, 9.54, 9.56, 9.58, 9.6, 9.62, 9.64, 9.66, 9.68, 9.7, 9.72, 9.74, 9.76, 9.78, 9.8, 9.82, 9.84, 9.86, 9.88, 9.9, 9.92, 9.94, 9.96, 9.98, or about 10 total wt %); b) a skin conditioner (e.g., about 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2.0, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3, 2.31, 2.32, 2.33, 2.34, 2.35, 2.36, 2.37, 2.38, 2.39, 2.4, 2.41, 2.42, 2.43, 2.44, 2.45, 2.46, 2.47, 2.48, 2.49, 2.5, 2.51, 2.52, 2.53, 2.54, 2.55, 2.56, 2.57, 2.58, 2.59, 2.6, 2.61, 2.62, 2.63, 2.64, 2.65, 2.66, 2.67, 2.68, 2.69, 2.7, 2.71, 2.72, 2.73, 2.74, 2.75, 2.76, 2.77, 2.78, 2.79, 2.8, 2.81, 2.82, 2.83, 2.84, 2.85, 2.86, 2.87, 2.88, 2.89, 2.9, 2.91, 2.92, 2.93, 2.94, 2.95, 2.96, 2.97, 2.98, 2.99, or about 3.0 wt %); c) a chelating agent (e.g., about 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or about 1.0 wt %); d) humectant (e.g., about 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0, 25.0, 26.0, 27.0, 28.0, 29.0 or about 30.0 wt %); e) glyceryl stearate PEG-100 emulsifier (e.g., about 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2.0, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3, 2.31, 2.32, 2.33, 2.34, 2.35, 2.36, 2.37, 2.38, 2.39, 2.4, 2.41, 2.42, 2.43, 2.44, 2.45, 2.46, 2.47, 2.48, 2.49, 2.5, 2.51, 2.52, 2.53, 2.54, 2.55, 2.56, 2.57, 2.58, 2.59, 2.6, 2.61, 2.62, 2.63, 2.64, 2.65, 2.66, 2.67, 2.68, 2.69, 2.7, 2.71, 2.72, 2.73, 2.74, 2.75, 2.76, 2.77, 2.78, 2.79, 2.8, 2.81, 2.82, 2.83, 2.84, 2.85, 2.86, 2.87, 2.88, 2.89, 2.9, 2.91, 2.92, 2.93, 2.94, 2.95, 2.96, 2.97, 2.98, 2.99, or about 3.0 wt %); f) a mixture of emollients consisting of petrolatum and squalane (e.g., about 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24.0, 24.5, 25.0, 25.5, 26.0, 26.5, 27.0, 27.5, 28.0, 28.5, 29.0, 29.5, or about 30.0 wt %); g) a mixture of preservatives consisting of phenoxyethanol and sorbic acid (e.g., about 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, or about 1.5 total wt %); h) citric acid (e.g., about 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.55, 0.6, 0.65, 0.7, 0.75, 0.8, 0.9, 1, 2, 3, 4, 5, 6, 7, 8, 9, 10, 11, 12, 13, 14, 15, 16, 17, 18, 19, 20, 21, 2, 23, 24, 25, 26, 27, 28, 29, 30, 31, 32, 33, 34, 35, 36, 37, 38, 39, 40, 41, 42, 43, 44, 45, 46, 47, 48, 49 or about 50 wt %); i) a mixture of encapsulation aids consisting of corn syrup solids and euphorbia cerifera (candelilla) wax (e.g., about 7.0, 7.02, 7.04, 7.06, 7.08, 7.1, 7.12, 7.14, 7.16, 7.18, 7.2, 7.22, 7.24, 7.26, 7.28, 7.3, 7.32, 7.34, 7.36, 7.38, 7.4, 7.42, 7.44, 7.46, 7.48, 7.5, 7.52, 7.54, 7.56, 7.58, 7.6, 7.62, 7.64, 7.66, 7.68, 7.7, 7.72, 7.74, 7.76, 7.78, 7.8, 7.82, 7.84, 7.86, 7.88, 7.9, 7.92, 7.94, 7.96, 7.98, 8.0, 8.02, 8.04, 8.06, 8.08, 8.1, 8.12, 8.14, 8.16, 8.18, 8.2, 8.22, 8.24, 8.26, 8.28, 8.3, 8.32, 8.34, 8.36, 8.38, 8.4, 8.42, 8.44, 8.46, 8.48, 8.5, 8.52, 8.54, 8.56, 8.58, 8.6, 8.62, 8.64, 8.66, 8.68, 8.7, 8.72, 8.74, 8.76, 8.78, 8.8, 8.82, 8.84, 8.86, 8.88, 8.9, 8.92, 8.94, 8.96, 8.98, 9.0, 9.02, 9.04, 9.06, 9.08, 9.1, 9.12, 9.14, 9.16, 9.18, 9.2, 9.22, 9.24, 9.26, 9.28, 9.3, 9.32, 9.34, 9.36, 9.38, 9.4, 9.42, 9.44, 9.46, 9.48, 9.5, 9.52, 9.54, 9.56, 9.58, 9.6, 9.62, 9.64, 9.66, 9.68, 9.7, 9.72, 9.74, 9.76, 9.78, 9.8, 9.82, 9.84, 9.86, 9.88, 9.9, 9.92, 9.94, 9.96, 9.98, or about 10.0 total wt %); j) an aqueous phase thickener (e.g., about 0.1, 0.12, 0.14, 0.16, 0.18, 0.2, 0.22, 0.24, 0.26, 0.28, 0.3, 0.32, 0.34, 0.36, 0.38, 0.4, 0.42, 0.44, 0.46, 0.48, 0.5, 0.52, 0.54, 0.56, 0.58, 0.6, 0.62, 0.64, 0.66, 0.68, 0.7, 0.72, 0.74, 0.76, 0.78, 0.8, 0.82, 0.84, 0.86, 0.88, 0.9, 0.92, 0.94, 0.96, 0.98, 1.0, 1.02, 1.04, 1.06, 1.08, 1.1, 1.12, 1.14, 1.16, 1.18, 1.2, 1.22, 1.24, 1.26, 1.28, 1.3, 1.32, 1.34, 1.36, 1.38, 1.4, 1.42, 1.44, 1.46, 1.48, 1.5, 1.52, 1.54, 1.56, 1.58, 1.6, 1.62, 1.64, 1.66, 1.68, 1.7, 1.72, 1.74, 1.76, 1.78, 1.8, 1.82, 1.84, 1.86, 1.88, 1.9, 1.92, 1.94, 1.96, 1.98, or about 2.0 wt %); k) hesperidin (e.g., about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or about 3.0 wt %); and l) water.

In embodiments is provided a topical composition including: a) a mixture of barrier lipids consisting of cholesterol, one or more ceramides, and one or more free fatty acids (e.g., about 1.0, 1.1, 1.2, 1.3, 1.4, 1.5, 1.6, 1.7, 1.8, 1.9, 2.0, 2.02, 2.04, 2.06, 2.08, 2.1, 2.12, 2.14, 2.16, 2.18, 2.2, 2.22, 2.24, 2.26, 2.28, 2.3, 2.32, 2.34, 2.36, 2.38, 2.4, 2.42, 2.44, 2.46, 2.48, 2.5, 2.52, 2.54, 2.56, 2.58, 2.6, 2.62, 2.64, 2.66, 2.68, 2.7, 2.72, 2.74, 2.76, 2.78, 2.8, 2.82, 2.84, 2.86, 2.88, 2.9, 2.92, 2.94, 2.96, 2.98, 3.0, 3.02, 3.04, 3.06, 3.08, 3.1, 3.12, 3.14, 3.16, 3.18, 3.2, 3.22, 3.24, 3.26, 3.28, 3.3, 3.32, 3.34, 3.36, 3.38, 3.4, 3.42, 3.44, 3.46, 3.48, 3.5, 3.52, 3.54, 3.56, 3.58, 3.6, 3.62, 3.64, 3.66, 3.68, 3.7, 3.72, 3.74, 3.76, 3.78, 3.8, 3.82, 3.84, 3.86, 3.88, 3.9, 3.92, 3.94, 3.96, 3.98, 4.0, 4.02, 4.04, 4.06, 4.08, 4.1, 4.12, 4.14, 4.16, 4.18, 4.2, 4.22, 4.24, 4.26, 4.28, 4.3, 4.32, 4.34, 4.36, 4.38, 4.4, 4.42, 4.44, 4.46, 4.48, 4.5, 4.52, 4.54, 4.56, 4.58, 4.6, 4.62, 4.64, 4.66, 4.68, 4.7, 4.72, 4.74, 4.76, 4.78, 4.8, 4.82, 4.84, 4.86, 4.88, 4.9, 4.92, 4.94, 4.96, 4.98, 5.0, 5.02, 5.04, 5.06, 5.08, 5.1, 5.12, 5.14, 5.16, 5.18, 5.2, 5.22, 5.24, 5.26, 5.28, 5.3, 5.32, 5.34, 5.36, 5.38, 5.4, 5.42, 5.44, 5.46, 5.48, 5.5, 5.52, 5.54, 5.56, 5.58, 5.6, 5.62, 5.64, 5.66, 5.68, 5.7, 5.72, 5.74, 5.76, 5.78, 5.8, 5.82, 5.84, 5.86, 5.88, 5.9, 5.92, 5.94, 5.96, 5.98, 6.0, 6.02, 6.04, 6.06, 6.08, 6.1, 6.12, 6.14, 6.16, 6.18, 6.2, 6.22, 6.24, 6.26, 6.28, 6.3, 6.32, 6.34, 6.36, 6.38, 6.4, 6.42, 6.44, 6.46, 6.48, 6.5, 6.52, 6.54, 6.56, 6.58, 6.6, 6.62, 6.64, 6.66, 6.68, 6.7, 6.72, 6.74, 6.76, 6.78, 6.8, 6.82, 6.84, 6.86, 6.88, 6.9, 6.92, 6.94, 6.96, 6.98, 7.0, 7.02, 7.04, 7.06, 7.08, 7.1, 7.12, 7.14, 7.16, 7.18, 7.2, 7.22, 7.24, 7.26, 7.28, 7.3, 7.32, 7.34, 7.36, 7.38, 7.4, 7.42, 7.44, 7.46, 7.48, 7.5, 7.52, 7.54, 7.56, 7.58, 7.6, 7.62, 7.64, 7.66, 7.68, 7.7, 7.72, 7.74, 7.76, 7.78, 7.8, 7.82, 7.84, 7.86, 7.88, 7.9, 7.92, 7.94, 7.96, 7.98, 8.0, 8.02, 8.04, 8.06, 8.08, 8.1, 8.12, 8.14, 8.16, 8.18, 8.2, 8.22, 8.24, 8.26, 8.28, 8.3, 8.32, 8.34, 8.36, 8.38, 8.4, 8.42, 8.44, 8.46, 8.48, 8.5, 8.52, 8.54, 8.56, 8.58, 8.6, 8.62, 8.64, 8.66, 8.68, 8.7, 8.72, 8.74, 8.76, 8.78, 8.8, 8.82, 8.84, 8.86, 8.88, 8.9, 8.92, 8.94, 8.96, 8.98, 9.0, 9.02, 9.04, 9.06, 9.08, 9.1, 9.12, 9.14, 9.16, 9.18, 9.2, 9.22, 9.24, 9.26, 9.28, 9.3, 9.32, 9.34, 9.36, 9.38, 9.4, 9.42, 9.44, 9.46, 9.48, 9.5, 9.52, 9.54, 9.56, 9.58, 9.6, 9.62, 9.64, 9.66, 9.68, 9.7, 9.72, 9.74, 9.76, 9.78, 9.8, 9.82, 9.84, 9.86, 9.88, 9.9, 9.92, 9.94, 9.96, 9.98, or about 10 total wt %); b) a skin conditioner (e.g., about 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2.0, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3, 2.31, 2.32, 2.33, 2.34, 2.35, 2.36, 2.37, 2.38, 2.39, 2.4, 2.41, 2.42, 2.43, 2.44, 2.45, 2.46, 2.47, 2.48, 2.49, 2.5, 2.51, 2.52, 2.53, 2.54, 2.55, 2.56, 2.57, 2.58, 2.59, 2.6, 2.61, 2.62, 2.63, 2.64, 2.65, 2.66, 2.67, 2.68, 2.69, 2.7, 2.71, 2.72, 2.73, 2.74, 2.75, 2.76, 2.77, 2.78, 2.79, 2.8, 2.81, 2.82, 2.83, 2.84, 2.85, 2.86, 2.87, 2.88, 2.89, 2.9, 2.91, 2.92, 2.93, 2.94, 2.95, 2.96, 2.97, 2.98, 2.99, or about 3.0 wt %); c) a chelating agent (e.g., about 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or about 1.0 wt %); d) humectant (e.g., about 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0, 25.0, 26.0, 27.0, 28.0, 29.0 or about 30.0 wt %); e) glyceryl stearate PEG-100 emulsifier (e.g., about 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2.0, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3, 2.31, 2.32, 2.33, 2.34, 2.35, 2.36, 2.37, 2.38, 2.39, 2.4, 2.41, 2.42, 2.43, 2.44, 2.45, 2.46, 2.47, 2.48, 2.49, 2.5, 2.51, 2.52, 2.53, 2.54, 2.55, 2.56, 2.57, 2.58, 2.59, 2.6, 2.61, 2.62, 2.63, 2.64, 2.65, 2.66, 2.67, 2.68, 2.69, 2.7, 2.71, 2.72, 2.73, 2.74, 2.75, 2.76, 2.77, 2.78, 2.79, 2.8, 2.81, 2.82, 2.83, 2.84, 2.85, 2.86, 2.87, 2.88, 2.89, 2.9, 2.91, 2.92, 2.93, 2.94, 2.95, 2.96, 2.97, 2.98, 2.99, or about 3.0 wt %); f) a mixture of emollients consisting of petrolatum and squalane (e.g., about 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24.0, 24.5, 25.0, 25.5, 26.0, 26.5, 27.0, 27.5, 28.0, 28.5, 29.0, 29.5, or about 30.0 wt %); g) a mixture of preservatives consisting of phenoxyethanol and sorbic acid (e.g., about 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, or about 1.5 total wt %); h) citric acid (e.g., about 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.55, 0.6, 0.65, 0.7, 0.75, 0.8, 0.9, 1, 2, 3, 4, 5, 6, 7, 8, 9, 10, 11, 12, 13, 14, 15, 16, 17, 18, 19, 20, 21, 2, 23, 24, 25, 26, 27, 28, 29, 30, 31, 32, 33, 34, 35, 36, 37, 38, 39, 40, 41, 42, 43, 44, 45, 46, 47, 48, 49 or about 50 wt %); i) a mixture of encapsulation aids consisting of corn syrup solids and euphorbia cerifera (candelilla) wax (e.g., about 7.0, 7.02, 7.04, 7.06, 7.08, 7.1, 7.12, 7.14, 7.16, 7.18, 7.2, 7.22, 7.24, 7.26, 7.28, 7.3, 7.32, 7.34, 7.36, 7.38, 7.4, 7.42, 7.44, 7.46, 7.48, 7.5, 7.52, 7.54, 7.56, 7.58, 7.6, 7.62, 7.64, 7.66, 7.68, 7.7, 7.72, 7.74, 7.76, 7.78, 7.8, 7.82, 7.84, 7.86, 7.88, 7.9, 7.92, 7.94, 7.96, 7.98, 8.0, 8.02, 8.04, 8.06, 8.08, 8.1, 8.12, 8.14, 8.16, 8.18, 8.2, 8.22, 8.24, 8.26, 8.28, 8.3, 8.32, 8.34, 8.36, 8.38, 8.4, 8.42, 8.44, 8.46, 8.48, 8.5, 8.52, 8.54, 8.56, 8.58, 8.6, 8.62, 8.64, 8.66, 8.68, 8.7, 8.72, 8.74, 8.76, 8.78, 8.8, 8.82, 8.84, 8.86, 8.88, 8.9, 8.92, 8.94, 8.96, 8.98, 9.0, 9.02, 9.04, 9.06, 9.08, 9.1, 9.12, 9.14, 9.16, 9.18, 9.2, 9.22, 9.24, 9.26, 9.28, 9.3, 9.32, 9.34, 9.36, 9.38, 9.4, 9.42, 9.44, 9.46, 9.48, 9.5, 9.52, 9.54, 9.56, 9.58, 9.6, 9.62, 9.64, 9.66, 9.68, 9.7, 9.72, 9.74, 9.76, 9.78, 9.8, 9.82, 9.84, 9.86, 9.88, 9.9, 9.92, 9.94, 9.96, 9.98, or about 10.0 total wt %); j) an aqueous phase thickener (e.g., about 0.1, 0.12, 0.14, 0.16, 0.18, 0.2, 0.22, 0.24, 0.26, 0.28, 0.3, 0.32, 0.34, 0.36, 0.38, 0.4, 0.42, 0.44, 0.46, 0.48, 0.5, 0.52, 0.54, 0.56, 0.58, 0.6, 0.62, 0.64, 0.66, 0.68, 0.7, 0.72, 0.74, 0.76, 0.78, 0.8, 0.82, 0.84, 0.86, 0.88, 0.9, 0.92, 0.94, 0.96, 0.98, 1.0, 1.02, 1.04, 1.06, 1.08, 1.1, 1.12, 1.14, 1.16, 1.18, 1.2, 1.22, 1.24, 1.26, 1.28, 1.3, 1.32, 1.34, 1.36, 1.38, 1.4, 1.42, 1.44, 1.46, 1.48, 1.5, 1.52, 1.54, 1.56, 1.58, 1.6, 1.62, 1.64, 1.66, 1.68, 1.7, 1.72, 1.74, 1.76, 1.78, 1.8, 1.82, 1.84, 1.86, 1.88, 1.9, 1.92, 1.94, 1.96, 1.98, or about 2.0 wt %); k) hesperidin (e.g., about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or about 3.0 wt %); and l) water.

In embodiments is provided a composition including: a) a mixture of barrier lipids selected from the group consisting of cholesterol, ceramides, free fatty acids and mixtures of two or more thereof (e.g., about 1.0, 1.1, 1.2, 1.3, 1.4, 1.5, 1.6, 1.7, 1.8, 1.9, 2.0, 2.02, 2.04, 2.06, 2.08, 2.1, 2.12, 2.14, 2.16, 2.18, 2.2, 2.22, 2.24, 2.26, 2.28, 2.3, 2.32, 2.34, 2.36, 2.38, 2.4, 2.42, 2.44, 2.46, 2.48, 2.5, 2.52, 2.54, 2.56, 2.58, 2.6, 2.62, 2.64, 2.66, 2.68, 2.7, 2.72, 2.74, 2.76, 2.78, 2.8, 2.82, 2.84, 2.86, 2.88, 2.9, 2.92, 2.94, 2.96, 2.98, 3.0, 3.02, 3.04, 3.06, 3.08, 3.1, 3.12, 3.14, 3.16, 3.18, 3.2, 3.22, 3.24, 3.26, 3.28, 3.3, 3.32, 3.34, 3.36, 3.38, 3.4, 3.42, 3.44, 3.46, 3.48, 3.5, 3.52, 3.54, 3.56, 3.58, 3.6, 3.62, 3.64, 3.66, 3.68, 3.7, 3.72, 3.74, 3.76, 3.78, 3.8, 3.82, 3.84, 3.86, 3.88, 3.9, 3.92, 3.94, 3.96, 3.98, 4.0, 4.02, 4.04, 4.06, 4.08, 4.1, 4.12, 4.14, 4.16, 4.18, 4.2, 4.22, 4.24, 4.26, 4.28, 4.3, 4.32, 4.34, 4.36, 4.38, 4.4, 4.42, 4.44, 4.46, 4.48, 4.5, 4.52, 4.54, 4.56, 4.58, 4.6, 4.62, 4.64, 4.66, 4.68, 4.7, 4.72, 4.74, 4.76, 4.78, 4.8, 4.82, 4.84, 4.86, 4.88, 4.9, 4.92, 4.94, 4.96, 4.98, 5.0, 5.02, 5.04, 5.06, 5.08, 5.1, 5.12, 5.14, 5.16, 5.18, 5.2, 5.22, 5.24, 5.26, 5.28, 5.3, 5.32, 5.34, 5.36, 5.38, 5.4, 5.42, 5.44, 5.46, 5.48, 5.5, 5.52, 5.54, 5.56, 5.58, 5.6, 5.62, 5.64, 5.66, 5.68, 5.7, 5.72, 5.74, 5.76, 5.78, 5.8, 5.82, 5.84, 5.86, 5.88, 5.9, 5.92, 5.94, 5.96, 5.98, 6.0, 6.02, 6.04, 6.06, 6.08, 6.1, 6.12, 6.14, 6.16, 6.18, 6.2, 6.22, 6.24, 6.26, 6.28, 6.3, 6.32, 6.34, 6.36, 6.38, 6.4, 6.42, 6.44, 6.46, 6.48, 6.5, 6.52, 6.54, 6.56, 6.58, 6.6, 6.62, 6.64, 6.66, 6.68, 6.7, 6.72, 6.74, 6.76, 6.78, 6.8, 6.82, 6.84, 6.86, 6.88, 6.9, 6.92, 6.94, 6.96, 6.98, 7.0, 7.02, 7.04, 7.06, 7.08, 7.1, 7.12, 7.14, 7.16, 7.18, 7.2, 7.22, 7.24, 7.26, 7.28, 7.3, 7.32, 7.34, 7.36, 7.38, 7.4, 7.42, 7.44, 7.46, 7.48, 7.5, 7.52, 7.54, 7.56, 7.58, 7.6, 7.62, 7.64, 7.66, 7.68, 7.7, 7.72, 7.74, 7.76, 7.78, 7.8, 7.82, 7.84, 7.86, 7.88, 7.9, 7.92, 7.94, 7.96, 7.98, 8.0, 8.02, 8.04, 8.06, 8.08, 8.1, 8.12, 8.14, 8.16, 8.18, 8.2, 8.22, 8.24, 8.26, 8.28, 8.3, 8.32, 8.34, 8.36, 8.38, 8.4, 8.42, 8.44, 8.46, 8.48, 8.5, 8.52, 8.54, 8.56, 8.58, 8.6, 8.62, 8.64, 8.66, 8.68, 8.7, 8.72, 8.74, 8.76, 8.78, 8.8, 8.82, 8.84, 8.86, 8.88, 8.9, 8.92, 8.94, 8.96, 8.98, 9.0, 9.02, 9.04, 9.06, 9.08, 9.1, 9.12, 9.14, 9.16, 9.18, 9.2, 9.22, 9.24, 9.26, 9.28, 9.3, 9.32, 9.34, 9.36, 9.38, 9.4, 9.42, 9.44, 9.46, 9.48, 9.5, 9.52, 9.54, 9.56, 9.58, 9.6, 9.62, 9.64, 9.66, 9.68, 9.7, 9.72, 9.74, 9.76, 9.78, 9.8, 9.82, 9.84, 9.86, 9.88, 9.9, 9.92, 9.94, 9.96, 9.98, or about 10 total wt %) b) hesperidin (e.g., about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or about 3.0 wt %); c) an acid buffer (e.g., about 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.55, 0.6, 0.65, 0.7, 0.75, 0.8, 0.9, 1, 2, 3, 4, 5, 6, 7, 8, 9, 10, 11, 12, 13, 14, 15, 16, 17, 18, 19, 20, 21, 2, 23, 24, 25, 26, 27, 28, 29, 30, 31, 32, 33, 34, 35, 36, 37, 38, 39, 40, 41, 42, 43, 44, 45, 46, 47, 48, 49 or about 50 wt %); and d) at least one glucocorticoid (e.g., about 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2.0, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3, 2.31, 2.32, 2.33, 2.34, 2.35, 2.36, 2.37, 2.38, 2.39, 2.4, 2.41, 2.42, 2.43, 2.44, 2.45, 2.46, 2.47, 2.48, 2.49, 2.5, 2.51, 2.52, 2.53, 2.54, 2.55, 2.56, 2.57, 2.58, 2.59, 2.6, 2.61, 2.62, 2.63, 2.64, 2.65, 2.66, 2.67, 2.68, 2.69, 2.7, 2.71, 2.72, 2.73, 2.74, 2.75, 2.76, 2.77, 2.78, 2.79, 2.8, 2.81, 2.82, 2.83, 2.84, 2.85, 2.86, 2.87, 2.88, 2.89, 2.9, 2.91, 2.92, 2.93, 2.94, 2.95, 2.96, 2.97, 2.98, 2.99, or about 3.0 wt %).

In embodiments, the composition further includes an acid buffer. In embodiments, the pH of the composition is from 3 to 5.5. In embodiments, the composition includes 1 to 50 wt % of total cholesterol. In embodiments, the composition includes 0.1 to 10 wt % of total cholesterol. In embodiments, the composition includes 0.1 to 5 wt % of total cholesterol. In embodiments, the composition includes 0.001 to 50 wt % of hesperidin. In embodiments, the composition includes 0.1 to 10 wt % of hesperidin. In embodiments, the composition includes 0.1 to 5 wt % of hesperidin. In embodiments, the composition includes 0.1 to 10 wt % total ceramide. In embodiments, the composition includes 0.1 to 5 wt % total ceramide. In embodiments, the composition includes 0.01 to 6 wt % total free fatty acid. In embodiments, the composition includes 0.1 to 2 wt % total free fatty acid. In embodiments, the composition includes 0.05 to 5 wt % of the acid buffer. In embodiments, the composition includes 0.1 to 5 wt % of the acid buffer.

In embodiments, the composition further includes a skin conditioner. In embodiments, the composition includes 0.1 to 10 wt % of the skin conditioner. In embodiments, the composition includes 0.1 to 5 wt % of the skin conditioner. In embodiments, the skin conditioner is dimethicone. In embodiments, the composition further includes a chelating agent. In embodiments, the composition includes 0.001 to 5 wt % of the chelating agent. In embodiments, the composition includes 0.01 to 1 wt % of the chelating agent. In embodiments, the chelating agent is disodium EDTA. In embodiments, the composition further includes a humectant. In embodiments, the composition includes 1 to 30 wt % of the humectant. In embodiments, the composition includes 5 to 30 wt % of the humectant. In embodiments, the humectant is glycerin. In embodiments, the composition further includes an emulsifier. In embodiments, the composition includes 0.5 to 20 wt % of the emulsifier. In embodiments, the composition includes 0.2 to 10 wt % of the emulsifier. In embodiments, the emulsifier is glyceryl stearate PEG-100. In embodiments, the composition further includes an emollient. In embodiments, the composition includes 1 to 30 wt % of the emollient. In embodiments, the composition includes 0.2 to 10 wt % of the emollient. In embodiments, the emollient is petrolatum. In embodiments, the emollient is squalane. In embodiments, the composition further includes a preservative. In embodiments, the composition includes 0.1 to 10 wt % of the preservative. In embodiments, the composition includes 0.01 to 5 wt % of the preservative. In embodiments, the preservative is phenoxyethanol. In embodiments, the preservative is sorbic acid. In embodiments, the composition further includes an encapsulation aid. In embodiments, the composition includes 1 to 25 wt % of the encapsulation aid. In embodiments, the composition includes 0.01 to 30 wt % of the encapsulation aid. In embodiments, the encapsulation aid is euphorbia cerifera (candelilla) wax. In embodiments, the encapsulation aid is corn syrup solids. In embodiments, the composition further includes an aqueous phase thickener. In embodiments, the composition includes 0.01 to 3 wt % of the aqueous phase thickener. In embodiments, the aqueous phase thickener is xanthan gum. In embodiments, the ceramide is hydroxypropyl bispalmitamide MEA (Ceramide PC-104). In embodiments, the free fatty acid is conjugated linoleic acid (CLA). In embodiments, the composition further includes a glucocorticoid. In embodiments, the composition includes 0.001 to 5 wt % of the glucocorticoid. In embodiments, the composition is a topical composition.

In embodiments is provided a topical composition, the composition including: a) cholesterol (e.g., 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 wt %); b) an acid buffer (e.g., 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.55, 0.6, 0.65, 0.7, 0.75, 0.8, 0.9, 1, 2, 3, 4, 5, 6, 7, 8, 9, 10, 11, 12, 13, 14, 15, 16, 17, 18, 19, 20, 21, 2, 23, 24, 25, 26, 27, 28, 29, 30, 31, 32, 33, 34, 35, 36, 37, 38, 39, 40, 41, 42, 43, 44, 45, 46, 47, 48, 49 or 50 wt %); and c) hesperidin (e.g., 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 wt %); wherein the pH of the topical composition is 3 to 5.5.

In embodiments is provided a topical composition, the composition including: a) a mixture of barrier lipids selected from the group consisting of cholesterol, at least one ceramide, at least one free fatty acid, and mixtures of two or more thereof (e.g., 1.0, 1.1, 1.2, 1.3, 1.4, 1.5, 1.6, 1.7, 1.8, 1.9, 2.0, 2.02, 2.04, 2.06, 2.08, 2.1, 2.12, 2.14, 2.16, 2.18, 2.2, 2.22, 2.24, 2.26, 2.28, 2.3, 2.32, 2.34, 2.36, 2.38, 2.4, 2.42, 2.44, 2.46, 2.48, 2.5, 2.52, 2.54, 2.56, 2.58, 2.6, 2.62, 2.64, 2.66, 2.68, 2.7, 2.72, 2.74, 2.76, 2.78, 2.8, 2.82, 2.84, 2.86, 2.88, 2.9, 2.92, 2.94, 2.96, 2.98, 3.0, 3.02, 3.04, 3.06, 3.08, 3.1, 3.12, 3.14, 3.16, 3.18, 3.2, 3.22, 3.24, 3.26, 3.28, 3.3, 3.32, 3.34, 3.36, 3.38, 3.4, 3.42, 3.44, 3.46, 3.48, 3.5, 3.52, 3.54, 3.56, 3.58, 3.6, 3.62, 3.64, 3.66, 3.68, 3.7, 3.72, 3.74, 3.76, 3.78, 3.8, 3.82, 3.84, 3.86, 3.88, 3.9, 3.92, 3.94, 3.96, 3.98, 4.0, 4.02, 4.04, 4.06, 4.08, 4.1, 4.12, 4.14, 4.16, 4.18, 4.2, 4.22, 4.24, 4.26, 4.28, 4.3, 4.32, 4.34, 4.36, 4.38, 4.4, 4.42, 4.44, 4.46, 4.48, 4.5, 4.52, 4.54, 4.56, 4.58, 4.6, 4.62, 4.64, 4.66, 4.68, 4.7, 4.72, 4.74, 4.76, 4.78, 4.8, 4.82, 4.84, 4.86, 4.88, 4.9, 4.92, 4.94, 4.96, 4.98, 5.0, 5.02, 5.04, 5.06, 5.08, 5.1, 5.12, 5.14, 5.16, 5.18, 5.2, 5.22, 5.24, 5.26, 5.28, 5.3, 5.32, 5.34, 5.36, 5.38, 5.4, 5.42, 5.44, 5.46, 5.48, 5.5, 5.52, 5.54, 5.56, 5.58, 5.6, 5.62, 5.64, 5.66, 5.68, 5.7, 5.72, 5.74, 5.76, 5.78, 5.8, 5.82, 5.84, 5.86, 5.88, 5.9, 5.92, 5.94, 5.96, 5.98, 6.0, 6.02, 6.04, 6.06, 6.08, 6.1, 6.12, 6.14, 6.16, 6.18, 6.2, 6.22, 6.24, 6.26, 6.28, 6.3, 6.32, 6.34, 6.36, 6.38, 6.4, 6.42, 6.44, 6.46, 6.48, 6.5, 6.52, 6.54, 6.56, 6.58, 6.6, 6.62, 6.64, 6.66, 6.68, 6.7, 6.72, 6.74, 6.76, 6.78, 6.8, 6.82, 6.84, 6.86, 6.88, 6.9, 6.92, 6.94, 6.96, 6.98, 7.0, 7.02, 7.04, 7.06, 7.08, 7.1, 7.12, 7.14, 7.16, 7.18, 7.2, 7.22, 7.24, 7.26, 7.28, 7.3, 7.32, 7.34, 7.36, 7.38, 7.4, 7.42, 7.44, 7.46, 7.48, 7.5, 7.52, 7.54, 7.56, 7.58, 7.6, 7.62, 7.64, 7.66, 7.68, 7.7, 7.72, 7.74, 7.76, 7.78, 7.8, 7.82, 7.84, 7.86, 7.88, 7.9, 7.92, 7.94, 7.96, 7.98, 8.0, 8.02, 8.04, 8.06, 8.08, 8.1, 8.12, 8.14, 8.16, 8.18, 8.2, 8.22, 8.24, 8.26, 8.28, 8.3, 8.32, 8.34, 8.36, 8.38, 8.4, 8.42, 8.44, 8.46, 8.48, 8.5, 8.52, 8.54, 8.56, 8.58, 8.6, 8.62, 8.64, 8.66, 8.68, 8.7, 8.72, 8.74, 8.76, 8.78, 8.8, 8.82, 8.84, 8.86, 8.88, 8.9, 8.92, 8.94, 8.96, 8.98, 9.0, 9.02, 9.04, 9.06, 9.08, 9.1, 9.12, 9.14, 9.16, 9.18, 9.2, 9.22, 9.24, 9.26, 9.28, 9.3, 9.32, 9.34, 9.36, 9.38, 9.4, 9.42, 9.44, 9.46, 9.48, 9.5, 9.52, 9.54, 9.56, 9.58, 9.6, 9.62, 9.64, 9.66, 9.68, 9.7, 9.72, 9.74, 9.76, 9.78, 9.8, 9.82, 9.84, 9.86, 9.88, 9.9, 9.92, 9.94, 9.96, 9.98, or 10 total wt %); b) a skin conditioner (e.g., 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2.0, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3, 2.31, 2.32, 2.33, 2.34, 2.35, 2.36, 2.37, 2.38, 2.39, 2.4, 2.41, 2.42, 2.43, 2.44, 2.45, 2.46, 2.47, 2.48, 2.49, 2.5, 2.51, 2.52, 2.53, 2.54, 2.55, 2.56, 2.57, 2.58, 2.59, 2.6, 2.61, 2.62, 2.63, 2.64, 2.65, 2.66, 2.67, 2.68, 2.69, 2.7, 2.71, 2.72, 2.73, 2.74, 2.75, 2.76, 2.77, 2.78, 2.79, 2.8, 2.81, 2.82, 2.83, 2.84, 2.85, 2.86, 2.87, 2.88, 2.89, 2.9, 2.91, 2.92, 2.93, 2.94, 2.95, 2.96, 2.97, 2.98, 2.99, or 3.0 wt %); c) a chelating agent (e.g., 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1.0 wt %); d) a humectant (e.g., 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0, 25.0, 26.0, 27.0, 28.0, 29.0 or 30.0 wt %); e) an emulsifier (1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2.0, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3, 2.31, 2.32, 2.33, 2.34, 2.35, 2.36, 2.37, 2.38, 2.39, 2.4, 2.41, 2.42, 2.43, 2.44, 2.45, 2.46, 2.47, 2.48, 2.49, 2.5, 2.51, 2.52, 2.53, 2.54, 2.55, 2.56, 2.57, 2.58, 2.59, 2.6, 2.61, 2.62, 2.63, 2.64, 2.65, 2.66, 2.67, 2.68, 2.69, 2.7, 2.71, 2.72, 2.73, 2.74, 2.75, 2.76, 2.77, 2.78, 2.79, 2.8, 2.81, 2.82, 2.83, 2.84, 2.85, 2.86, 2.87, 2.88, 2.89, 2.9, 2.91, 2.92, 2.93, 2.94, 2.95, 2.96, 2.97, 2.98, 2.99, or 3.0 wt %); f) one or more emollients (e.g., 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24.0, 24.5, 25.0, 25.5, 26.0, 26.5, 27.0, 27.5, 28.0, 28.5, 29.0, 29.5, or 30.0 wt %); g) one or more preservatives (e.g., 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, or total 1.5 wt %); h) an acid buffer (e.g., 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.55, 0.6, 0.65, 0.7, 0.75, 0.8, 0.9, 1, 2, 3, 4, 5, 6, 7, 8, 9, 10, 11, 12, 13, 14, 15, 16, 17, 18, 19, 20, 21, 2, 23, 24, 25, 26, 27, 28, 29, 30, 31, 32, 33, 34, 35, 36, 37, 38, 39, 40, 41, 42, 43, 44, 45, 46, 47, 48, 49 or 50 wt %); i) one or more encapsulation aids (e.g., 7.0, 7.02, 7.04, 7.06, 7.08, 7.1, 7.12, 7.14, 7.16, 7.18, 7.2, 7.22, 7.24, 7.26, 7.28, 7.3, 7.32, 7.34, 7.36, 7.38, 7.4, 7.42, 7.44, 7.46, 7.48, 7.5, 7.52, 7.54, 7.56, 7.58, 7.6, 7.62, 7.64, 7.66, 7.68, 7.7, 7.72, 7.74, 7.76, 7.78, 7.8, 7.82, 7.84, 7.86, 7.88, 7.9, 7.92, 7.94, 7.96, 7.98, 8.0, 8.02, 8.04, 8.06, 8.08, 8.1, 8.12, 8.14, 8.16, 8.18, 8.2, 8.22, 8.24, 8.26, 8.28, 8.3, 8.32, 8.34, 8.36, 8.38, 8.4, 8.42, 8.44, 8.46, 8.48, 8.5, 8.52, 8.54, 8.56, 8.58, 8.6, 8.62, 8.64, 8.66, 8.68, 8.7, 8.72, 8.74, 8.76, 8.78, 8.8, 8.82, 8.84, 8.86, 8.88, 8.9, 8.92, 8.94, 8.96, 8.98, 9.0, 9.02, 9.04, 9.06, 9.08, 9.1, 9.12, 9.14, 9.16, 9.18, 9.2, 9.22, 9.24, 9.26, 9.28, 9.3, 9.32, 9.34, 9.36, 9.38, 9.4, 9.42, 9.44, 9.46, 9.48, 9.5, 9.52, 9.54, 9.56, 9.58, 9.6, 9.62, 9.64, 9.66, 9.68, 9.7, 9.72, 9.74, 9.76, 9.78, 9.8, 9.82, 9.84, 9.86, 9.88, 9.9, 9.92, 9.94, 9.96, 9.98, or 10.0 total wt %; j) an aqueous phase thickener (e.g., 0.1, 0.12, 0.14, 0.16, 0.18, 0.2, 0.22, 0.24, 0.26, 0.28, 0.3, 0.32, 0.34, 0.36, 0.38, 0.4, 0.42, 0.44, 0.46, 0.48, 0.5, 0.52, 0.54, 0.56, 0.58, 0.6, 0.62, 0.64, 0.66, 0.68, 0.7, 0.72, 0.74, 0.76, 0.78, 0.8, 0.82, 0.84, 0.86, 0.88, 0.9, 0.92, 0.94, 0.96, 0.98, 1.0, 1.02, 1.04, 1.06, 1.08, 1.1, 1.12, 1.14, 1.16, 1.18, 1.2, 1.22, 1.24, 1.26, 1.28, 1.3, 1.32, 1.34, 1.36, 1.38, 1.4, 1.42, 1.44, 1.46, 1.48, 1.5, 1.52, 1.54, 1.56, 1.58, 1.6, 1.62, 1.64, 1.66, 1.68, 1.7, 1.72, 1.74, 1.76, 1.78, 1.8, 1.82, 1.84, 1.86, 1.88, 1.9, 1.92, 1.94, 1.96, 1.98, or 2.0 wt %); and k) hesperidin (e.g., 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 wt %).

In embodiments is provided a topical composition consisting of: a) a mixture of barrier lipids consisting of cholesterol, one or more ceramides, and one or more free fatty acids (e.g., 1.0, 1.1, 1.2, 1.3, 1.4, 1.5, 1.6, 1.7, 1.8, 1.9, 2.0, 2.02, 2.04, 2.06, 2.08, 2.1, 2.12, 2.14, 2.16, 2.18, 2.2, 2.22, 2.24, 2.26, 2.28, 2.3, 2.32, 2.34, 2.36, 2.38, 2.4, 2.42, 2.44, 2.46, 2.48, 2.5, 2.52, 2.54, 2.56, 2.58, 2.6, 2.62, 2.64, 2.66, 2.68, 2.7, 2.72, 2.74, 2.76, 2.78, 2.8, 2.82, 2.84, 2.86, 2.88, 2.9, 2.92, 2.94, 2.96, 2.98, 3.0, 3.02, 3.04, 3.06, 3.08, 3.1, 3.12, 3.14, 3.16, 3.18, 3.2, 3.22, 3.24, 3.26, 3.28, 3.3, 3.32, 3.34, 3.36, 3.38, 3.4, 3.42, 3.44, 3.46, 3.48, 3.5, 3.52, 3.54, 3.56, 3.58, 3.6, 3.62, 3.64, 3.66, 3.68, 3.7, 3.72, 3.74, 3.76, 3.78, 3.8, 3.82, 3.84, 3.86, 3.88, 3.9, 3.92, 3.94, 3.96, 3.98, 4.0, 4.02, 4.04, 4.06, 4.08, 4.1, 4.12, 4.14, 4.16, 4.18, 4.2, 4.22, 4.24, 4.26, 4.28, 4.3, 4.32, 4.34, 4.36, 4.38, 4.4, 4.42, 4.44, 4.46, 4.48, 4.5, 4.52, 4.54, 4.56, 4.58, 4.6, 4.62, 4.64, 4.66, 4.68, 4.7, 4.72, 4.74, 4.76, 4.78, 4.8, 4.82, 4.84, 4.86, 4.88, 4.9, 4.92, 4.94, 4.96, 4.98, 5.0, 5.02, 5.04, 5.06, 5.08, 5.1, 5.12, 5.14, 5.16, 5.18, 5.2, 5.22, 5.24, 5.26, 5.28, 5.3, 5.32, 5.34, 5.36, 5.38, 5.4, 5.42, 5.44, 5.46, 5.48, 5.5, 5.52, 5.54, 5.56, 5.58, 5.6, 5.62, 5.64, 5.66, 5.68, 5.7, 5.72, 5.74, 5.76, 5.78, 5.8, 5.82, 5.84, 5.86, 5.88, 5.9, 5.92, 5.94, 5.96, 5.98, 6.0, 6.02, 6.04, 6.06, 6.08, 6.1, 6.12, 6.14, 6.16, 6.18, 6.2, 6.22, 6.24, 6.26, 6.28, 6.3, 6.32, 6.34, 6.36, 6.38, 6.4, 6.42, 6.44, 6.46, 6.48, 6.5, 6.52, 6.54, 6.56, 6.58, 6.6, 6.62, 6.64, 6.66, 6.68, 6.7, 6.72, 6.74, 6.76, 6.78, 6.8, 6.82, 6.84, 6.86, 6.88, 6.9, 6.92, 6.94, 6.96, 6.98, 7.0, 7.02, 7.04, 7.06, 7.08, 7.1, 7.12, 7.14, 7.16, 7.18, 7.2, 7.22, 7.24, 7.26, 7.28, 7.3, 7.32, 7.34, 7.36, 7.38, 7.4, 7.42, 7.44, 7.46, 7.48, 7.5, 7.52, 7.54, 7.56, 7.58, 7.6, 7.62, 7.64, 7.66, 7.68, 7.7, 7.72, 7.74, 7.76, 7.78, 7.8, 7.82, 7.84, 7.86, 7.88, 7.9, 7.92, 7.94, 7.96, 7.98, 8.0, 8.02, 8.04, 8.06, 8.08, 8.1, 8.12, 8.14, 8.16, 8.18, 8.2, 8.22, 8.24, 8.26, 8.28, 8.3, 8.32, 8.34, 8.36, 8.38, 8.4, 8.42, 8.44, 8.46, 8.48, 8.5, 8.52, 8.54, 8.56, 8.58, 8.6, 8.62, 8.64, 8.66, 8.68, 8.7, 8.72, 8.74, 8.76, 8.78, 8.8, 8.82, 8.84, 8.86, 8.88, 8.9, 8.92, 8.94, 8.96, 8.98, 9.0, 9.02, 9.04, 9.06, 9.08, 9.1, 9.12, 9.14, 9.16, 9.18, 9.2, 9.22, 9.24, 9.26, 9.28, 9.3, 9.32, 9.34, 9.36, 9.38, 9.4, 9.42, 9.44, 9.46, 9.48, 9.5, 9.52, 9.54, 9.56, 9.58, 9.6, 9.62, 9.64, 9.66, 9.68, 9.7, 9.72, 9.74, 9.76, 9.78, 9.8, 9.82, 9.84, 9.86, 9.88, 9.9, 9.92, 9.94, 9.96, 9.98, or 10 total wt %); b) a skin conditioner (e.g., 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2.0, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3, 2.31, 2.32, 2.33, 2.34, 2.35, 2.36, 2.37, 2.38, 2.39, 2.4, 2.41, 2.42, 2.43, 2.44, 2.45, 2.46, 2.47, 2.48, 2.49, 2.5, 2.51, 2.52, 2.53, 2.54, 2.55, 2.56, 2.57, 2.58, 2.59, 2.6, 2.61, 2.62, 2.63, 2.64, 2.65, 2.66, 2.67, 2.68, 2.69, 2.7, 2.71, 2.72, 2.73, 2.74, 2.75, 2.76, 2.77, 2.78, 2.79, 2.8, 2.81, 2.82, 2.83, 2.84, 2.85, 2.86, 2.87, 2.88, 2.89, 2.9, 2.91, 2.92, 2.93, 2.94, 2.95, 2.96, 2.97, 2.98, 2.99, or 3.0 wt %); c) a chelating agent (e.g., 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1.0 wt %); d) humectant (e.g., 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0, 25.0, 26.0, 27.0, 28.0, 29.0 or 30.0 wt %); e) glyceryl stearate PEG-100 emulsifier (e.g., 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2.0, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3, 2.31, 2.32, 2.33, 2.34, 2.35, 2.36, 2.37, 2.38, 2.39, 2.4, 2.41, 2.42, 2.43, 2.44, 2.45, 2.46, 2.47, 2.48, 2.49, 2.5, 2.51, 2.52, 2.53, 2.54, 2.55, 2.56, 2.57, 2.58, 2.59, 2.6, 2.61, 2.62, 2.63, 2.64, 2.65, 2.66, 2.67, 2.68, 2.69, 2.7, 2.71, 2.72, 2.73, 2.74, 2.75, 2.76, 2.77, 2.78, 2.79, 2.8, 2.81, 2.82, 2.83, 2.84, 2.85, 2.86, 2.87, 2.88, 2.89, 2.9, 2.91, 2.92, 2.93, 2.94, 2.95, 2.96, 2.97, 2.98, 2.99, or 3.0 wt %); f) a mixture of emollients consisting of petrolatum and squalane (e.g., 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24.0, 24.5, 25.0, 25.5, 26.0, 26.5, 27.0, 27.5, 28.0, 28.5, 29.0, 29.5, or 30.0 wt %); g) a mixture of preservatives consisting of phenoxyethanol and sorbic acid (e.g., 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, or 1.5 total wt %); h) citric acid (e.g., 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.55, 0.6, 0.65, 0.7, 0.75, 0.8, 0.9, 1, 2, 3, 4, 5, 6, 7, 8, 9, 10, 11, 12, 13, 14, 15, 16, 17, 18, 19, 20, 21, 2, 23, 24, 25, 26, 27, 28, 29, 30, 31, 32, 33, 34, 35, 36, 37, 38, 39, 40, 41, 42, 43, 44, 45, 46, 47, 48, 49 or 50 wt %); i) a mixture of encapsulation aids consisting of corn syrup solids and euphorbia cerifera (candelilla) wax (e.g., 7.0, 7.02, 7.04, 7.06, 7.08, 7.1, 7.12, 7.14, 7.16, 7.18, 7.2, 7.22, 7.24, 7.26, 7.28, 7.3, 7.32, 7.34, 7.36, 7.38, 7.4, 7.42, 7.44, 7.46, 7.48, 7.5, 7.52, 7.54, 7.56, 7.58, 7.6, 7.62, 7.64, 7.66, 7.68, 7.7, 7.72, 7.74, 7.76, 7.78, 7.8, 7.82, 7.84, 7.86, 7.88, 7.9, 7.92, 7.94, 7.96, 7.98, 8.0, 8.02, 8.04, 8.06, 8.08, 8.1, 8.12, 8.14, 8.16, 8.18, 8.2, 8.22, 8.24, 8.26, 8.28, 8.3, 8.32, 8.34, 8.36, 8.38, 8.4, 8.42, 8.44, 8.46, 8.48, 8.5, 8.52, 8.54, 8.56, 8.58, 8.6, 8.62, 8.64, 8.66, 8.68, 8.7, 8.72, 8.74, 8.76, 8.78, 8.8, 8.82, 8.84, 8.86, 8.88, 8.9, 8.92, 8.94, 8.96, 8.98, 9.0, 9.02, 9.04, 9.06, 9.08, 9.1, 9.12, 9.14, 9.16, 9.18, 9.2, 9.22, 9.24, 9.26, 9.28, 9.3, 9.32, 9.34, 9.36, 9.38, 9.4, 9.42, 9.44, 9.46, 9.48, 9.5, 9.52, 9.54, 9.56, 9.58, 9.6, 9.62, 9.64, 9.66, 9.68, 9.7, 9.72, 9.74, 9.76, 9.78, 9.8, 9.82, 9.84, 9.86, 9.88, 9.9, 9.92, 9.94, 9.96, 9.98, or 10.0 total wt %); j) an aqueous phase thickener (e.g., 0.1, 0.12, 0.14, 0.16, 0.18, 0.2, 0.22, 0.24, 0.26, 0.28, 0.3, 0.32, 0.34, 0.36, 0.38, 0.4, 0.42, 0.44, 0.46, 0.48, 0.5, 0.52, 0.54, 0.56, 0.58, 0.6, 0.62, 0.64, 0.66, 0.68, 0.7, 0.72, 0.74, 0.76, 0.78, 0.8, 0.82, 0.84, 0.86, 0.88, 0.9, 0.92, 0.94, 0.96, 0.98, 1.0, 1.02, 1.04, 1.06, 1.08, 1.1, 1.12, 1.14, 1.16, 1.18, 1.2, 1.22, 1.24, 1.26, 1.28, 1.3, 1.32, 1.34, 1.36, 1.38, 1.4, 1.42, 1.44, 1.46, 1.48, 1.5, 1.52, 1.54, 1.56, 1.58, 1.6, 1.62, 1.64, 1.66, 1.68, 1.7, 1.72, 1.74, 1.76, 1.78, 1.8, 1.82, 1.84, 1.86, 1.88, 1.9, 1.92, 1.94, 1.96, 1.98, or 2.0 wt %); k) hesperidin (e.g., 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 wt %); and l) water.

In embodiments is provided a topical composition including: a) a mixture of barrier lipids consisting of cholesterol, one or more ceramides, and one or more free fatty acids (e.g., 1.0, 1.1, 1.2, 1.3, 1.4, 1.5, 1.6, 1.7, 1.8, 1.9, 2.0, 2.02, 2.04, 2.06, 2.08, 2.1, 2.12, 2.14, 2.16, 2.18, 2.2, 2.22, 2.24, 2.26, 2.28, 2.3, 2.32, 2.34, 2.36, 2.38, 2.4, 2.42, 2.44, 2.46, 2.48, 2.5, 2.52, 2.54, 2.56, 2.58, 2.6, 2.62, 2.64, 2.66, 2.68, 2.7, 2.72, 2.74, 2.76, 2.78, 2.8, 2.82, 2.84, 2.86, 2.88, 2.9, 2.92, 2.94, 2.96, 2.98, 3.0, 3.02, 3.04, 3.06, 3.08, 3.1, 3.12, 3.14, 3.16, 3.18, 3.2, 3.22, 3.24, 3.26, 3.28, 3.3, 3.32, 3.34, 3.36, 3.38, 3.4, 3.42, 3.44, 3.46, 3.48, 3.5, 3.52, 3.54, 3.56, 3.58, 3.6, 3.62, 3.64, 3.66, 3.68, 3.7, 3.72, 3.74, 3.76, 3.78, 3.8, 3.82, 3.84, 3.86, 3.88, 3.9, 3.92, 3.94, 3.96, 3.98, 4.0, 4.02, 4.04, 4.06, 4.08, 4.1, 4.12, 4.14, 4.16, 4.18, 4.2, 4.22, 4.24, 4.26, 4.28, 4.3, 4.32, 4.34, 4.36, 4.38, 4.4, 4.42, 4.44, 4.46, 4.48, 4.5, 4.52, 4.54, 4.56, 4.58, 4.6, 4.62, 4.64, 4.66, 4.68, 4.7, 4.72, 4.74, 4.76, 4.78, 4.8, 4.82, 4.84, 4.86, 4.88, 4.9, 4.92, 4.94, 4.96, 4.98, 5.0, 5.02, 5.04, 5.06, 5.08, 5.1, 5.12, 5.14, 5.16, 5.18, 5.2, 5.22, 5.24, 5.26, 5.28, 5.3, 5.32, 5.34, 5.36, 5.38, 5.4, 5.42, 5.44, 5.46, 5.48, 5.5, 5.52, 5.54, 5.56, 5.58, 5.6, 5.62, 5.64, 5.66, 5.68, 5.7, 5.72, 5.74, 5.76, 5.78, 5.8, 5.82, 5.84, 5.86, 5.88, 5.9, 5.92, 5.94, 5.96, 5.98, 6.0, 6.02, 6.04, 6.06, 6.08, 6.1, 6.12, 6.14, 6.16, 6.18, 6.2, 6.22, 6.24, 6.26, 6.28, 6.3, 6.32, 6.34, 6.36, 6.38, 6.4, 6.42, 6.44, 6.46, 6.48, 6.5, 6.52, 6.54, 6.56, 6.58, 6.6, 6.62, 6.64, 6.66, 6.68, 6.7, 6.72, 6.74, 6.76, 6.78, 6.8, 6.82, 6.84, 6.86, 6.88, 6.9, 6.92, 6.94, 6.96, 6.98, 7.0, 7.02, 7.04, 7.06, 7.08, 7.1, 7.12, 7.14, 7.16, 7.18, 7.2, 7.22, 7.24, 7.26, 7.28, 7.3, 7.32, 7.34, 7.36, 7.38, 7.4, 7.42, 7.44, 7.46, 7.48, 7.5, 7.52, 7.54, 7.56, 7.58, 7.6, 7.62, 7.64, 7.66, 7.68, 7.7, 7.72, 7.74, 7.76, 7.78, 7.8, 7.82, 7.84, 7.86, 7.88, 7.9, 7.92, 7.94, 7.96, 7.98, 8.0, 8.02, 8.04, 8.06, 8.08, 8.1, 8.12, 8.14, 8.16, 8.18, 8.2, 8.22, 8.24, 8.26, 8.28, 8.3, 8.32, 8.34, 8.36, 8.38, 8.4, 8.42, 8.44, 8.46, 8.48, 8.5, 8.52, 8.54, 8.56, 8.58, 8.6, 8.62, 8.64, 8.66, 8.68, 8.7, 8.72, 8.74, 8.76, 8.78, 8.8, 8.82, 8.84, 8.86, 8.88, 8.9, 8.92, 8.94, 8.96, 8.98, 9.0, 9.02, 9.04, 9.06, 9.08, 9.1, 9.12, 9.14, 9.16, 9.18, 9.2, 9.22, 9.24, 9.26, 9.28, 9.3, 9.32, 9.34, 9.36, 9.38, 9.4, 9.42, 9.44, 9.46, 9.48, 9.5, 9.52, 9.54, 9.56, 9.58, 9.6, 9.62, 9.64, 9.66, 9.68, 9.7, 9.72, 9.74, 9.76, 9.78, 9.8, 9.82, 9.84, 9.86, 9.88, 9.9, 9.92, 9.94, 9.96, 9.98, or 10 total wt %); b) a skin conditioner (e.g., 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2.0, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3, 2.31, 2.32, 2.33, 2.34, 2.35, 2.36, 2.37, 2.38, 2.39, 2.4, 2.41, 2.42, 2.43, 2.44, 2.45, 2.46, 2.47, 2.48, 2.49, 2.5, 2.51, 2.52, 2.53, 2.54, 2.55, 2.56, 2.57, 2.58, 2.59, 2.6, 2.61, 2.62, 2.63, 2.64, 2.65, 2.66, 2.67, 2.68, 2.69, 2.7, 2.71, 2.72, 2.73, 2.74, 2.75, 2.76, 2.77, 2.78, 2.79, 2.8, 2.81, 2.82, 2.83, 2.84, 2.85, 2.86, 2.87, 2.88, 2.89, 2.9, 2.91, 2.92, 2.93, 2.94, 2.95, 2.96, 2.97, 2.98, 2.99, or 3.0 wt %); c) a chelating agent (e.g., 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1.0 wt %); d) humectant (e.g., 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0, 25.0, 26.0, 27.0, 28.0, 29.0 or 30.0 wt %); e) glyceryl stearate PEG-100 emulsifier (e.g., 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2.0, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3, 2.31, 2.32, 2.33, 2.34, 2.35, 2.36, 2.37, 2.38, 2.39, 2.4, 2.41, 2.42, 2.43, 2.44, 2.45, 2.46, 2.47, 2.48, 2.49, 2.5, 2.51, 2.52, 2.53, 2.54, 2.55, 2.56, 2.57, 2.58, 2.59, 2.6, 2.61, 2.62, 2.63, 2.64, 2.65, 2.66, 2.67, 2.68, 2.69, 2.7, 2.71, 2.72, 2.73, 2.74, 2.75, 2.76, 2.77, 2.78, 2.79, 2.8, 2.81, 2.82, 2.83, 2.84, 2.85, 2.86, 2.87, 2.88, 2.89, 2.9, 2.91, 2.92, 2.93, 2.94, 2.95, 2.96, 2.97, 2.98, 2.99, or 3.0 wt %); f) a mixture of emollients consisting of petrolatum and squalane (e.g., 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24.0, 24.5, 25.0, 25.5, 26.0, 26.5, 27.0, 27.5, 28.0, 28.5, 29.0, 29.5, or 30.0 wt %); g) a mixture of preservatives consisting of phenoxyethanol and sorbic acid (e.g., 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, or 1.5 total wt %); h) citric acid (e.g., 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.55, 0.6, 0.65, 0.7, 0.75, 0.8, 0.9, 1, 2, 3, 4, 5, 6, 7, 8, 9, 10, 11, 12, 13, 14, 15, 16, 17, 18, 19, 20, 21, 2, 23, 24, 25, 26, 27, 28, 29, 30, 31, 32, 33, 34, 35, 36, 37, 38, 39, 40, 41, 42, 43, 44, 45, 46, 47, 48, 49 or 50 wt %); i) a mixture of encapsulation aids consisting of corn syrup solids and euphorbia cerifera (candelilla) wax (e.g., 7.0, 7.02, 7.04, 7.06, 7.08, 7.1, 7.12, 7.14, 7.16, 7.18, 7.2, 7.22, 7.24, 7.26, 7.28, 7.3, 7.32, 7.34, 7.36, 7.38, 7.4, 7.42, 7.44, 7.46, 7.48, 7.5, 7.52, 7.54, 7.56, 7.58, 7.6, 7.62, 7.64, 7.66, 7.68, 7.7, 7.72, 7.74, 7.76, 7.78, 7.8, 7.82, 7.84, 7.86, 7.88, 7.9, 7.92, 7.94, 7.96, 7.98, 8.0, 8.02, 8.04, 8.06, 8.08, 8.1, 8.12, 8.14, 8.16, 8.18, 8.2, 8.22, 8.24, 8.26, 8.28, 8.3, 8.32, 8.34, 8.36, 8.38, 8.4, 8.42, 8.44, 8.46, 8.48, 8.5, 8.52, 8.54, 8.56, 8.58, 8.6, 8.62, 8.64, 8.66, 8.68, 8.7, 8.72, 8.74, 8.76, 8.78, 8.8, 8.82, 8.84, 8.86, 8.88, 8.9, 8.92, 8.94, 8.96, 8.98, 9.0, 9.02, 9.04, 9.06, 9.08, 9.1, 9.12, 9.14, 9.16, 9.18, 9.2, 9.22, 9.24, 9.26, 9.28, 9.3, 9.32, 9.34, 9.36, 9.38, 9.4, 9.42, 9.44, 9.46, 9.48, 9.5, 9.52, 9.54, 9.56, 9.58, 9.6, 9.62, 9.64, 9.66, 9.68, 9.7, 9.72, 9.74, 9.76, 9.78, 9.8, 9.82, 9.84, 9.86, 9.88, 9.9, 9.92, 9.94, 9.96, 9.98, or 10.0 total wt %); j) an aqueous phase thickener (e.g., 0.1, 0.12, 0.14, 0.16, 0.18, 0.2, 0.22, 0.24, 0.26, 0.28, 0.3, 0.32, 0.34, 0.36, 0.38, 0.4, 0.42, 0.44, 0.46, 0.48, 0.5, 0.52, 0.54, 0.56, 0.58, 0.6, 0.62, 0.64, 0.66, 0.68, 0.7, 0.72, 0.74, 0.76, 0.78, 0.8, 0.82, 0.84, 0.86, 0.88, 0.9, 0.92, 0.94, 0.96, 0.98, 1.0, 1.02, 1.04, 1.06, 1.08, 1.1, 1.12, 1.14, 1.16, 1.18, 1.2, 1.22, 1.24, 1.26, 1.28, 1.3, 1.32, 1.34, 1.36, 1.38, 1.4, 1.42, 1.44, 1.46, 1.48, 1.5, 1.52, 1.54, 1.56, 1.58, 1.6, 1.62, 1.64, 1.66, 1.68, 1.7, 1.72, 1.74, 1.76, 1.78, 1.8, 1.82, 1.84, 1.86, 1.88, 1.9, 1.92, 1.94, 1.96, 1.98, or 2.0 wt %); k) hesperidin (e.g., 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 wt %); and l) water.

In embodiments is provided a composition including: a) a mixture of barrier lipids selected from the group consisting of cholesterol, ceramides, free fatty acids and mixtures of two or more thereof (e.g., 1.0, 1.1, 1.2, 1.3, 1.4, 1.5, 1.6, 1.7, 1.8, 1.9, 2.0, 2.02, 2.04, 2.06, 2.08, 2.1, 2.12, 2.14, 2.16, 2.18, 2.2, 2.22, 2.24, 2.26, 2.28, 2.3, 2.32, 2.34, 2.36, 2.38, 2.4, 2.42, 2.44, 2.46, 2.48, 2.5, 2.52, 2.54, 2.56, 2.58, 2.6, 2.62, 2.64, 2.66, 2.68, 2.7, 2.72, 2.74, 2.76, 2.78, 2.8, 2.82, 2.84, 2.86, 2.88, 2.9, 2.92, 2.94, 2.96, 2.98, 3.0, 3.02, 3.04, 3.06, 3.08, 3.1, 3.12, 3.14, 3.16, 3.18, 3.2, 3.22, 3.24, 3.26, 3.28, 3.3, 3.32, 3.34, 3.36, 3.38, 3.4, 3.42, 3.44, 3.46, 3.48, 3.5, 3.52, 3.54, 3.56, 3.58, 3.6, 3.62, 3.64, 3.66, 3.68, 3.7, 3.72, 3.74, 3.76, 3.78, 3.8, 3.82, 3.84, 3.86, 3.88, 3.9, 3.92, 3.94, 3.96, 3.98, 4.0, 4.02, 4.04, 4.06, 4.08, 4.1, 4.12, 4.14, 4.16, 4.18, 4.2, 4.22, 4.24, 4.26, 4.28, 4.3, 4.32, 4.34, 4.36, 4.38, 4.4, 4.42, 4.44, 4.46, 4.48, 4.5, 4.52, 4.54, 4.56, 4.58, 4.6, 4.62, 4.64, 4.66, 4.68, 4.7, 4.72, 4.74, 4.76, 4.78, 4.8, 4.82, 4.84, 4.86, 4.88, 4.9, 4.92, 4.94, 4.96, 4.98, 5.0, 5.02, 5.04, 5.06, 5.08, 5.1, 5.12, 5.14, 5.16, 5.18, 5.2, 5.22, 5.24, 5.26, 5.28, 5.3, 5.32, 5.34, 5.36, 5.38, 5.4, 5.42, 5.44, 5.46, 5.48, 5.5, 5.52, 5.54, 5.56, 5.58, 5.6, 5.62, 5.64, 5.66, 5.68, 5.7, 5.72, 5.74, 5.76, 5.78, 5.8, 5.82, 5.84, 5.86, 5.88, 5.9, 5.92, 5.94, 5.96, 5.98, 6.0, 6.02, 6.04, 6.06, 6.08, 6.1, 6.12, 6.14, 6.16, 6.18, 6.2, 6.22, 6.24, 6.26, 6.28, 6.3, 6.32, 6.34, 6.36, 6.38, 6.4, 6.42, 6.44, 6.46, 6.48, 6.5, 6.52, 6.54, 6.56, 6.58, 6.6, 6.62, 6.64, 6.66, 6.68, 6.7, 6.72, 6.74, 6.76, 6.78, 6.8, 6.82, 6.84, 6.86, 6.88, 6.9, 6.92, 6.94, 6.96, 6.98, 7.0, 7.02, 7.04, 7.06, 7.08, 7.1, 7.12, 7.14, 7.16, 7.18, 7.2, 7.22, 7.24, 7.26, 7.28, 7.3, 7.32, 7.34, 7.36, 7.38, 7.4, 7.42, 7.44, 7.46, 7.48, 7.5, 7.52, 7.54, 7.56, 7.58, 7.6, 7.62, 7.64, 7.66, 7.68, 7.7, 7.72, 7.74, 7.76, 7.78, 7.8, 7.82, 7.84, 7.86, 7.88, 7.9, 7.92, 7.94, 7.96, 7.98, 8.0, 8.02, 8.04, 8.06, 8.08, 8.1, 8.12, 8.14, 8.16, 8.18, 8.2, 8.22, 8.24, 8.26, 8.28, 8.3, 8.32, 8.34, 8.36, 8.38, 8.4, 8.42, 8.44, 8.46, 8.48, 8.5, 8.52, 8.54, 8.56, 8.58, 8.6, 8.62, 8.64, 8.66, 8.68, 8.7, 8.72, 8.74, 8.76, 8.78, 8.8, 8.82, 8.84, 8.86, 8.88, 8.9, 8.92, 8.94, 8.96, 8.98, 9.0, 9.02, 9.04, 9.06, 9.08, 9.1, 9.12, 9.14, 9.16, 9.18, 9.2, 9.22, 9.24, 9.26, 9.28, 9.3, 9.32, 9.34, 9.36, 9.38, 9.4, 9.42, 9.44, 9.46, 9.48, 9.5, 9.52, 9.54, 9.56, 9.58, 9.6, 9.62, 9.64, 9.66, 9.68, 9.7, 9.72, 9.74, 9.76, 9.78, 9.8, 9.82, 9.84, 9.86, 9.88, 9.9, 9.92, 9.94, 9.96, 9.98, or 10 total wt %) b) hesperidin (e.g., 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 wt %); c) an acid buffer (e.g., 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.55, 0.6, 0.65, 0.7, 0.75, 0.8, 0.9, 1, 2, 3, 4, 5, 6, 7, 8, 9, 10, 11, 12, 13, 14, 15, 16, 17, 18, 19, 20, 21, 2, 23, 24, 25, 26, 27, 28, 29, 30, 31, 32, 33, 34, 35, 36, 37, 38, 39, 40, 41, 42, 43, 44, 45, 46, 47, 48, 49 or 50 wt %); and d) at least one glucocorticoid (e.g., 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2.0, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3, 2.31, 2.32, 2.33, 2.34, 2.35, 2.36, 2.37, 2.38, 2.39, 2.4, 2.41, 2.42, 2.43, 2.44, 2.45, 2.46, 2.47, 2.48, 2.49, 2.5, 2.51, 2.52, 2.53, 2.54, 2.55, 2.56, 2.57, 2.58, 2.59, 2.6, 2.61, 2.62, 2.63, 2.64, 2.65, 2.66, 2.67, 2.68, 2.69, 2.7, 2.71, 2.72, 2.73, 2.74, 2.75, 2.76, 2.77, 2.78, 2.79, 2.8, 2.81, 2.82, 2.83, 2.84, 2.85, 2.86, 2.87, 2.88, 2.89, 2.9, 2.91, 2.92, 2.93, 2.94, 2.95, 2.96, 2.97, 2.98, 2.99, or 3.0 wt %).

Any of the methods including administration or use of hesperidin described herein, may instead administer or use an analog of hesperidin. Any of the methods including administration or use of hesperidin described herein, may instead administer or use a pharmaceutically acceptable salt of hesperidin. Any of the methods including administration or use of hesperidin described herein, may instead administer or use a prodrug of hesperidin.

### III. METHODS OF USE

The topical composition of the present invention is generally prepared by conventional methods such as are known in the art of making topical compositions. Such methods typically involve mixing of the ingredients in one or more steps to a relatively uniform state, with or without heating, cooling, application of vacuum, and the like.

The topical composition is useful for regulating or improving skin condition, including regulating visible or tactile wrinkles or discontinuities in skin and those associated with skin aging, e.g., visible and/or tactile scaling, wrinkles or discontinuities in skin texture or color. Such wrinkles, scale or discontinuities may be induced or caused by internal factors (e.g., chronological aging and other biochemical changes from within the skin) or external factors (e.g., photo-aging) (e.g., ultraviolet radiation, environmental pollution, wind, heat, low humidity, harsh surfactants, and abrasives).

Regulating skin conditions by improving barrier function, antimicrobial defense, or SC integrity can be carried out prophylactically or therapeutically. Prophylactic regulation includes delaying, minimizing, or preventing the development of visible or tactile wrinkles or discontinuities in skin. Therapeutic regulation, on the other hand, includes ameliorating, diminishing, minimizing or effacing such wrinkles, scale, or discontinuities. Regulating skin conditions (e.g. treating abnormal barrier function, treating aged skin, treating dry skin) involves improving skin appearance and feel, by providing a smoother, more even appearance, or feel and reducing signs of aging.

To use a topical composition of this invention, one can topically apply to the skin a safe and effective amount of the composition. The applied amount, the frequency of application and the period of use vary widely depending upon the active levels of a given composition and the level of regulation desired, e.g., in light of the level of skin ageing present in the subject and the rate of further skin ageing.

A wide range of quantities of the compositions of the present invention can be employed to provide a skin appearance and/or feel benefit. Quantities of the compositions typically applied per application are from about 0.1 mg/cm² to about 10 mg/cm², e.g., 2 mg/cm². Typically, a composition can be used once or twice per day. However, application rates can vary from about once per week up to about three times per day or more.

Regulating skin conditions is preferably performed by applying a composition in the form of a skin lotion, cream, cosmetic, or the like which is intended to be left on the skin for an extended period for some aesthetic, prophylactic, therapeutic or other benefit (i.e., a “leave-on” composition). As used herein, “leave-on” compositions exclude rinse-off skin cleansing products. After applying the composition to the skin, the leave-on composition is preferably left on the skin for a period of at least about 15 minutes, 30 minutes, 1 hour, or up to about 12 hours.

With regard to the epidermal barrier function, the anti-inflammatory composition described above is largely preventive, since ‘fixing’ the barrier is anti-inflammatory in either aging or young skin. This composition includes not only the barrier lipids in a barrier-enhancing molar ratio and pH, but also includes topical glucocorticoids or immunomodulators, and together such a combination reduces inflammation and prevents disease flares. It also prevents the emergence of epidermal side effects, such as rebound flares and tachyphylaxis, due to the co-administered glucocorticoids or immunomodulators. With regard to inflammatory diseases that are attributable, at least in part, to epidermal intrinsic/chronologic aging, these compositions are useful for the treatment of conditions including a) xerosis; b) xerotic eczema; c) winter itch; d) nummular eczema; e) seborrheic dermatitis; f) occupational dermatitis; g) hand eczema; h) stasis dermatitis; and any other eczematous condition occurring in the aging population.

Another aspect of the invention is directed to a method of treating inflammation by administering an effective amount of the above anti-inflammatory composition as described herein to at least a portion of affected skin in need thereof.

A further aspect of the invention is directed to a method of preventing or treating aged skin, including administering one of the topical compositions described above to at least a portion of aged skin, in an amount effective to relieve or reverse a clinical indication of skin aging. In embodiments, the method is directed to a method of treating aged skin, including administering one of the topical compositions described above to at least a portion of aged skin, in an amount effective to relieve or reverse a clinical indication of skin aging. A clinical indication of skin aging can be selected from the group consisting of eczema, xerosis, xerotic eczema, winter itch, nummular eczema, seborrheic dermatitis, occupational dermatitis, hand eczema, and stasis dermatitis. In embodiments, the method includes treating functional abnormalities in chronologically- and/or photo-aged skin, wherein the functional abnormalities may include reduced stratum corneum hydration, reduced stratum corneum cohesion, increased abnormal desquamation, increased skin scaling, increased occurrence of skin infections, increased susceptibility to skin infections, increased susceptibility of inflammatory dermatoses, and/or increased occurrence of inflammatory dermatoses. In embodiments, the method includes improving (i.e. increasing) stratum corneum hydration, improving (i.e. increasing) stratum corneum cohesion, inhibiting (i.e. reducing) abnormal desquamation, inhibiting (i.e. reducing) excessive scaling), reducing occurrence of skin infections, reducing susceptibility to skin infections, reducing susceptibility of inflammatory dermatoses, and/or reducing occurrence of inflammatory dermatoses. In embodiments, the method includes treating functional abnormalities in chronologically-aged skin. In embodiments, the method includes treating functional abnormalities in photo-aged skin.

In embodiments, the method includes administering to a patient one of the topical compositions described herein begins when the patient is about 40 years old. In embodiments, the patient is 36 years old. In embodiments, the patient is 37 years old. In embodiments, the patient is 38 years old. In embodiments, the patient is 39 years old. In embodiments, the patient is 40 years old. In embodiments, the patient is 41 years old. In embodiments, the patient is 42 years old. In embodiments, the patient is 43 years old. In embodiments, the patient is 44 years old.

Another aspect of the invention is directed to a method of preventing or reversing dermal side effects of systemic or topical glucocorticoid treatment, including treating at least a portion of affected skin with one of the compositions described above, in an amount effective to prevent or reverse a dermal clinical indication of glucocorticoid treatment. A dermal clinical indication of glucocorticoid treatment can be selected from the group consisting of inflammation, tachyphylaxis, disease flares, and rebound flares.

In an aspect is provided a method of preventing or reversing a dermal side effect of topical or systemic immunomodulator treatment, including treating at least a portion of affected skin with one of the compositions described above, in an amount effective to prevent or reverse dermal clinical indications of immunomodulators treatment. In embodiments, the immunomodulator include pimecrolimus and/or tacrolimus. In embodiments, the immunomodulator is a topical calcineurin inhibitor (e.g., pimecrolimus and/or tacrolimus). Examples of additional topical calcineurin inhibitors can be found in Breuer et al. (Am. J. Clin. Dermatol. 2005; 6(2):65-77) and is incorporated herein in its entirety. Dermal clinical indications of glucocorticoid treatment can be selected from the group consisting of inflammation, tachyphylaxis, disease flares, and rebound flares.

Yet another aspect of the invention is directed to a method for the treatment of dry skin or abnormal epidermal barrier function associated with oral hypolipodemic agent treatment, for example, statin treatment, including administering one of the topical compositions described above to at least a portion of affected skin of a subject, in an amount effective to relieve or reverse clinical indications of hypolipodemic treatment. In embodiments, the subject has been administered a hypolipodemic agent, such as a statin, or is expected to receive such treatment. In embodiments, the subject has been co-administered a hypolipodemic agent. Hypolipodemic agents other than statins include fibrates, niacin, bile acid sequestrants, ezetimibe, lomitapide, phytosterols, orlistat, cholesteryl ester transfer protein inhibitors, and squalane synthase inhibitors.

In embodiments, the composition is for use in treating dry skin in a subject in need thereof. In embodiments, the composition is for use in treating abnormal epidermal barrier function in a subject in need thereof. In embodiments, the composition is for use in treating an inflammatory condition in a subject in need thereof. In embodiments, the composition is for use in treating a clinical indication of skin aging in a subject in need thereof. In embodiments, the clinical indication of skin aging is eczema, xerosis, xerotic eczema, winter itch, nummular eczema, seborrheic dermatitis, occupational dermatitis, hand eczema, or stasis dermatitis. In embodiments, the composition is for use in treating a dermal clinical indication of glucocorticoid treatment in a subject in need thereof. In embodiments, the dermal clinical indication of glucocorticoid treatment is inflammation, tachyphylaxis, disease flares, or rebound flares. In embodiments, the composition is for use in treating a clinical indication of hypolipodemic treatment in a subject in need thereof.

One aspect of the invention is directed to a topical composition comprising citrus bioflavonoid (e.g., hesperidin, or an analog, pharmaceutically acceptable salt, or prodrug thereof) in the absence of lipids providing desirable restorative effects on the epidermal permeability barrier in aged skin, as provided herein. In embodiments, the citrus bioflavonoid is hesperidin.

A further aspect of the invention is directed to a method of treating aged skin, comprising administering one of the topical compositions comprising citrus bioflavonoid (e.g., hesperidin, or an analog, pharmaceutically acceptable salt, or prodrug thereof) in the absence of lipids to at least a portion of aged skin, in an amount effective to relieve or reverse a clinical indication of skin aging. In embodiments, the citrus bioflavonoid is hesperidin.

Another aspect of the invention is directed to a method of preventing or reversing an epidermal side effect of systemic or topical glucocorticoid treatment, comprising treating at least a portion of affected skin with one of the topical compositions comprising citrus bioflavonoid (e.g., hesperidin, or an analog, pharmaceutically acceptable salt, or prodrug thereof) in the absence of lipids, in an amount effective to prevent or reverse epidermal a clinical indication of glucocorticoid side effects. In embodiments, the citrus bioflavonoid is hesperidin.

One aspect of the invention is directed to a topical composition comprising hesperidin in the absence of lipids providing desirable restorative effects on the epidermal permeability barrier in aged skin, as provided herein.

A further aspect of the invention is directed to a method of treating aged skin, comprising administering one of the topical compositions comprising hesperidin in the absence of lipids to at least a portion of aged skin, in an amount effective to relieve or reverse clinical indications of skin aging.

Another aspect of the invention is directed to a method of preventing or reversing epidermal side effects of systemic or topical glucocorticoid treatment, comprising treating at least a portion of affected skin with one of the topical compositions comprising hesperidin in the absence of lipids, in an amount effective to prevent or reverse epidermal clinical indications of glucocorticoid side effects.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

Citrus bioflavonoids, such as orange peel extract, reportedly exhibit beneficial effects for skin pigmentation, inflammation, and ultraviolet (UV) protection, as well as stimulating keratinocyte proliferation. We have recently demonstrated that topical hesperidin improves epidermal permeability barrier function in young normal murine skin, and have explored the responsible mechanisms. Topical hesperidin significantly accelerated young murine skin barrier recovery after acute barrier abrogation; which correlated with stimulation of both epidermal proliferation and differentiation, as well as enhanced lamellar body (lipid) secretion. These results indicated that topical hesperidin enhances epidermal permeability barrier homeostasis in normal skin by stimulating epidermal proliferation and differentiation, and by stimulating lamellar body secretion. However, the benefits of topical citrus bioflavonoids such as hesperidin for aged skin have not heretofore been disclosed.

As noted in the Background section, synthesis of the three key barrier-related lipids, cholesterol, ceramides (including pseudo-ceramides), and free fatty acids, requires their respective rate-limiting enzymes, HMGCoA, SPT1, and FAS. Basal mRNA levels for all three key lipid synthetic enzymes, and particularly HMGCoA reductase, were observed to be lower in aged epidermis as compared with young epidermis, consistent with concept that the lower lipid synthesis rates in aged epidermis reflect the reduced expression of their synthetic enzymes. Surprisingly, we have now demonstrated that topical hesperidin treatment significantly increased the mRNA levels of HMGCoA, SPT1 and FAS in aged mouse epidermis, as assessed by Q-PCR.

Thus, topical applications of hesperidin improve multiple key epidermal functions in aged mouse skin. After 9 days of treatment, the gross appearance of mouse skin treated with vehicle and hesperidin appeared similar. Histological analysis showed that aged epidermis was thinner than young epidermis; whereas proliferating cell nuclear antigen (PCNA) staining indicated that aged epidermis displayed less robust proliferative activity as compared with young epidermis; hesperidin treatment did not stimulate epidermal proliferation in aged skin, as indicated by PCNA-positive cells per cm epidermal length (2.70±0.10 vs. 2.45±0.13 for vehicle-treated vs. hesperidin-treated skin, NS; 3.46±0.17 for young skin; young vs. vehicle- or hesperidin-treated aged skin, P<0.001). These results indicate that topical hesperidin does not stimulate epidermal proliferation in aged mice.

After 9 days of topical hesperidin treatment, baseline SC hydration in hesperidin-treated mice also was no different from that in vehicle-treated mice (60.77±1.32 for vehicle-treated vs. 58.80±2.27 for hesperidin-treated). However, skin surface pH significantly declined in hesperidin-treated skin compared with vehicle-treated skin. Although basal transepidermal water loss rates increased slightly in hesperidin-treated skin as compared with vehicle-treated skin, these levels still fell well within the normal range of young skin. Consistent with previous findings in young mice, topical hesperidin significantly accelerated barrier recovery at both 2 and 4 hours after acute barrier disruption of aged skin. These results demonstrate that topical hesperidin improves epidermal permeability barrier homeostasis, while also lowering skin surface pH in aged murine skin.

We next examined the basis for improved barrier function and acidification in aged epidermis. Our previous studies demonstrated that topical hesperidin stimulates epidermal differentiation, accounting in part for improved epidermal permeability barrier homeostasis in young mice. Hence, we next assessed whether topical hesperidin also stimulates epidermal differentiation in aged epidermis. Topical hesperidin significantly increased the mRNA levels of filaggrin and loricrin in aged mouse epidermis, consistent with the results of immunostaining. Consistently, hesperidin also increased the mRNA levels of filaggrin, involucrin, and loricrin in adult keratinocyte cultures. These results indicate that hesperidin stimulates epidermal differentiation, providing one potential mechanism whereby hesperidin improves barrier function in aged skin.

Epidermal lipid synthesis is required for the formation and maintenance of the epidermal permeability barrier. Synthesis of three key barrier-related lipids, cholesterol, ceramides, and fatty acids requires their respective rate-limiting enzymes 3-hydroxy-3-methyl-glutaryl-CoA reductase (HMGCoA), serine palmitoyltransferase 1 (SPT1), and fatty acid synthase (FAS). Basal mRNA levels for all three key lipid synthetic enzymes were lower in aged as compared with young epidermis, consistent with the concept that the lower lipid synthesis rates in aged epidermis could reflect the reduced expression of their synthetic enzymes. Topical hesperidin treatment significantly increased the mRNA levels of HMGCoA, SPT1, and FAS in aged mouse epidermis, as assessed by quantitative reverse transcriptase in real time (Q-PCR).

Epidermal permeability barrier function depends on newly synthesized epidermal lipids delivered to the SC through the secretion of lamellar bodies from the stratum granulosum. Therefore, we next assessed whether topical hesperidin stimulates lamellar body formation and/or secretion. As ATP-binding cassette transporter 12 (ABCA12), a trans-membrane glycosylceramide transporter, is required for normal lamellar body assembly, we next evaluated the changes in epidermal mRNA levels of ABCA12 in hesperidin-treated aged epidermis. Although untreated aged epidermis displayed lower levels of ABCA12 mRNA in comparison with young mice, topical hesperidin induced a marked increase in ABCA12 mRNA expression in aged mouse epidermis and adult keratinocyte cultures. Although the density of lamellar bodies did not increase in aged epidermis after hesperidin treatment, quantitative analyses revealed that the extent of lamellar body secretion was enhanced by topical hesperidin treatment. In comparison with young epidermis, the increased number of lamellar bodies in aged epidermis is likely because of the retardation of secretion. Together, these results suggest that hesperidin induced an increase in ABCA12 mRNA expression that results in an apparent acceleration in the delivery of newly synthesized lipids to the SC.

Both epidermal sodium/hydrogen exchanger 1 (NHE1) and secretory phospholipase A2 (sPLA2; in particular SPLAg2f) are key factors that selectively influence the pH of the SC. Previous studies from our group have shown that aged skin exhibits higher pH, at least partly owing to reduced NHE1 expression. To determine whether the hesperidin-induced acidification of the pH of the SC results from the upregulation of NHE1 and/or the parallel acidifying mechanism, sPLA2g2f, we next assessed the changes in epidermal mRNA levels of these two genes in aged epidermis after herperidin treatments by Q-PCR. Topical hesperidin provoked a marked elevation in mRNA levels for both NHE1 and sPLA2g2f in aged epidermis. These results suggest that hesperidin-induced acidification of aged epidermis results from stimulation of NHE1 and sPLA2g2f, accounting for the lower skin surface pH, and likely improved epidermal permeability barrier homeostasis in hesperidin-treated aged mouse skin.

Epidermal permeability barrier and antimicrobial function are co-regulated and independent. Aged humans are predisposed to develop both cutaneous and extracutaneous infections, and expression of the epidermal cathelicidin antimicrobial peptide CAMP/LL37 is reduced in aged skin. To determine whether hesperidin enhances epidermal antimicrobial defense, we next assessed changes in the mRNA levels of mouse beta-defensin 3 (mBD3), a homolog of human beta-defensin 2 (hBD2), following hesperidin treatment. Hesperidin treatment significantly increased epidermal mBD3 mRNA levels. To further validate these in vivo results, the effects of hesperidin on antimicrobial mRNA expression were evaluated in cultured keratinocytes from aged human skin. Although no changes in constitutive hBD2 mRNA expression were observed, the addition of hesperidin to aged human keratinocyte cultures markedly upregulated not only hBD3 mRNA but also CAMP/LL37 expression. These results demonstrate that hesperidin stimulates antimicrobial peptide mRNA expression in aged keratinocytes.

Thus, we have demonstrated that topical hesperidin improves a wide spectrum of functional abnormalities in aged epidermis, including abnormalities in epidermal permeability barrier function, epidermal differentiation, lipid production, and SC acidification. The antioxidant property of hesperidin is also beneficial. Aged skin displays lower antioxidant capacity and excessive accumulation of oxidative products, and hesperidin shows high antioxidant capacity. We have demonstrated that topical hesperidin applications increased epidermal mRNA levels of antioxidant enzymes such as glutathione reductase and superoxide dismutase in murine skin. Pertinent to antioxidant activity, nuclear factor (erythroid-derived 2)-like 2 (Nrf2), a transcription factor, regulates epidermal differentiation and antioxidant defense. Nrf2 function is impaired in aged heart, and expression levels were lower in aged epidermis. Hesperidin upregulates Nrf2 in the heart and in the aged epidermis. Hence, hesperidin-induced improvement of epidermal permeability barrier function in aged skin might be mediated via Nrf2.

Glucocorticoid Side Effects.

Systemic and topical glucocorticoids (GC) can cause significant adverse effects not only on the dermis, but also on epidermal structure and function. In epidermis, one striking GC-induced alteration in permeability barrier function occurs that can be attributed to an inhibition of epidermal mitogenesis, differentiation and lipid production, features that mimic aged skin. Hesperidin co-applications unexpectedly also prevented the anticipated GC-induced impairments of epidermal permeability barrier homeostasis and stratum corneum (SC) integrity and SC acidification. These preventive effects could be attributed to a significant increase in filaggrin expression, enhanced epidermal β-glucocerebrosidase activity and accelerated lamellar bilayer maturation, the last two likely attributable to a hesperidin-induced reduction in stratum corneum pH. Furthermore, co-applications of hesperidin with GC surprisingly prevented the expected GC-induced inhibition of epidermal proliferation. Finally, topical hesperidin also unexpectedly increased epidermal anti-oxidant enzymes, including glutathione reductase and superoxide dismutase, mRNA expression, which could counteract multiple negative effects of GC on epidermal function. Together, these results show that, surprisingly, topical hesperidin prevents GC-induced epidermal side effects by divergent mechanisms.

A notable unexpected finding that emerged was that topical hesperidin prevented the topical GC-induced changes in skin surface pH. Not only does an acidic pH accelerate epidermal permeability barrier recovery in adult and neonatal barrier maturation, but it also prevents the development of atopic dermatitis in a murine model. To date, hesperidin is the only known agent shown to prevent the GC-induced abnormality in skin surface pH. Although the underlying mechanism(s) remains unknown, the resultant improvement in SC pH could significantly impact epidermal function by acidification-induced increase in 0-glucocerebrosidase and sphingomyelinase activity, which also correlated with accelerated maturation of SC extracellular lamellar bilayers. Thus, a reduced SC pH likely represents another mechanism by which hesperidin prevents the emergence of GC-induced permeability barrier abnormalities.

pH and Stratum Corneum Acidification and Sources of the Acid Mantle.

The acid mantle refers to the highly acidic film of the SC that acts as a deterrent to the entry of bacteria, viruses and other potential contaminants that might penetrate the skin. Although the acid mantle initially was assumed to result from exogenous acidic sources, such as sebum-derived free fatty acids, instead it has now been unexpectedly observed that several endogenous acidifying mechanisms account for up to 2 pH units (a 200-fold increase in protons) of the overall pH of the stratum corneum (SC) (Table 1).

TABLE 1
Impact of Endogenous Acidifying Mechanisms on SC Acidification &
Epidermal Functions. Abbreviations: Chol, cholesterol; CSO4, cholesterol sulfate;
FFA, free fatty acid; FLG, filaggrin; NHE1, Na+/H+ antiporter 1; PCA,
polycarboxylic acid; PL, phospholipids; SO4, sulfate; t-UCA, trans-urocanic acid.

| | | | Known | Changes | |
|---|---|---|---|---|---|
| | | | Impact on | during | Changes |
| | Change in | | Epidermal | Post-Natal | in |
| Acidifying Mechanisms | Bulk pH | Localization | Functions | Development | Disease |
| PL → FFA | ca 1 unit | ? | ↑Barrier, | ↓Neonatal | ? |
| (sPLA2G2F) | | | ↑Cohesion | | |
| NHE1 | ca ¼ | SG-SC | ↑Barrier | ↓Aged | ? |
| | unit | interface | | | |
| FLG → PCA (t-UCA) | ca ½ | ? | ↑Barrier | ? | ? |
| | unit | | | | |
| Melanin | transient | SG-SC | ↑Barrier, | ? | ? |
| persistence/extrusion | | interface | ↑Cohesion | | |
| CSO₄ → Chol (+SO₄⁼) | ? | ? | ↑Cohesion | ? | ? |

The sodium-protein antiporter, type 1 (NHE1), which selectively acidifies extracellular domains at the stratum granulosum (SG)/SC interface, accounts for ca. ¼ unit of the bulk pH of the SC. This critically-important site is where sphingomyelin and glucosylceramides are processed by the acidic pH-dependent enzymes, acidic sphingomyelinase (aSMase) and β-glucocerebrosidase (β-GlcCer'ase) into ceramides, thereby generating the permeability barrier.

Further, secretory phospholipase (sPLA2) releases free fatty acids (FFA) from the sn.2 position of glycerophospholipids (PL). While one subset of sPLA2 releases arachidonic acid that is subsequently converted to eicosanoids, other sPLA2 release FFAs that are required for permeability barrier homeostasis and SC acidification (≈1 pH unit), but only the PLA2G2F isoform is regulated and required for permeability barrier homeostasis. Notably, Pla2g2f knock out mice display an elevated SC pH, again demonstrating that PL hydrolysis to FFA accounts for ca.1 unit of SC pH.

Deimination of amino acid constituents of filaggrin (FLG) generates abundant polycarboxylic acids (PCA) that contribute to SC pH, demonstrated by the ca. ½ pH unit increase in pH in FLG-deficient (non-inflammatory) ichthyosis vulgaris. Within the FLG PCA mechanism, the generation of trans-urocanic acid (tUCA) from histidine, which comprises about 10% of FLG, is a major contributor to pH. However, histidase-deficient (Peruvian) mice do not display a defect in SC acidity, because they upregulate NHE1 in a compensatory fashion. Finally, the pH of darkly-pigmented human epidermis is ca. ½ pH unit lower (50-fold more acidic) than the SC of lightly-pigmented skin, due in part to the persistence and extrusion of melanin granules at the SG-SC interface. The acidic pH of darkly-pigmented skin dictates the superior function in such individuals, because acidification of lightly-pigmented human skin “resets” functions to darkly-pigmented levels. But it is likely that one additional mechanism contributes to SC acidification. Cholesterol sulfate (CSO₄) is the most abundant and widely distributed of sulfated sterols, and a critical regulator of epidermal differentiation. The CSO₄ content of the epidermis climbs to ca. 5% of lipid mass in the SG, due to enhanced expression of the gene that encodes SULT2B1b, the enzyme that sulfonates cholesterol. The hydrolytic enzyme, steroid sulfatase (SSase), then hydrolyzes CSO₄ until its levels decline to ca. 1% in the outer S. In X-linked ichthyosis (XLI), CSO₄ levels increase to ≧10% of lipid mass, and the SC in XLI is more acidic than normal, consistent with the low pKa (3.1) of CSO₄. While CSO₄ ionization would generate H₂SO₄ in situ, the ongoing hydrolysis of CSO₄ to Chol+SO₄⁼ across normal SC could also form H₂SO₄ in aqueous compartments, perhaps contributing to the selective reduction of pH that persists within extracellular domains at all levels of S.

Functions of the Acid Mantle.

In addition to antimicrobial defense, pH regulates at least three key additional functions of normal skin (Table 1). First, it is critical for epidermal permeability barrier homeostasis, because the hydrolysis of sphingomyelin and glucosylceramides into ceramides is dependent upon aSMase and β-GlcCer'ase, which display pH optima of ca. 5. Only at the reduced acidity of normal SC can these enzymes generate enough Cer necessary to form lamellar bilayers. Conversely, when the pH of the SC rises with inflammation or under experimental conditions, the activities of these enzymes decline in parallel with a deterioration of permeability barrier function.

A second cohort of pH-linked functions includes SC integrity (=resistance to stripping), SC cohesion (=protein removed per stripping), and desquamation. At the low pH of normal SC, corneodesmosomes (CD) are slowly, but progressively degraded by serine proteases (kallikreins, KLK), and then more avidly by aspartate and thiol proteases that exhibit acidic pH optima. Conversely, elevations in pH activate KLK, which rapidly degrade CD, accelerating desquamation and compromising Sc integrity/cohesion.

A third pH-linked function is pro-inflammatory cytokine activation. Large pre-formed pools of the 33 kDa precursors of pro-IL-1α and pro-IL-1β are stored in the corneocyte cytosol. As the pH of the SC rises with barrier perturbations and in inflammation, KLK activity increases, releasing the active 17 kDa forms of IL-1α/IL-1β, which in turn, initiate divergent, downstream cytokine cascades. While repeated insults lead to inflammation, single perturbations instead unleash beneficial, cytokine-initiated, homeostatic responses (e.g., accelerated DNA and lipid synthesis) that help to restore barrier homeostasis.

Developmental Variations in pH.

Full-term neonatal skin exhibits a near-neutral surface pH, with a permeability barrier which, though sufficient for terrestrial life under basal conditions, recovers more slowly than adult skin from acute perturbations. Moreover, adjustment of SC with acidic buffers normalizes barrier function in neonatal rats, and topical PPARα, PPARβ/δ or LXR activators normalize SC pH and epidermal function by increasing sPLA2 activity. Neonatal skin also displays a well-known propensity to blister, likely due to poor SC cohesion, and impaired antimicrobial defense (both pH-dependent functions). Moderately-aged skin (>55 yrs) also suffers from a flawed permeability barrier and impaired SC integrity, in parallel with an elevated SC pH, due to decreased NHE1 expression. Again, acidifying the SC normalizes both permeability barrier function and SC integrity/cohesion in moderately-aged mice.

Pathophysiology of SC pH-Inflammatory Dermatoses.

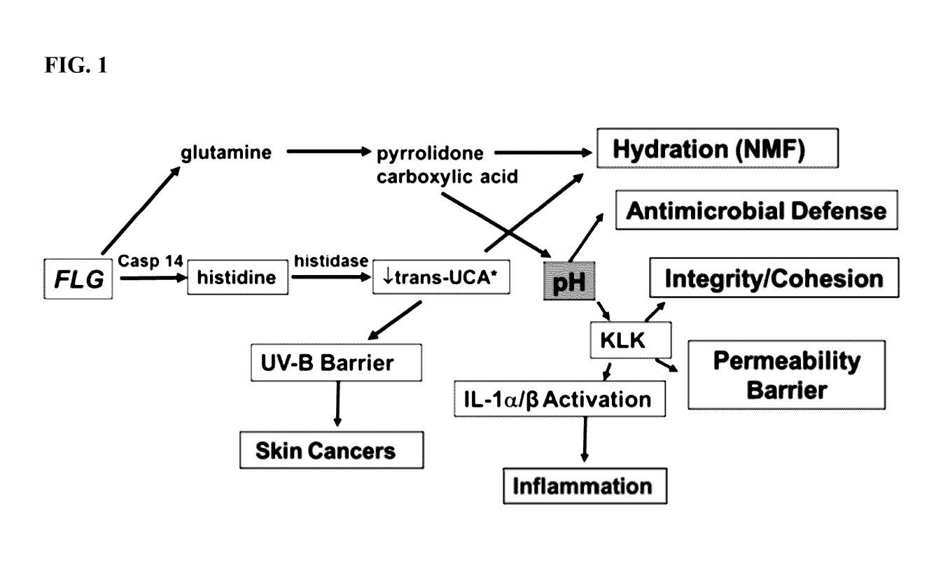

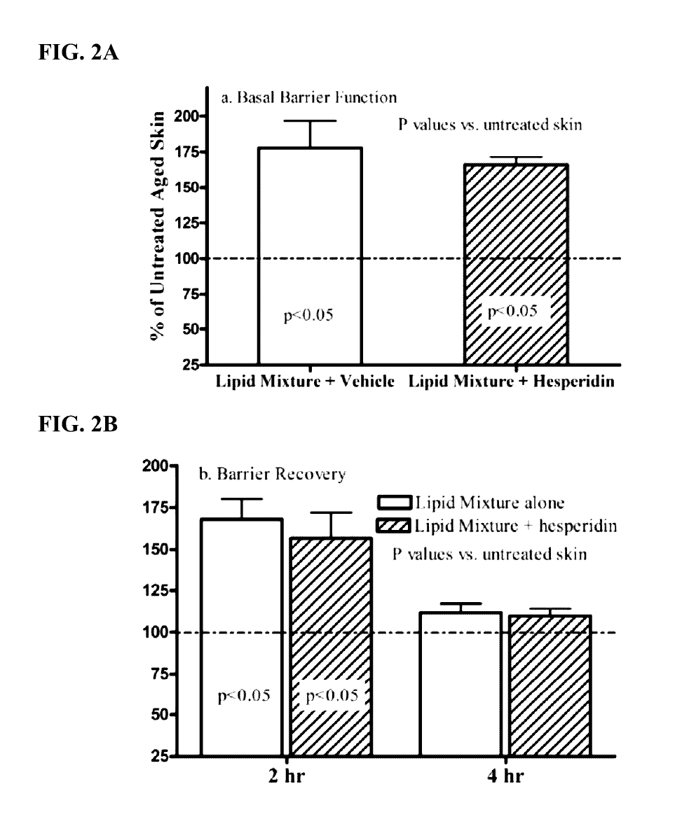

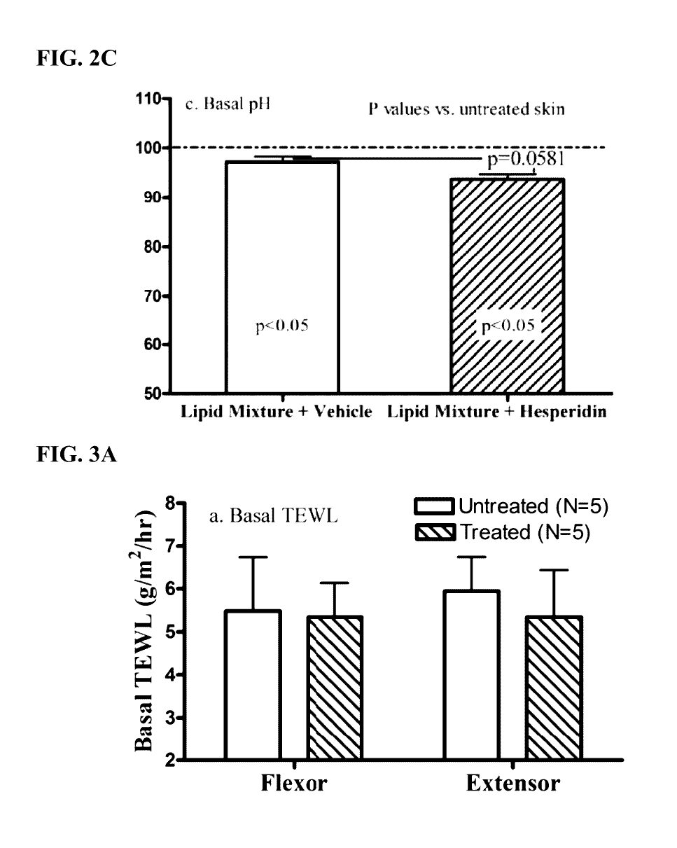

The surface pH of inflamed skin inevitably climbs towards neutrality, an increase which has direct consequences for the pathogenesis of atopic dermatitis (AD) through increased kallikreins (KLK) activity (FIG. 1). It has been shown that repeated topical applications of the universal hapten, oxazolone, produces an AD-like dermatoses (Ox-AD) in hairless mice in parallel with emergence of an elevated SC pH and a progressive barrier abnormality. It has also been shown that additional topical and aerosol applications of dust mite antigens further aggravate the Ox-AD dermatoses, ultimately yielding the next temporal component of the atopic diathesis; i.e., asthma. Importantly, maintenance of an acidic SC pH not only prevents the emergence of AD, but also the later, asthmatic component of the ‘atopic march.’ Notably, these studies teach us that AD and the atopic march can develop with repeated insults that compromise barrier function, even against a normal genetic background. It seems highly likely that pH plays a key role in the pathogenesis not only of AD, but likely also in other inflammatory dermatoses.

With regard to the antimicrobial characteristics of the inventive compositions, both the low pH and the free fatty acids in the formulation inhibit the growth of microbial pathogens, such as S. aureus, and repair of the barrier also provides a formidable barrier to the penetration of pathogens. We have also shown that applications of the triple lipid mixture enhance the production and secretion of antimicrobial peptides, such as the cathelicidin, LL-37, and human beta-defensin 2.

As discussed above, epidermal cholesterol biosynthesis has been shown to be essential for maintaining the cutaneous barrier function. One known side effect of oral statin and hypolipodemic agents is dry skin, or acquired ichthyosis resulting from lowered dermal cholesterol concentrations. For example, lovastatin, an inhibitor of cholesterol biosynthesis, produces a barrier defect when applied to normal epidermis. After lovastatin is applied, cholesterol synthesis rapidly normalizes, but fatty acid synthesis remains elevated. This indicates that a disturbance in the new fatty acid:cholesterol molar ratio accounts for the perturbed barrier function. The statin-induced alteration of the permeability of the epidermal barrier can manifest as excessive scale, cheilitis, eczemas, or acquired ichthyosis.

Additionally we treated skin of the left ventral and dorsal forearms of 5 normal, moderately-aged human subjects twice-daily with 2% hesperidin formulation (plus a 1.5% cholesterol-dominant version of the UC patent-protected barrier repair technology) in a standard oil-in-water vehicle. The ventral (flexor) surface represents chronologically-aged (CA) skin with little photoaging (PA), while the extensor surface reflects substantial PA, superimposed on CA. All 5 of the normal subjects exhibited pigment type II skin (Fitzpatrick scale of I [=redheads, always burn] to VI [=very darkly-pigmented skin, never burns, comparable to Bushmen of Sub-Saharan Africa]).

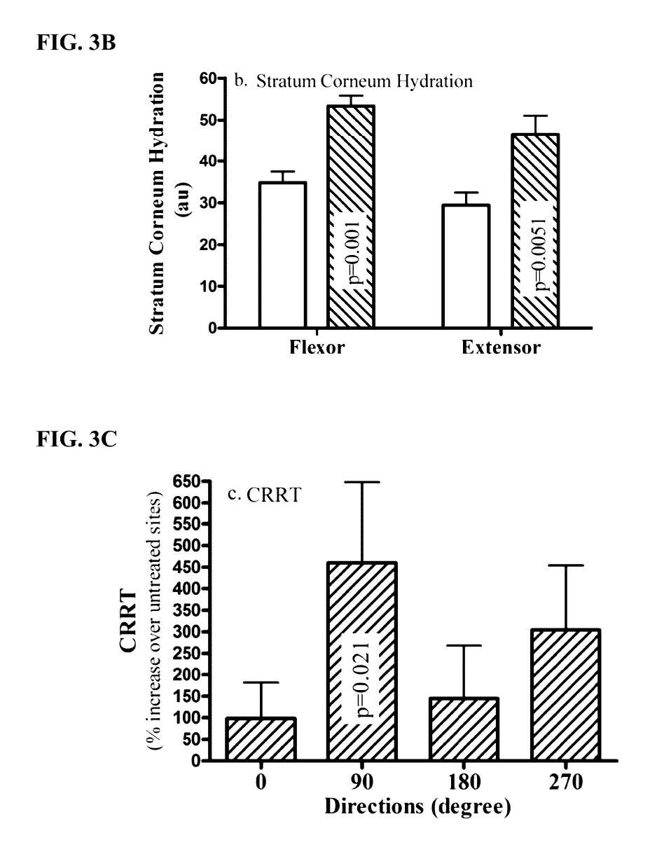

The visual results, as reported in FIGS. 3A-3C, on both chronologically (CA) and photoaged (PA) skin were striking—signs of both CA and PA, such as xerotic scaling, atrophy (skin thinning) and ecchymoses, largely disappeared in sites treated with the hesperidin-containing, barrier repair formulation. These grossly apparent changes were paralleled by marked improvements in epidermal function, including a striking improvement in stratum corneum hydration (FIG. 3B), as well as moderate benefits for basal barrier function (FIG. 3A; p<0.1). But most striking were the benefits for PA skin, reflected by changes in cutaneous resonance running time (CRRT) (FIG. 3C; p<0.02). These last results strongly suggest that the hesperidin-containing formulation penetrates through the epidermis, improving the organization of collagen fibers and other structural elements in PA human skin.

Experimental Protocols and Functional Studies.

All animal procedures were approved by the Animal Studies Subcommittee (IACUC) of the San Francisco Veterans Administration Medical Center and performed in accordance with their guidelines. Since hesperidin is not soluble in 100% ethanol, 70% ethanol was used as vehicle. Since the turnover time for hairless is about 9.5 days in normal young mice, we chose to treat aged mice for 9 days. Both flanks of 12-15 month old mice were treated topically with 60 μl of 2% hesperidin or 70% ethanol twice daily for 9 days. Basal epidermal permeability barrier function was assessed by measuring transepidermal water loss (TEWL) using TM300 connected to MPA5 (C&K, Cologne, Germany). For barrier recovery, TEWL was measured using an electrolytic water analyzer (Meeco, Warrington, Pa.) at 0, 2 and 4 hours after tape stripping (10-fold increase in TEWL), and percent barrier recovery was calculated.

Keratinocyte Culture.

Second-passage keratinocytes isolated from adult human (donor aged 60-65 year old) were cultured in serum-free keratinocyte growth medium containing 0.07 mM calcium (Clonetics, San Diego, Calif.). Cells at 60%-70% confluence were switched to a medium containing 1.2 mM calcium and treated with either 0.02% hesperidin or vehicle alone (0.02% ethanol). After 24 and 48 hrs of treatment, keratinocytes were collected for Q-PCR analysis.

Immunohistochemistry.

Immunohistochemical staining for assessing changes in epidermal differentiation was performed as follows. Briefly, 5 μm paraffin sections were incubated with the primary antibodies (Covance, Emeryville, Calif.) overnight at 4° C. After washes×3, sections were incubated with the secondary antibody for 30 minutes. Staining was detected with ABC-peroxidase kit from Vector Lab (Burlingame, Calif.). Sections were examined with a Zeiss fluorescence microscope (Jena, Germany) and digital images were captured with AxioVision software (Carl Zeiss Vision, Munich, Germany).

Q-PCR for mRNA Expression.

Total RNA was isolated from cultured human keratinocytes using TRI Reagent (Sigma). First strand cDNA was synthesized from 1 ug of total RNA with the iScript cDNA Synthesis Kit (Bio-Rad, Hercules, Calif.). The real-time PCR contained 20 ng of reversed transcribed total RNA, 450 nM forward and reverse primers, and 10 μl of 2× LightCycler 480 SYBR Green I Master in a final volume of 20 μl in 96-well plates using Mx3000PTM Real-time PCR System (Stratagene, La Jolla, Calif.). Quantification was performed by the comparative C_{T} method with 36B4 or Cyclophilin A used for normalization. The primers for lipid synthetic enzymes such as 3-hydroxy-3-methyl-glutaryl-CoA reductase (HMGCoA), serine-palmitoyl transferase 1 (SPT1), fatty acid synthase (FAS), lipid transporters (ATP-binding cassette A12 (ABCA12)), mouse beta defensin 3 (mBD3), sodium-hydrogen exchanger 1 (NHE1), secretary phospholipase A2g2f (sPLA2g2f), filaggrin, involucrin, loricrin and Cyclophilin A are listed in Primer Table 2. Relative expression of the mRNAs compared to control mRNA was calculated. Data are expressed as percentage of control (as 100%).

Electron Microscopy.

Skin biopsies from both vehicle and hesperidin-treated mice were taken for electron microscopy. Briefly, samples were minced to <0.5 mm³, fixed in modified Karnovsky's fixative overnight, and post-fixed in either 0.2% ruthenium tetroxide or 1% aqueous osmium tetroxide, containing 1.5% potassium ferrocyanide. After fixation, all samples were dehydrated in a graded ethanol series, and embedded in an Epon-epoxy mixture. Ultrathin sections were examined, with or without further contrasting with lead citrate, in a Zeiss 10A electron microscope (Carl Zeiss, Thornwood, N.J.), operated at 60 kV.

Measurement of Lamellar Body Density and Secretion.

LB numbers were determined in granular cells two to three layers below the stratum granulosum-stratum corneum (SG-SC) junction as previously described. The number of LBs was counted at 4800 magnification using a calibrated grid. Total 10 random pictures from each biopsy sample were assessed. For quantification of LB secretion, number of LB protrusion at the SG-SC junction were measured at a magnification of 5800 and correlated with the length of the bottom surface of the first SC layer on 10 random images at 5800 magnification.

Statistics.

Data are expressed as the mean+SEM. GraphPad Prism 4 software (San Diego, Calif., USA) was used for all statistical analyses. Unpaired two-tailed student's t-test with Welch's correction was used to determine the statistical significances when two groups were compared. One-Way ANOVA with Tukey correction was used when three or more groups were compared.

PRIMER TABLE 2

| | | | |
|---|---|---|---|
| Mouse HMGCoA | Forward | 5′ GCCGTGAACTGGGTCGA 3′ | SEQ ID NO: 1 |
| | Reverse | 5′ GCATATATAGCAATGTCTCCTGC 3′ | SEQ ID NO: 2 |
| Mouse SPT1 | Forward | 5′ AGGGTTCTATGGCACATTTGATGT 3′ | SEQ ID NO: 3 |
| | Reverse | 5′ TGGCTTCTTCGGTCTTCATAAAC 3′ | SEQ ID NO: 4 |
| Mouse FAS | Forward | 5′ GCTGCGGAAACTTCAGGAAAT 3′ | SEQ ID NO: 5 |
| | Reverse | 5′ AGAGACGTGTCACTCCTGGACTT 3′ | SEQ ID NO: 6 |
| h/m ABCA12 | Forward | 5′ ACAGGAATGGCCTTCATCAC 3′ | SEQ ID NO: 7 |
| | Reverse | 5′ AACATGGTGCCCTGAGAAAC 3′ | SEQ ID NO: 8 |
| Human Filaggrin | Forward | 5′ CCATCATGGATCTGCGTGG 3′ | SEQ ID NO: 9 |
| | Reverse | 5′ CACGAGAGGAAGTCTCTGCGT 3′ | SEQ ID NO: 10 |
| Mouse Filaggrin | Forward | 5′ TGACAGCCAAGTCCATTCTG 3′ | SEQ ID NO: 11 |
| | Reverse | 5′ TATCCTCCCTGACCACTTGC 3′ | SEQ ID NO: 12 |
| Human Involucrin | Forward | 5′ CTGCCTGAGCAAGAATGTGA 3′ | SEQ ID NO: 13 |
| | Reverse | 5′ TGCTCTGGGTTTTCTGCTTT 3′ | SEQ ID NO: 14 |
| Mouse Involucrin | Forward | 5′ AAGGGCTTTCCCAAACATGA 3′ | SEQ ID NO: 15 |
| | Reverse | 5′ TGCTGGTGCTCACACTTTTGA 3′ | SEQ ID NO: 16 |
| Mouse Loricrin | Forward | 5′ GTGGAAAGACCTCTGGTGGA 3′ | SEQ ID NO: 17 |
| | Reverse | 5′ TGGAACCACCTCCATAGGAA 3′ | SEQ ID NO: 18 |
| Human BD3 | Forward | 5′ CCTAGCAGCTATGAGGATCCAT 3′ | SEQ ID NO: 19 |
| | Reverse | 5′ CTTCGGCAGCATTTTCG 3′ | SEQ ID NO: 20 |
| Mouse BD3 | Forward | 5′ TCTGTTTGCATTTCTCCTGGT 3′ | SEQ ID NO: 21 |
| | Reverse | 5′ GGAACTCCACAACTGCCAAT 3′ | SEQ ID NO: 22 |
| Human CAMP | Forward | 5′ GCTAACCTCTACCGCCTCCT 3′ | SEQ ID NO: 23 |
| | Reverse | 5′ GGTCACTGTCCCCATACACC 3′ | SEQ ID NO: 24 |
| Mouse CAMP | Forward | 5′ TGAGCCCCAAGGGGACGAGG 3′ | SEQ ID NO: 25 |
| | Reverse | 5′ GCCGGGTTCAGGGTGACTGC 3′ | SEQ ID NO: 26 |
| Human CycloA | Forward | 5′ TCTCCTTTGAGCTGTTTGCAG 3′ | SEQ ID NO: 27 |
| | Reverse | 5′ CACCACATGCTTGCCATC 3′ | SEQ ID NO: 28 |
| h/m 36B4 | Forward | 5′ GCGACCTGGAAGTCCAACTAC 3′ | SEQ ID NO: 29 |
| | Reverse | 5′ ATCTGCTGCATCTGCTTGG 3′ | SEQ ID NO: 30 |
| Mouse NHE1 | Forward | 5′-TTTCCCCGATTTCCTTCTCT-3′ | SEQ ID NO: 31 |
| | Reverse | 5′-GCGTGTAAGACCTGGGACAT-3′ | SEQ ID NO: 32 |
| Mouse sPLA2g2f | Forward | 5′-CCCCATCCAGTCCTTAGTCA-3′ | SEQ ID NO: 33 |
| | Reverse | 5′-ACTTCTGGGCAGGAGTCAGA-3′ | SEQ ID NO: 34 |
| Human GAPDH | Forward | 5′CGAGTCAACGGATTTGGTCGTA3′ | SEQ ID NO: 35 |
| | Reverse | 5′GCAACAATATCCACTTTACCAGAGTTAA3′ | SEQ ID NO: 36 |

### Example of a Topical Composition

| | % Range By | |
|---|---|---|
| Raw Material | Weight | Ingredient Function |
| Cholesterol | 1.0 | Barrier Lipid |
| Dimethicone | 1.83 | Skin Conditioner |
| Disodium EDTA | 0.10 | Chelating Agent |
| Glycerin | 10% | Humectant |
| Hydroxypropyl Bispalmitamide | 1.0 | Barrier Lipid |
| MEA (Ceramide PC-104) | | |
| Conjugated Linoleic Acid | 0.25 | Barrier Lipid |
| Glyceryl Stearate PEG-100 | 1.59 | Emulsifier |
| Petrolatum | 2.00 | Emollient |
| Phenoxyethanol | 0.70 | Preservative |
| Squalane | 2.5 | Emollient |
| Citric Acid | ≧0.5 | Buffer |
| | (to reduce | |
| | pH to 3-5) | |
| Sorbic Acid | 0.10 | Preservative |
| Corn Syrup Solids (A blend of | 7.02 | Encapsulation Aid |
| HI-CAP 10 and HI-CAP 20) | | |
| Euphorbia Cerifera (Candelilla) | 0.78 | Encapsulation Aid |
| Wax | | |
| Xanthan Gum | 0.22 | Aqueous phase thickener |
| Purified USO Grade Water | QS to 100 | Diluent |
| Hesperidin | 2.0 | Anti-aging |

The lipids may be diluted to determine whether the impact of hesperidin could be separated out, and whether a potential cost saving could be achieved by lowering the amount of ceramide needed. It was observed that the dilute mixture does very well for improving barrier function (accelerates barrier recovery and lowers basal TEWL levels). It was noted that hesperidin significantly improved (lowered) pH when added to the triple lipid mixture.

Example preparation of a topical composition: The active ingredients are solubilized in preparation for topical application in organic solvents suitable for dermal application, such as ethanol, aqueous ethanol, acetone, aqueous acetone, or combinations of acetone and ethanol, with or without water. Mixtures of propylene glycol and ethanol or propylene glycol and aqueous ethanol are also suitable. Alternatively, mild detergents, such as MIRANOL®, can be used to solubilize the mixture of active ingredients.

The composition includes a cholesterol-dominant lipid mixture plus hesperidin for the use on aged mice: 18-Month old mice were divided into groups of untreated, lipid mixture+vehicle (70% ethanol) treated, and lipid mixture+2% hesperidin treated. Mice are treated twice daily for 7 days. All measurements are carried out with a MPA5 physiology monitor. Lipid mixture contained cholesterol, fatty acid and ceramide (3:1:1 molar ratio, with cholesterol at 3 moles) at a concentration of 0.74 wt %. Data are normalized to untreated control, setting untreated as 100%.

The specific examples disclosed above are to be construed as merely illustrative, and not limitative of the remainder of the disclosure in any way whatsoever. Without further elaboration, it is believed that one skilled in the art can, based on the description herein, utilize the present invention to its fullest extent.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the present claims.

All publications cited herein are hereby incorporated by reference in their entirety.

### EMBODIMENTS

### Embodiment P1

A topical composition for treating dry skin or skin suffering an abnormal epidermal barrier function, said composition comprising:
a) about 1 to about 50 wt % of cholesterol;
b) about 0.01 to about 5 wt % of an acid buffer; and
c) about 0.001 to about 50 wt % of hesperidin;
   wherein the pH of said topical composition is about 3 to about 5.5.

### Embodiment P2

A topical composition for treating aged skin, said composition comprising:
a) a mixture of barrier lipids selected from the group consisting of cholesterol, at least one ceramide, at least one free fatty acid, and mixtures of two or more thereof, in a total of about 1 to about 50 wt %;
b) about 0.1 to about 10 wt % of a skin conditioner;
c) about 0.001 to about 5 wt % of a chelating agent;
d) about 1 to about 30 wt % of a humectant;
e) about 0.5 to about 20 wt % of an emulsifier;
f) one or more emollients in a total of about 1 to about 30 wt %;
g) one or more preservatives in a total of about 0.1 to about 10 wt %;
h) about 0.01 to about 5 wt % of an acid buffer;
i) one or more encapsulation aids in a total of about 1 to about 25 wt %;
j) about 0.01 to about 3 wt % of an aqueous phase thickener; and
k) about 0.001 to about 50 wt % of hesperidin.

### Embodiment P3

The composition of Embodiment P2, wherein said mixture of barrier lipids consists of cholesterol, hydroxypropyl bispalmitamide MEA (Ceramide PC-104) and conjugated linoleic acid (CLA).

### Embodiment P4

The composition of Embodiment P2 or P3, wherein said skin conditioner comprises dimethicone, said chelating agent comprises disodium EDTA, said humectant comprises glycerin, and said acid buffer comprises citric acid.

### Embodiment P5

The composition of any one of Embodiments P2 to P4, wherein said emulsifier comprises glyceryl stearate PEG-100.

### Embodiment P6

The composition of any one of Embodiments P2 to P5, wherein said one or more emollients comprise petrolatum and squalane.

### Embodiment P7

The composition of any one of Embodiments P2 to P6, wherein said one or more preservatives comprise phenoxyethanol and sorbic acid.

### Embodiment P8

The composition of any one of Embodiments P2 to P7, wherein said one or more encapsulation aids comprise corn syrup solids and euphorbia cerifera (candelilla) wax.

### Embodiment P9

The composition of any one of Embodiments P2 to P8, wherein said aqueous phase thickener comprises xanthan gum.

### Embodiment P10

The composition of any one of Embodiments P2 to P9, wherein the pH of said topical composition is about 3 to about 5.5.

### Embodiment P11

The composition of any one of Embodiments P2 to P10, wherein said molar ratio of cholesterol:ceramide:free fatty acid is 3:1:1.

### Embodiment P12

The composition of any one of Embodiments P2 to P11, wherein cholesterol is present in about 0.1 to about 10 wt %.

### Embodiment P13

The composition of any one of Embodiments P2 to P12, wherein said ceramide is present in about 0.1 to about 10 wt %.

### Embodiment P14

The composition of any one of Embodiments P2 to P13, wherein said free fatty acid is present in about 0.01 to about 6 wt %.

### Embodiment P15

The topical composition for treating aged skin of Embodiment P2, said composition consisting of:
a) about 1 to about 50 wt % of a mixture of barrier lipids consisting of cholesterol, one or more ceramides, and one or more free fatty acids;
b) about 0.1 to about 10 wt % of a skin conditioner;
c) about 0.001 to about 5 wt % of a chelating agent;
d) about 1 to about 30 wt % of a humectant;
e) about 0.5 to about 20 wt % of glyceryl stearate PEG-100 emulsifier;
f) about 1 to about 30 wt % of a mixture of emollients consisting of petrolatum and squalane;
g) about 0.1 to about 10 wt % of a mixture of preservatives consisting of phenoxyethanol and sorbic acid;
h) about 0.01 to about 5 wt % of citric acid;
i) about 1 to about 25 wt % of a mixture of encapsulation aids consisting of corn syrup solids and euphorbia cerifera (candelilla) wax;
j) about 0.01 to about 3 wt % of an aqueous phase thickener;
k) about 0.001 to about 50 wt % of hesperidin; and
l) water.

### Embodiment P16

An anti-inflammatory composition comprising:
a) a mixture of barrier lipids selected from the group consisting of cholesterol, ceramides, free fatty acids and mixtures of two or more thereof, in a total of about 1 to about 50 wt %;
b) about 0.001 to about 50 wt % of hesperidin;
c) about 0.01 to about 5 wt % of an acid buffer; and
d) about 0.001 to about 5% of at least one glucocorticoid.

### Embodiment P17

A method of treating aged skin, comprising administering said topical composition of any one of Embodiments P1 to P16 to at least a portion of aged skin, in an amount effective to relieve or reverse clinical indications of skin aging.

### Embodiment P18

The method of Embodiment P17, wherein the clinical indications of skin aging are selected from the group consisting of eczema, xerosis, xerotic eczema, winter itch, nummular eczema, seborrheic dermatitis, occupational dermatitis, hand eczema, and stasis dermatitis.

### Embodiment P19

A method of preventing or reversing dermal side effects of systemic or topical glucocorticoid treatment, comprising treating at least a portion of affected skin with said topical composition of any one of Embodiments P1 to P16, in an amount effective to prevent or reverse dermal clinical indications of glucocorticoid treatment.

### Embodiment P20

The method of Embodiment P19, wherein said dermal clinical indications of glucocorticoid treatment are selected from the group consisting of inflammation, tachyphylaxis, disease flares, and rebound flares.

### Embodiment P21

A method of treating inflammation comprising administering an effective amount of the anti-inflammatory composition of Embodiment P16 to at least a portion of affected skin in need thereof.

### Embodiment P22

A method for the treatment of dry skin and/or abnormal epidermal barrier function associated with oral hypolipodemic agent treatment, comprising administering said topical composition of Embodiment P1 or P11 to at least a portion of affected skin of a subject, in an amount effective to relieve or reverse clinical indications of hypolipodemic treatment.

### Embodiment P23

The method of Embodiment P22, wherein the subject has taken a hypolipodemic agent or is expected to receive such treatment.

### Embodiment P24

A composition comprising cholesterol and hesperidin, wherein:
(i) said composition further comprises a ceramide and a free fatty acid wherein the mole ratio of total cholesterol:total ceramide:total free fatty acid is about 3:1:1 moles; or
(ii) the pH of the composition is between about 3 to about 5.5.

### Embodiment P25

The composition of Embodiment P24, comprising about 1 to about 50 wt % of total cholesterol.

### Embodiment P26

The composition of one of Embodiments P24 to P25 comprising about 0.001 to about 50 wt % of hesperidin.

### Embodiment P27

The composition of one of Embodiments P25 to P26, comprising about 0.1 to about 10 wt % total ceramide.

### Embodiment 28

The composition of one of Embodiments P25 to P27, comprising about 0.01 to about 6 wt % total free fatty acid.

### Embodiment P29

The composition of one of Embodiments P24 to P28, comprising about 0.01 to about 5 wt % of acid buffer.

### Embodiment P30

The composition of one of Embodiments P24 to P29, further comprising a skin conditioner.

### Embodiment P31

The composition of Embodiment P30, comprising about 0.1 to about 10 wt % of the skin conditioner.

### Embodiment P32

The composition of one of Embodiments P30 to P31, wherein the skin conditioner is dimethicone.

### Embodiment P33

The composition of one of Embodiments P24 to P32, further comprising a chelating agent.

### Embodiment P34

The composition of Embodiment P33, comprising about 0.001 to about 5 wt % of the chelating agent.

### Embodiment P35

The composition of one of Embodiments P33 to P34, wherein the chelating agent is disodium EDTA.

### Embodiment P36

The composition of one of Embodiments P24 to P35, further comprising a humectant.

### Embodiment P37

The composition of Embodiment P36, comprising about 1 to about 30 wt % of the humectant.

### Embodiment P38

The composition of one of Embodiments P36 to P37, wherein the humectant is glycerin.

### Embodiment P39

The composition of one of Embodiments P24 to P38, further comprising an emulsifier.

### Embodiment P40

The composition of Embodiment P39, comprising about 0.5 to about 20 wt % of the emulsifier.

### Embodiment P41

The composition of one of Embodiments P38 to P39, wherein the emulsifier is glyceryl stearate PEG-100.

### Embodiment P42

The composition of one of Embodiments P24 to P41, further comprising an emollient.

### Embodiment P43

The composition of Embodiment P42, comprising about 1 to about 30 wt % of the emollient.

### Embodiment P44

The composition of one of Embodiments P42 to P43, wherein the emollient is petrolatum.

### Embodiment P45

The composition of one of Embodiments P42 to P43, wherein the emollient is squalane.

### Embodiment P46

The composition of one of Embodiments P24 to P45, further comprising a preservative.

### Embodiment P47

The composition of Embodiment P46, comprising about 0.1 to about 10 wt % of the preservative.

### Embodiment P48

The composition of one of Embodiments P46 to P47, wherein the preservative is phenoxyethanol.

### Embodiment P49

The composition of one of Embodiments P46 to P47, wherein the preservative is sorbic acid.

### Embodiment P50

The composition of one of Embodiments P24 to P49, further comprising an encapsulation aid.

### Embodiment P51

The composition of Embodiment P50, comprising about 1 to about 25 wt % of the encapsulation aid.

### Embodiment P52

The composition of one of Embodiments P50 to P51, wherein the encapsulation aid is euphorbia cerifera (candelilla) wax.

### Embodiment P53

The composition of one of Embodiments P50 to P51, wherein the encapsulation aid is corn syrup solids.

### Embodiment P54

The composition of one of Embodiments P24 to P53, further comprising an aqueous phase thickener.

### Embodiment P55

The composition of Embodiment P54, comprising about 0.01 to about 3 wt % of the aqueous phase thickener.

### Embodiment P56

The composition of one of Embodiments P54 to P55, wherein the aqueous phase thickener is xanthan gum.

### Embodiment P57

The composition of one of Embodiments P24 to P56, wherein the ceramide is hydroxypropyl bispalmitamide MEA (Ceramide PC-104).

### Embodiment P58

The composition of one of Embodiments P24 to P57, wherein the free fatty acid is conjugated linoleic acid (CLA).

### Embodiment P59

The composition of one of Embodiments P24 to P58, further comprising a glucocorticoid.

### Embodiment P60

The composition of Embodiment P59, comprising about 0.001 to about 5 wt % of the glucocorticoid.

### Embodiment P61

The composition of one of Embodiments P24 to P60, wherein the composition is a topical composition.

### Embodiment P62

The composition of one of Embodiments P24 to P61 for use in treating dry skin in a subject in need thereof.

### Embodiment P63

The composition of one of Embodiments P24 to P61 for use in treating abnormal epidermal barrier function in a subject in need thereof.

### Embodiment P64

The composition of one of Embodiments P24 to P61 for use in treating an inflammatory condition in a subject in need thereof.

### Embodiment P65

The composition of one of Embodiments P24 to P61 for use in treating a clinical indication of skin aging in a subject in need thereof.

### Embodiment P66

The composition of Embodiment P65, wherein the clinical indication of skin aging is eczema, xerosis, xerotic eczema, winter itch, nummular eczema, seborrheic dermatitis, occupational dermatitis, hand eczema, or stasis dermatitis.

### Embodiment P67

The composition of one of Embodiments P24 to P61 for use in treating a dermal clinical indication of glucocorticoid treatment in a subject in need thereof.

### Embodiment P68

The composition of Embodiment P67, wherein the dermal clinical indication of glucocorticoid treatment is inflammation, tachyphylaxis, disease flares, or rebound flares.

### Embodiment P69

The composition of one of Embodiments P24 to P61 for use in treating a clinical indication of hypolipodemic treatment in a subject in need thereof.

### Embodiment U1

A topical composition, said composition comprising:

a) about 1 to about 50 wt % of cholesterol;
b) about 0.01 to about 5 wt % of an acid buffer; and
c) about 0.001 to about 50 wt % of hesperidin;
wherein the pH of said topical composition is from about 3 to about 5.5.

### Embodiment U2

A topical composition, said composition comprising:

a) a mixture of barrier lipids selected from the group consisting of cholesterol, at least one ceramide, at least one free fatty acid, and mixtures of two or more thereof, in a total of about 1 to about 50 wt %;
b) about 0.1 to about 10 wt % of a skin conditioner;
c) about 0.001 to about 5 wt % of a chelating agent;
d) about 1 to about 30 wt % of a humectant;
e) about 0.5 to about 20 wt % of an emulsifier;
f) one or more emollients in a total of about 1 to about 30 wt %;
g) one or more preservatives in a total of about 0.1 to about 10 wt %;
h) about 0.01 to about 5 wt % of an acid buffer;
i) one or more encapsulation aids in a total of about 1 to about 25 wt %;
j) about 0.01 to about 3 wt % of an aqueous phase thickener; and
k) about 0.001 to about 50 wt % of hesperidin.

### Embodiment U3

A topical composition, said composition comprising: a mixture of barrier lipids selected from the group consisting of cholesterol, at least one ceramide, at least one free fatty acid, and mixtures of two or more thereof.

### Embodiment U4

The composition of Embodiments U3, wherein the composition further comprises a skin conditioner.

### Embodiment U5

The composition of any one of Embodiments U3 to U4, wherein the composition further comprises a chelating agent.

### Embodiment U6

The composition of any one of Embodiments U3 to U5, wherein the composition further comprises about 1 to about 30 wt % of a humectant.

### Embodiment U7

The composition of any one of Embodiments U3 to U6, wherein the composition further comprises an emulsifier.

### Embodiment U8

The composition of any one of Embodiments U3 to U7, wherein the composition further comprises one or more emollients.

### Embodiment U9

The composition of any one of Embodiments U3 to U8, wherein the composition further comprises one or more preservatives.

### Embodiment U10

The composition of any one of Embodiments U3 to U9, wherein the composition further comprises an acid buffer.

### Embodiment U11

The composition of any one of Embodiments U3 to U10, wherein the composition further comprises one or more encapsulation aids.

### Embodiment U12

The composition of any one of Embodiments U3 to U11, wherein the composition further comprises an aqueous phase thickener.

### Embodiment U13

The composition of any one of Embodiments U3 to U12, wherein the composition further comprises hesperidin.

### Embodiment U14

A topical composition, said composition comprising: a mixture of barrier lipids selected from the group consisting of cholesterol, at least one ceramide, at least one free fatty acid, and mixtures of two or more thereof, in a total of about 1 to about 50 wt %.

### Embodiment U15

The composition of U14, wherein the composition further comprises about 0.1 to about 10 wt % of a skin conditioner.

### Embodiment U16

The composition of any one of Embodiments U14 to U15, wherein the composition further comprises about 0.001 to about 5 wt % of a chelating agent.

### Embodiment U17

The composition of any one of Embodiments U14 to U16, wherein the composition further comprises about 1 to about 30 wt % of a humectant.

### Embodiment U18

The composition of any one of Embodiments U14 to U17, wherein the composition further comprises about 0.5 to about 20 wt % of an emulsifier.

### Embodiment U19

The composition of any one of Embodiments U14 to U18, wherein the composition further comprises one or more emollients in a total of about 1 to about 30 wt %.

### Embodiment U20

The composition of any one of Embodiments U14 to U19, wherein the composition further comprises one or more preservatives in a total of about 0.1 to about 10 wt %.

### Embodiment U21

The composition of any one of Embodiments U14 to U20, wherein the composition further comprises about 0.01 to about 5 wt % of an acid buffer.

### Embodiment U22

The composition of any one of Embodiments U14 to U21, wherein the composition further comprises one or more encapsulation aids in a total of about 1 to about 25 wt %.

### Embodiment U23

The composition of any one of Embodiments U14 to U22, wherein the composition further comprises about 0.01 to about 3 wt % of an aqueous phase thickener.

### Embodiment U24

The composition of any one of Embodiments U14 to U23, wherein the composition further comprises about 0.001 to about 50 wt % of hesperidin.

### Embodiment U25

The composition of any one of Embodiments U2 to U24, wherein said mixture of barrier lipids consists of cholesterol, hydroxypropyl bispalmitamide MEA (Ceramide PC-104) and conjugated linoleic acid (CLA); said skin conditioner comprises dimethicone; said chelating agent comprises disodium EDTA; said humectant comprises glycerin; and said acid buffer comprises citric acid; emulsifier comprises glyceryl stearate PEG-100; wherein said one or more emollients comprise petrolatum and squalane; wherein said one or more preservatives comprise phenoxyethanol and sorbic acid; wherein said one or more encapsulation aids comprise corn syrup solids and euphorbia cerifera (candelilla) wax; wherein said aqueous phase thickener comprises xanthan gum; wherein the pH of said topical composition is about 3 to about 5.5; and herein the molar ratio of cholesterol:ceramide:free fatty acid is 3:1:1.

### Embodiment U26

The composition of any one of Embodiments U2 to U25, wherein cholesterol is present in about 0.1 to about 10 wt %; wherein said ceramide is present in about 0.1 to about 10 wt %; and wherein said free fatty acid is present in about 0.01 to about 6 wt %.

### Embodiment U27

A composition comprising:

a) a mixture of barrier lipids selected from the group consisting of cholesterol, ceramides, free fatty acids and mixtures of two or more thereof, in a total of about 1 to about 50 wt %;
b) about 0.001 to about 50 wt % of hesperidin;
c) about 0.01 to about 5 wt % of an acid buffer; and
d) about 0.001 to about 5% of at least one glucocorticoid.

### Embodiment U28

A method of treating aged skin, comprising administering the topical composition of Embodiment U1 to at least a portion of aged skin, in an amount effective to relieve or reverse a clinical indication of skin aging, wherein said clinical indication of skin aging is selected from the group consisting of eczema, xerosis, xerotic eczema, winter itch, nummular eczema, seborrheic dermatitis, occupational dermatitis, hand eczema, and stasis dermatitis.

### Embodiment U29

A method of preventing or reversing dermal side effects of systemic or topical glucocorticoid treatment, comprising treating at least a portion of affected skin with the topical composition of Embodiment U1, in an amount effective to prevent or reverse dermal a clinical indication of glucocorticoid treatment, wherein said dermal clinical indication of glucocorticoid treatment is selected from the group consisting of inflammation, tachyphylaxis, disease flares, and rebound flares.

### Embodiment U30

A method of treating dry skin or abnormal epidermal barrier function associated with oral hypolipodemic agent treatment, comprising administering the topical composition of Embodiment U1 to at least a portion of affected skin of a subject, in an amount effective to relieve or reverse a clinical indication of hypolipodemic treatment.

### Embodiment U31

A composition comprising cholesterol and hesperidin, wherein:

(i) said composition further comprises a ceramide and a free fatty acid wherein the mole ratio of total cholesterol total ceramide:total free fatty acid is about 3:1:1 moles; or
(ii) the pH of the composition is from about 3 to about 5.5.

### Embodiment U32

The composition of Embodiment U31, wherein:

(i) said composition further comprises a ceramide and a free fatty acid wherein the mole ratio of total cholesterol total ceramide:total free fatty acid is about 3:1:1 moles; and
(ii) the pH of the composition is from about 3 to about 5.5.

### Embodiment U33

The composition of any one of Embodiments U31 to U32, comprising about 1 to about 50 wt % of total cholesterol; comprising about 0.1 to about 10 wt % total ceramide; comprising about 0.01 to about 6 wt % total free fatty acid; and comprising about 0.001 to about 50 wt % of hesperidin.

### Embodiment U34

The composition of any one of Embodiments U31 to U33, further comprising an acid buffer.

### Embodiment U35

The composition of any one of Embodiments U31 to U34, further comprising a skin conditioner; a humectant; an emulsifier; a chelating agent; and an emollient.

### Embodiment U36

The composition of any one of Embodiments U31 to U34, further comprising a preservative; an aqueous phase thickener; and an encapsulation aid.

### Embodiment U37

The composition of any one of Embodiments U31 to U36, further comprising a glucocorticoid.

### Embodiment U38

The composition of any one of Embodiments U31 to U37, for use in treating dry skin in a subject in need thereof.

### Embodiment U39

The composition of any one of Embodiments U31 to U38, for use in treating abnormal epidermal barrier function in a subject in need thereof.

### Embodiment U40

The composition of any one of Embodiments U31 to U39, for use in treating an inflammatory condition in a subject in need thereof.

### Embodiment U41

The composition of any one of Embodiments U31 to U39, for use in treating a clinical indication of skin aging in a subject in need thereof, wherein said clinical indication of skin aging is selected from the group consisting of eczema, xerosis, xerotic eczema, winter itch, nummular eczema, seborrheic dermatitis, occupational dermatitis, hand eczema, and stasis dermatitis.

### Embodiment U42

The composition of any one of Embodiments U31 to U39, for use in treating a dermal clinical indication of glucocorticoid treatment in a subject in need thereof, wherein said dermal clinical indication of glucocorticoid treatment is selected from the group consisting of inflammation, tachyphylaxis, disease flares, and rebound flares.

### Embodiment U43

A method for treating dry skin said method comprising administering to a subject in need thereof an effective amount of composition of any one of Embodiments U31 to U39.

### Embodiment U44

A method for treating abnormal epidermal barrier function said method comprising administering to a subject in need thereof an effective amount of composition of any one of Embodiments U31 to U39.

### Embodiment U45

A method for treating an inflammatory condition said method comprising administering to a subject in need thereof an effective amount of composition of any one of Embodiments U31 to U39.

### Embodiment U46

A method for treating a clinical indication of skin aging said method comprising administering to a subject in need thereof an effective amount of composition of any one of Embodiments U31 to U39, wherein said clinical indication of skin aging is selected from the group consisting of eczema, xerosis, xerotic eczema, winter itch, nummular eczema, seborrheic dermatitis, occupational dermatitis, hand eczema, and stasis dermatitis.

### Embodiment U47

A method for treating a dermal clinical indication of glucocorticoid treatment said method comprising administering to a subject in need thereof an effective amount of composition of any one of Embodiments U31 to U39, wherein said dermal clinical indication of glucocorticoid treatment is selected from the group consisting of inflammation, tachyphylaxis, disease flares, and rebound flares.

### ADDITIONAL EMBODIMENTS

### Embodiment 1

A topical composition, the composition comprising:

a) about 1 to about 50 wt % of cholesterol;
b) about 0.01 to about 5 wt % of an acid buffer; and
c) about 0.001 to about 50 wt % of hesperidin;
wherein the pH of the topical composition is from about 3 to about 5.5.

### Embodiment 2

The composition of Embodiment 1, wherein the composition is capable of treating dry skin or skin suffering an abnormal epidermal barrier function.

### Embodiment 3

A topical composition, the composition comprising:

a) a mixture of barrier lipids selected from the group consisting of cholesterol, at least one ceramide, at least one free fatty acid, and mixtures of two or more thereof, in a total of about 1 to about 50 wt %;
b) about 0.1 to about 10 wt % of a skin conditioner;
c) about 0.001 to about 5 wt % of a chelating agent;
d) about 1 to about 30 wt % of a humectant;
e) about 0.5 to about 20 wt % of an emulsifier;
f) one or more emollients in a total of about 1 to about 30 wt %;
g) one or more preservatives in a total of about 0.1 to about 10 wt %;
h) about 0.01 to about 5 wt % of an acid buffer;
i) one or more encapsulation aids in a total of about 1 to about 25 wt %;
j) about 0.01 to about 3 wt % of an aqueous phase thickener; and
k) about 0.001 to about 50 wt % of hesperidin.

### Embodiment 4

The composition of Embodiment 3, wherein the composition is capable of treating aged skin.

### Embodiment 5

The composition of one of Embodiment 3 or 4, wherein the mixture of barrier lipids consists of cholesterol, hydroxypropyl bispalmitamide MEA (Ceramide PC-104) and conjugated linoleic acid (CLA).

### Embodiment 6

The composition of any one of Embodiments 3 to 5, wherein the skin conditioner comprises dimethicone, the chelating agent comprises disodium EDTA, the humectant comprises glycerin, and the acid buffer comprises citric acid.

### Embodiment 7

The composition of any one of Embodiments 3 to 6, wherein the emulsifier comprises glyceryl stearate PEG-100.

### Embodiment 8

The composition of any one of Embodiments 3 to 7, wherein the one or more emollients comprise petrolatum and squalane.

### Embodiment 9

The composition of any one of Embodiments 3 to 8, wherein the one or more preservatives comprise phenoxyethanol and sorbic acid.

### Embodiment 10

The composition of any one of Embodiments 3 to 9, wherein the one or more encapsulation aids comprise corn syrup solids and euphorbia cerifera (candelilla) wax.

### Embodiment 11

The composition of any one of Embodiments 3 to 10, wherein the aqueous phase thickener comprises xanthan gum.

### Embodiment 12

The composition of any one of Embodiments 3 to 11, wherein the pH of the topical composition is from about 3 to about 5.5.

### Embodiment 13

The composition of any one of Embodiments 3 to 12, wherein the molar ratio of cholesterol:ceramide:free fatty acid is 3:1:1.

### Embodiment 14

The composition of any one of Embodiments 3 to 13, wherein cholesterol is present in about 0.1 to about 10 wt %.

### Embodiment 15

The composition of any one of Embodiments 3 to 14, wherein the ceramide is present in about 0.1 to about 10 wt %.

### Embodiment 16

The composition of any one of Embodiments 3 to 15, wherein the free fatty acid is present in about 0.01 to about 6 wt %.

### Embodiment 17

The topical composition of Embodiment 4, the composition consisting of:

a) about 1 to about 50 wt % of a mixture of barrier lipids consisting of cholesterol, one or more ceramides, and one or more free fatty acids;
b) about 0.1 to about 10 wt % of a skin conditioner;
c) about 0.001 to about 5 wt % of a chelating agent;
d) about 1 to about 30 wt % of a humectant;
e) about 0.5 to about 20 wt % of glyceryl stearate PEG-100 emulsifier;
f) about 1 to about 30 wt % of a mixture of emollients consisting of petrolatum and squalane;
g) about 0.1 to about 10 wt % of a mixture of preservatives consisting of phenoxyethanol and sorbic acid;
h) about 0.01 to about 5 wt % of citric acid;
i) about 1 to about 25 wt % of a mixture of encapsulation aids consisting of corn syrup solids and euphorbia cerifera (candelilla) wax;
j) about 0.01 to about 3 wt % of an aqueous phase thickener;
k) about 0.001 to about 50 wt % of hesperidin; and
l) water.

### Embodiment 18

A composition comprising:

a) a mixture of barrier lipids selected from the group consisting of cholesterol, ceramides, free fatty acids and mixtures of two or more thereof, in a total of about 1 to about 50 wt %;
b) about 0.001 to about 50 wt % of hesperidin;
c) about 0.01 to about 5 wt % of an acid buffer; and
d) about 0.001 to about 5% of at least one glucocorticoid.

### Embodiment 19

The composition of Embodiment 18, the composition is an anti-inflammatory composition.

### Embodiment 20

A method of treating aged skin, comprising administering the topical composition of any one of Embodiments 1 to 19 to at least a portion of aged skin, in an amount effective to relieve or reverse a clinical indication of skin aging.

### Embodiment 21

The method of Embodiment 20, wherein the clinical indication of skin aging is selected from the group consisting of eczema, xerosis, xerotic eczema, winter itch, nummular eczema, seborrheic dermatitis, occupational dermatitis, hand eczema, and stasis dermatitis.

### Embodiment 22

A method of preventing or reversing dermal side effects of systemic or topical glucocorticoid treatment, comprising treating at least a portion of affected skin with the topical composition of any one of Embodiments 1 to 19, in an amount effective to prevent or reverse a dermal clinical indication of glucocorticoid treatment.

### Embodiment 23

The method of Embodiment 22, wherein the dermal clinical indication of glucocorticoid treatment is selected from the group consisting of inflammation, tachyphylaxis, disease flares, and rebound flares.

### Embodiment 24

A method of treating inflammation comprising administering an effective amount of the composition of one of Embodiments 1 to 19 to at least a portion of affected skin in need thereof.

### Embodiment 25

A method for the treatment of dry skin and/or abnormal epidermal barrier function associated with oral hypolipodemic agent treatment, comprising administering the topical composition of Embodiment 1 or 19 to at least a portion of affected skin of a subject, in an amount effective to relieve or reverse a clinical indication of hypolipodemic treatment.

### Embodiment 26

The method of Embodiment 25, wherein the subject has been administered a hypolipodemic agent or is co-administered with a hypolipodemic agent.

### Embodiment 27

A composition comprising cholesterol and hesperidin, wherein:

(i) the composition further comprises a ceramide and a free fatty acid wherein the mole ratio of total cholesterol total ceramide:total free fatty acid is about 3:1:1 moles; or
(ii) the pH of the composition is from about 3 to about 5.5.

### Embodiment 28

The composition of Embodiment 27, wherein:

(i) the composition further comprises a ceramide and a free fatty acid wherein the mole ratio of total cholesterol total ceramide:total free fatty acid is about 3:1:1 moles; and
(ii) the pH of the composition is from about 3 to about 5.5.

### Embodiment 29

The composition of Embodiment 27, comprising about 1 to about 50 wt % of total cholesterol.

### Embodiment 30

The composition of one of Embodiments 27 to 29 comprising about 0.001 to about 50 wt % of hesperidin.

### Embodiment 31

The composition of one of Embodiments 27 to 30, comprising about 0.1 to about 10 wt % total ceramide.

### Embodiment 32

The composition of one of Embodiments 27 to 31, comprising about 0.01 to about 6 wt % total free fatty acid.

### Embodiment 33

The composition of one of Embodiments 27 to 32, comprising about 0.01 to about 5 wt % of the acid buffer.

### Embodiment 34

The composition of one of Embodiments 27 to 33, further comprising a skin conditioner.

### Embodiment 35

The composition of Embodiment 34, comprising about 0.1 to about 10 wt % of the skin conditioner.

### Embodiment 36

The composition of one of Embodiments 34 to 35, wherein the skin conditioner is dimethicone.

### Embodiment 37

The composition of one of Embodiments 27 to 36, further comprising a chelating agent.

### Embodiment 38

The composition of Embodiment 37, comprising about 0.001 to about 5 wt % of the chelating agent.

### Embodiment 39

The composition of one of Embodiments 37 to 38, wherein the chelating agent is disodium ethylenediaminetetraacetic acid (EDTA).

### Embodiment 40

The composition of one of Embodiments 27 to 39, further comprising a humectant.

### Embodiment 41

The composition of Embodiment 40, comprising about 1 to about 30 wt % of the humectant.

### Embodiment 42

The composition of one of Embodiments 40 to 41, wherein the humectant is glycerin.

### Embodiment 43

The composition of one of Embodiments 27 to 42, further comprising an emulsifier.

### Embodiment 44

The composition of Embodiment 43, comprising about 0.5 to about 20 wt % of the emulsifier.

### Embodiment 45

The composition of one of Embodiments 43 to 44, wherein the emulsifier is glyceryl stearate PEG-100.

### Embodiment 46

The composition of one of Embodiments 27 to 45, further comprising an emollient.

### Embodiment 47

The composition of Embodiment 46, comprising about 1 to about 30 wt % of the emollient.

### Embodiment 48

The composition of one of Embodiments 46 to 47, wherein the emollient is petrolatum.

### Embodiment 49

The composition of one of Embodiments 46 to 47, wherein the emollient is squalane.

### Embodiment 50

The composition of one of Embodiments 27 to 49, further comprising a preservative.

### Embodiment 51

The composition of Embodiment 50, comprising about 0.1 to about 10 wt % of the preservative.

### Embodiment 52

The composition of one of Embodiments 50 to 51, wherein the preservative is phenoxyethanol.

### Embodiment 53

The composition of one of Embodiments 50 to 51, wherein the preservative is sorbic acid.

### Embodiment 54

The composition of one of Embodiments 27 to 53, further comprising an encapsulation aid.

### Embodiment 55

The composition of Embodiment 54, comprising about 1 to about 25 wt % of the encapsulation aid.

### Embodiment 56

The composition of one of Embodiments 54 to 55, wherein the encapsulation aid is euphorbia cerifera (candelilla) wax.

### Embodiment 57

The composition of one of Embodiments 54 to 55, wherein the encapsulation aid is corn syrup solids.

### Embodiment 58

The composition of one of Embodiments 27 to 57, further comprising an aqueous phase thickener.

### Embodiment 59

The composition of Embodiment 58, comprising about 0.01 to about 3 wt % of the aqueous phase thickener.

### Embodiment 60

The composition of one of Embodiments 58 to 59, wherein the aqueous phase thickener is xanthan gum.

### Embodiment 61

The composition of one Embodiments 27 to 60, wherein the ceramide is hydroxypropyl bispalmitamide MEA (Ceramide PC-104).

### Embodiment 62

The composition of one of Embodiments 27 to 61, wherein the free fatty acid is conjugated linoleic acid (CLA).

### Embodiment 63

The compositions of one of Embodiments 27 to 62, further comprising a glucocorticoid.

### Embodiment 64

The composition of Embodiment 63, comprising about 0.001 to about 5 wt % of the glucocorticoid.

### Embodiment 65

The composition of one of Embodiments 27 to 64, wherein the composition is a topical composition.

### Embodiment 66

The composition of one of Embodiments 27 to 65, for use in treating dry skin in a subject in need thereof.

### Embodiment 67

The composition of one of Embodiments 27 to 65, for use in treating abnormal epidermal barrier function in a subject in need thereof.

### Embodiment 68

The composition of one of Embodiments 27 to 65, for use in treating an inflammatory condition in a subject in need thereof.

### Embodiment 69

The composition of one of Embodiments 27 to 65, for use in treating a clinical indication of skin aging in a subject in need thereof.

### Embodiment 70

The composition of Embodiment 69, wherein the clinical indication of skin aging is eczema, xerosis, xerotic eczema, winter itch, nummular eczema, seborrheic dermatitis, occupational dermatitis, hand eczema, or stasis dermatitis.

### Embodiment 71

The composition of one of Embodiments 27 to 65, for use in treating a dermal clinical indication of glucocorticoid treatment in a subject in need thereof.

### Embodiment 72

The composition of Embodiment 71, wherein the dermal clinical indication of glucocorticoid treatment is inflammation, tachyphylaxis, disease flares, or rebound flares.

### Embodiment 73

The composition of one of Embodiments 27 to 65, for use in treating a clinical indication of hypolipodemic treatment in a subject in need thereof.

### Embodiment 74

The method of Embodiment 20, wherein the aged skin is photo-aged skin.

1. A topical composition, said composition comprising:
a) about 1 to about 50 wt % of cholesterol;
b) about 0.01 to about 5 wt % of an acid buffer; and
c) about 0.001 to about 50 wt % of hesperidin;
wherein the pH of said topical composition is from about 3 to about 5.5.

2. A topical composition, said composition comprising:
a) a mixture of barrier lipids selected from the group consisting of cholesterol, at least one ceramide, at least one free fatty acid, and mixtures of two or more thereof, in a total of about 1 to about 50 wt %;
b) about 0.1 to about 10 wt % of a skin conditioner;
c) about 0.001 to about 5 wt % of a chelating agent;
d) about 1 to about 30 wt % of a humectant;
e) about 0.5 to about 20 wt % of an emulsifier;
f) one or more emollients in a total of about 1 to about 30 wt %;
g) one or more preservatives in a total of about 0.1 to about 10 wt %;
h) about 0.01 to about 5 wt % of an acid buffer;
i) one or more encapsulation aids in a total of about 1 to about 25 wt %;
j) about 0.01 to about 3 wt % of an aqueous phase thickener; and
k) about 0.001 to about 50 wt % of hesperidin.

3. The composition of claim 2, wherein said mixture of barrier lipids consists of cholesterol, hydroxypropyl bispalmitamide MEA (Ceramide PC-104) and conjugated linoleic acid (CLA); said skin conditioner comprises dimethicone; said chelating agent comprises disodium EDTA; said humectant comprises glycerin; and said acid buffer comprises citric acid; emulsifier comprises glyceryl stearate PEG-100; wherein said one or more emollients comprise petrolatum and squalane; wherein said one or more preservatives comprise phenoxyethanol and sorbic acid; wherein said one or more encapsulation aids comprise corn syrup solids and euphorbia cerifera (candelilla) wax; wherein said aqueous phase thickener comprises xanthan gum; wherein the pH of said topical composition is about 3 to about 5.5; and herein the molar ratio of cholesterol:ceramide:free fatty acid is 3:1:1.

4. The composition of claim 2, wherein cholesterol is present in about 0.1 to about 10 wt %; wherein said ceramide is present in about 0.1 to about 10 wt %; and wherein said free fatty acid is present in about 0.01 to about 6 wt %.

5. A composition comprising:
a) a mixture of barrier lipids selected from the group consisting of cholesterol, ceramides, free fatty acids and mixtures of two or more thereof, in a total of about 1 to about 50 wt %;
b) about 0.001 to about 50 wt % of hesperidin;
c) about 0.01 to about 5 wt % of an acid buffer; and
d) about 0.001 to about 5% of at least one glucocorticoid.

6. A method of treating aged skin, comprising administering the topical composition of claim 1 to at least a portion of aged skin, in an amount effective to relieve or reverse a clinical indication of skin aging, wherein said clinical indication of skin aging is selected from the group consisting of eczema, xerosis, xerotic eczema, winter itch, nummular eczema, seborrheic dermatitis, occupational dermatitis, hand eczema, and stasis dermatitis.

7. A method of preventing or reversing dermal side effects of systemic or topical glucocorticoid treatment, comprising treating at least a portion of affected skin with the topical composition of claim 1, in an amount effective to prevent or reverse dermal a clinical indication of glucocorticoid treatment, wherein said dermal clinical indication of glucocorticoid treatment is selected from the group consisting of inflammation, tachyphylaxis, disease flares, and rebound flares.

8. A method of treating dry skin or abnormal epidermal barrier function associated with oral hypolipodemic agent treatment, comprising administering the topical composition of claim 1 to at least a portion of affected skin of a subject, in an amount effective to relieve or reverse a clinical indication of hypolipodemic treatment.

9. A composition comprising cholesterol and hesperidin, wherein:
(i) said composition further comprises a ceramide and a free fatty acid wherein the mole ratio of total cholesterol:total ceramide:total free fatty acid is about 3:1:1 moles; or
(ii) the pH of the composition is from about 3 to about 5.5.

10. The composition of claim 9, wherein:
(i) said composition further comprises a ceramide and a free fatty acid wherein the mole ratio of total cholesterol:total ceramide:total free fatty acid is about 3:1:1 moles; and
(ii) the pH of the composition is from about 3 to about 5.5.

11. The composition of claim 9, comprising about 1 to about 50 wt % of total cholesterol; comprising about 0.1 to about 10 wt % total ceramide; comprising about 0.01 to about 6 wt % total free fatty acid; and comprising about 0.001 to about 50 wt % of hesperidin.

12. The composition of claim 9, further comprising an acid buffer.

13. The composition of claim 9, further comprising a skin conditioner; a humectant; an emulsifier; a chelating agent; and an emollient.

14. The composition of claim 9, further comprising a preservative; an aqueous phase thickener; and an encapsulation aid.

15. The composition of claim 9, further comprising a glucocorticoid.

16. A method for treating dry skin said method comprising administering to a subject in need thereof an effective amount of composition of claim 9.

17. A method for treating abnormal epidermal barrier function said method comprising administering to a subject in need thereof an effective amount of composition of claim 9.

18. A method for treating an inflammatory condition said method comprising administering to a subject in need thereof an effective amount of composition of claim 9.

19. A method for treating a clinical indication of skin aging said method comprising administering to a subject in need thereof an effective amount of composition of claim 9, wherein said clinical indication of skin aging is selected from the group consisting of eczema, xerosis, xerotic eczema, winter itch, nummular eczema, seborrheic dermatitis, occupational dermatitis, hand eczema, and stasis dermatitis.

20. A method for treating a dermal clinical indication of glucocorticoid treatment said method comprising administering to a subject in need thereof an effective amount of composition of claim 9, wherein said dermal clinical indication of glucocorticoid treatment is selected from the group consisting of inflammation, tachyphylaxis, disease flares, and rebound flares.
